(19) United States
(12) Patent Application Publication
(10) Pub. No.: US 2019/0185568 A1
(43) Pub. Date: Jun. 20, 2019

(54) ANTI-PD-1 ANTIBODY

(71) Applicants: Fuso Pharmaceutical Industries, Ltd., Osaka (JP); National University Corporation Hokkaido University, Sapporoshi, Hokkaido (JP)

(72) Inventors: Satoru Konnai, Sapporoshi, Hokkaido (JP); Kazuhiko Ohashi, Sapporoshi, Hokkaido (JP); Shiro Murata, Sapporoshi, Hokkaido (JP); Tomohiro Okagawa, Sapporoshi, Hokkaido (JP); Asami Nishimori, Sapporoshi, Hokkaido (JP); Naoya Maekawa, Sapporoshi, Hokkaido (JP); Yasuhiko Suzuki, Sapporoshi, Hokkaido (JP); Chie Nakajima, Sapporoshi, Hokkaido (JP)

(21) Appl. No.: 16/325,144

(22) PCT Filed: Aug. 10, 2017
(86) PCT No.: PCT/JP2017/029056

§ 371 Date: Feb. 12, 2019

(30) Foreign Application Priority Data
Aug. 15, 2016 (JP) 2016-159090
May 19, 2017 (JP) 2017-099615

(51) Int. Cl. C07K 16/28; A61P 35/02; C07K 16/46; C12N 15/85; C12N 5/12; A61P 33/02; A61P 31/04

(52) U.S. Cl. CPC C07K 16/2818; A61P 35/02; C07K 16/46; A61K 38/00; C12N 5/12; A61P 33/02; A61P 31/04; C12N 15/85

(57) ABSTRACT

The present invention provides an anti-PD-1 antibody capable of repeated administration even to animals other than rat. An anti-PD-1 antibody comprising (a) a light chain comprising a light chain variable region containing CDR1 having the amino acid sequence of QSLEYSDGYTY (SEQ ID NO: 16), CDR2 having the amino acid sequence of GVS and CDR3 having the amino acid sequence of FQATHDPDT (SEQ ID NO: 17) and the light chain constant region of an antibody of an animal other than rat; and (b) a heavy chain comprising a heavy chain variable region containing CDR1 having the amino acid sequence of GFSLTSYY (SEQ ID NO: 18), CDR2 having the amino acid sequence of IRSGGST (SEQ ID NO: 19) and CDR3 having the amino acid sequence of ARTSSGYEGGFDY (SEQ ID NO: 20) and the heavy chain constant region of an antibody of an animal other than rat. A pharmaceutical composition comprising the above-described anti-PD-1 antibody as an active ingredient. A method for preparing the anti-PD-1 antibody is also provided.

### TECHNICAL FIELD

The present invention relates to an anti-PD-1 antibody. More specifically, the present invention relates to an anti-PD-1 antibody comprising a variable region containing complementarily-determining regions (CDRs) of a rat anti-bovine PD-1 antibody and a constant region of an antibody of an animal other than rat.

### BACKGROUND ART

Programmed cell death 1 (PD-1), an immunoinhibitory receptor, and its ligand programmed cell death ligand 1 (PD-L1) are molecules identified by Prof. Tasuku Honjo et al., Kyoto University, as factors which inhibit excessive immune response and are deeply involved in immunotolerance (Non-Patent Document No. 1: Ishida Y, Agata Y, Shibahara K, Honjo T. The EMBO J., 11(11); Nov. 1992). Recently, it has been elucidated that these molecules are also involved in immunosuppression in tumors. In the field of human medical care, an antibody drug that inhibits the effect of PD-1 has been developed and put into practical use (Opdivo™, Ono Pharmaceutical Co., Ltd.).

To date, the present inventors have been developing an immunotherapy for animal refractory diseases targeting PD-1 or PD-L 1, and have revealed that this novel immunotherapy is applicable to multiple-diseases and multiple-animals. (Non-Patent Document No. 2: Ikebuchi R, Konnai S, Shirai T, Sunden Y, Murata S, Onuma M, Ohashi K. Vet. Res.; 42: 103; Sep. 2011; Non-Patent Document No. 3: Ikebuchi R, Konnai S; Okagawa T, Yokoyama K, Nakajima C, Suzuki Y, Murata S, Ohashi K. Vet. Res., 44: 59; Jul. 22, 2013; Non-Patent Document No. 4: Ikebuchi R, Konnai S, Okagawa T Yokoyama K, Nakajima C, Suzuki Y, Murata S, Ohashi K. Immunology, 142(4): 551-61; Aug. 2014; Non-Patent Document No. 5: Maekawa N, Konnai S, Ikebuchi R, Okagawa T, Adachi M, Takagi S, Kagawa Y, Nakajima C, Suzuki Y, Murata S, Ohashi K. PLoS One, 9(6): e98415; Jun. 10, 2014; Non-Patent Document No. 6: Mingala C N, Konnai S, Ikebuchi R, Ohashi K. Comp. Immunol. Microbiol. Infect. Dis., 34(1): 55-63; Jan. 2011).

However, the antibodies which the present inventors have prepared to date are rat antibodies, and therefore it is impossible to administer those antibodies repeatedly to animals other than rat.

### PRIOR ART LITERATURE

### Non-Patent Documents

### Non-Patent Document No. 1: Ishida Y, Agata Y, Shibahara K, Honjo T. The EMBO Journal. 11(11):3887-3895; Nov. 1992

Non-Patent Document No. 2: Ikebuchi R, Konnai S, Shirai T, Sunden Y Murata S, Onuma M, Ohashi K. Vet. Res., 42: 103; Sep. 2011.
Non-Patent Document No. 3: Ikebuchi R, Konnai S, Okagawa T, Yokoyama K, Nakajima C, Suzuki Y, Murata S, Ohashi K. Vet. Res., 44: 59; Jul. 22, 2013.

### Non-Patent Document No. 4: Ikehuchi R, Konnai S, Okagawa T, Yokoyama K, Nakajima C, Suzuki Y Murata S, Ohashi K. Immunology, 142(4): 551-61; Aug. 2014.

Non-Patent Document No. 5: Maekawa N, Konnai S, Ikebuchi R, Okagawa T, Adachi M, Takagi S, Kagawa Y, Nakajima C, Suzuki Y, Murata S, Ohashi K. PLoS One, 9(6): e98415; Jun. 10, 2014.
Non-Patent Document No. 6: Mingala C N, Konnai S. Ikebuchi R, Ohashi K. Comp. Immunol. Microbiol. Infect. Dis., 34(1): 55-63; Jan. 2011.

### DISCLOSURE OF THE INVENTION

### Problem for Solution by the Invention

It is an object of the present invention to provide an anti-PD-1 antibody capable of repeated administration even to animals other than rat.

### Means to Solve the Problem

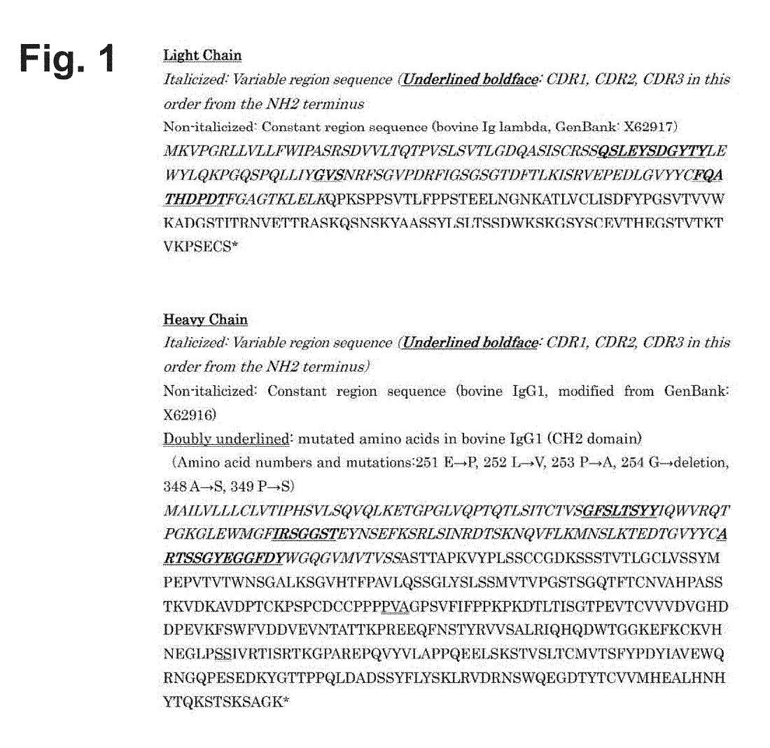

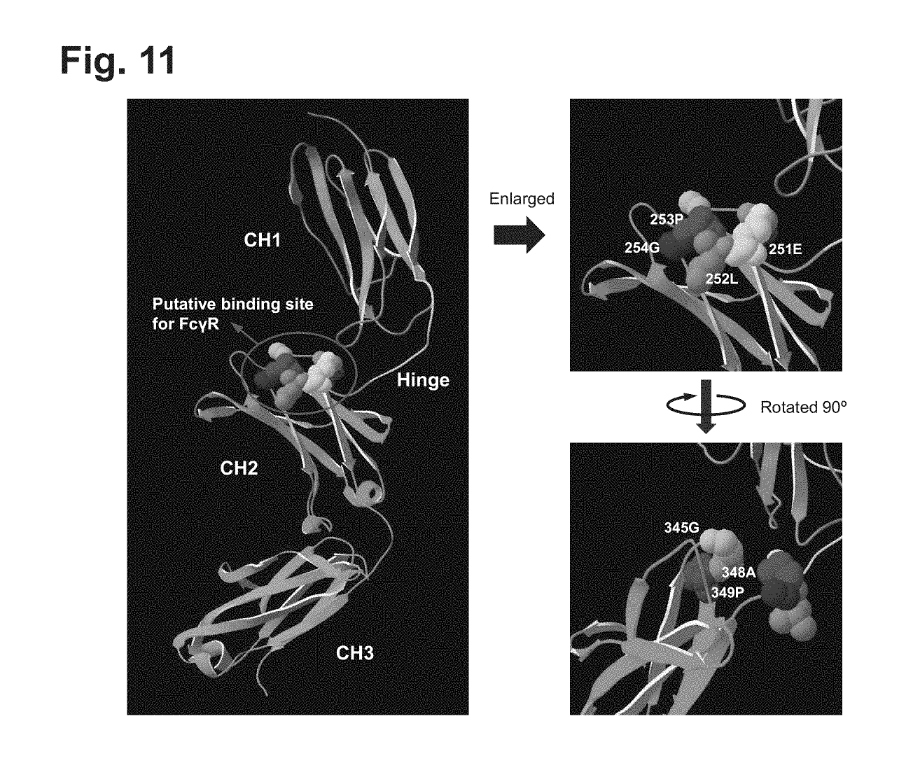

The present inventors have determined the variable regions of a rat anti-bovine PD-1 monoclonal antibody (5D2) capable of inhibiting the binding of bovine PD-1 to PD-L1, and then combined genes encoding the resultant variable regions with genes encoding the constant regions of a bovine immunoglobulin (bovine IgG1, with mutations having been introduced into the putative binding sites of Fcγ receptors in CH2 domain in order to inhibit ADCC activity; see FIGS. 1 and 11 for amino acid numbers and mutations: 251 E→P, 252 L→V, 253 P→A, 254 G→deletion, 348 A→S, 349 P→S; Ikebuchi R, Konnai S, Okagawa T, Yokoyama K, Nakajima C, Suzuki Y, Murata S, Ohashi K. Immunology 2014 Aug; 142(4): 551-561) to thereby obtain a chimeric antibody gene. This gene was introduced into Chinese hamster ovary cells (CHO cells). By culturing/proliferating the resultant cells, the present inventors have succeeded in preparing a rat-bovine chimeric anti-bovine PD-1 antibody Further, the present inventors have determined the CDRs of the variable regions of rat anti-bovine PD-1 monoclonal antibody (5D2). The present invention has been achieved based on these findings.

A summary of the present invention is as described below.

(1) An anti-PD-1 antibody comprising (a) a light chain comprising a light chain variable region containing CDR1 having the amino acid sequence of QSLEYSDGYTY (SEQ ID NO: 16), CDR2 having the amino acid sequence of GVS and CDR3 having the amino acid sequence of FQATHDPDT (SEQ ID NO: 17) and the light chain constant region of an antibody of an animal other than rat; and (b) a heavy chain comprising a heavy chain variable region containing CDR1 having the amino acid sequence of GFSLTSYY (SEQ ID NO: 18), CDR2 having the amino acid sequence of IRSGGST (SEQ ID NO: 19) and CDR3 having the amino acid sequence of ARTSSGVEGGFDY (SEQ ID NO: 20) and the heavy chain constant region of an antibody of an animal other than rat.
(2) The antibody of (1) above, wherein the light chain variable region and the heavy chain variable region are derived from rat.
(3) The antibody of (2) above, wherein the light chain variable region is the light chain variable region of a rat anti-bovine PD-1 antibody and the heavy chain variable region is the heavy chain variable region of a rat anti-bovine PD-1 antibody.
(4) The antibody of (3) above, wherein the light chain variable region has the amino acid sequence as shown in SEQ ID NO. 1 and the heavy chain variable region has the amino acid sequence as shown in SEQ ID NO: 2.
(5) The antibody of any one of (1) to (4) above, wherein the light chain constant region of an antibody of an animal other than rat has the amino acid sequence of the constant region of lambda chain or kappa chain.
(6) The antibody of any one of (1) to (5) above, wherein the heavy chain constant region of an antibody of an animal other than rat has the amino acid sequence of the constant region of an immunoglobulin equivalent to human IgG4, or has mutations introduced thereinto that reduce ADCC activity and/or CDC activity.
(7) The antibody of (6) above, wherein the animal other than rat is bovine; the light chain constant region of the bovine antibody has the amino acid sequence of the constant region of lambda chain; and the heavy chain constant region of the bovine antibody has mutations introduced thereinto that reduce ADCC activity and/or CDC activity.
(8) The antibody of (7) above, wherein the light chain constant region of the bovine antibody has the amino acid sequence as shown in SEQ ID NO: 3 and the heavy chain constant region of the bovine antibody has the amino acid sequence as shown in SEQ ID NO: 4
(9) The antibody of any one of (1) to (8) above which has a four-chain structure comprising two light chains and two heavy chains.
(10) A pharmaceutical composition comprising the antibody of any one of (1) to (9) above as an active ingredient.
(11) The composition of (10) above for prevention and/or treatment of cancers and/or inflammations.
(12) The composition of (11) above, wherein the cancers and/or inflammations are selected from the group consisting of neoplastic diseases, leukemia, Johne's disease, anaplasmosis, bacterial mastitis, mycotic mastitis, mycoplasma infections (such as mycoplasma mastitis, mycoplasma pneumonia or the like), tuberculosis, Theileria orientaiis infection, cryptosporidiosis, coccidiosis, trypanosomiasis and leishmaniasis.
(13) An artificial genetic DNA comprising (a′) a DNA encoding a light chain comprising a light chain variable region containing CDR1 having the amino acid sequence of QSLEYSDGYTY (SEQ ID NO: 16), CDR2 having the amino acid sequence of GVS and CDR3 having the amino acid sequence of FQATHDPDT (SEQ ID NC): 17) and the light chain constant region of an antibody of an animal other than rat and (b′) a DNA encoding a heavy chain comprising a heavy Chain variable region containing CDR1 having the amino acid sequence of GFSLTSYY (SEQ ID NO: 18), CDR2 having the amino acid sequence of IRSGGST (SEQ ID NO: 19) and CDR3 having the amino acid sequence of ARTSSGYEGGFDY (SEQ ID NO: 20) and the heavy chain constant region of an antibody of an animal other than rat.
(14) A vector comprising the artificial genetic DNA of (13) above.
(15) A host cell transformed with the vector of (14) above.
(16) A method of preparing an antibody, comprising culturing the host cell of (15) above and collecting an anti-PD-1 antibody from the resultant culture.
(17) A DNA encoding a light chain comprising a light chain variable region containing CDR1 having the amino acid sequence of QSLEYSDGYTY (SEQ ID NO: 16), CDR2 having the amino acid sequence of GVS and CDR3 having the amino acid sequence of FQATHDPDT (SEQ ID NO: 17) and the light chain constant region of an antibody of an animal other than rat.
(18) A DNA encoding a heavy chain comprising a heavy chain variable region containing CDR1 having the amino acid sequence of GFSLTSYY (SEQ ID NO: 18), CDR2 having the amino acid sequence of IRSGGST (SEQ ID NO: 19) and CDR3 having the amino acid sequence of ARTSSGYEGGFDY (SEQ ID NO: 20) and the heavy chain constant region of an antibody of an animal other than rat.

### Effect of the Invention

According to the present invention, a novel anti-PD-1 antibody has been obtained. This antibody is applicable even to those animals other than rat.

The present specification encompasses the contents disclosed in the specifications and/or drawings of Japanese Patent Applications No. 2016-159090 and No. 2017-099615 based on which the present patent application claims priority.

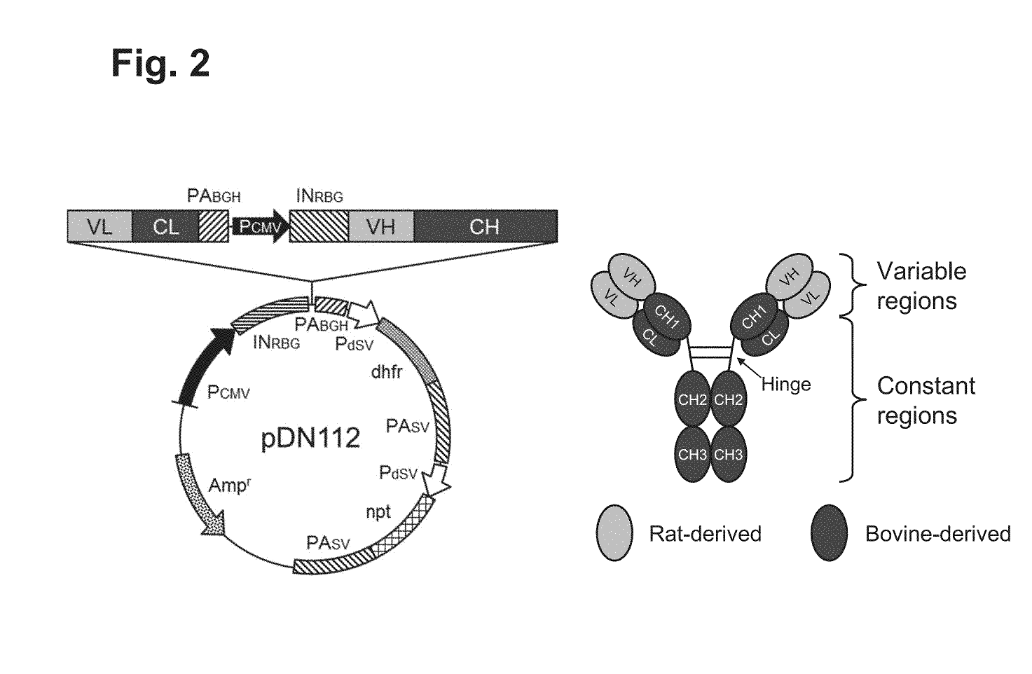

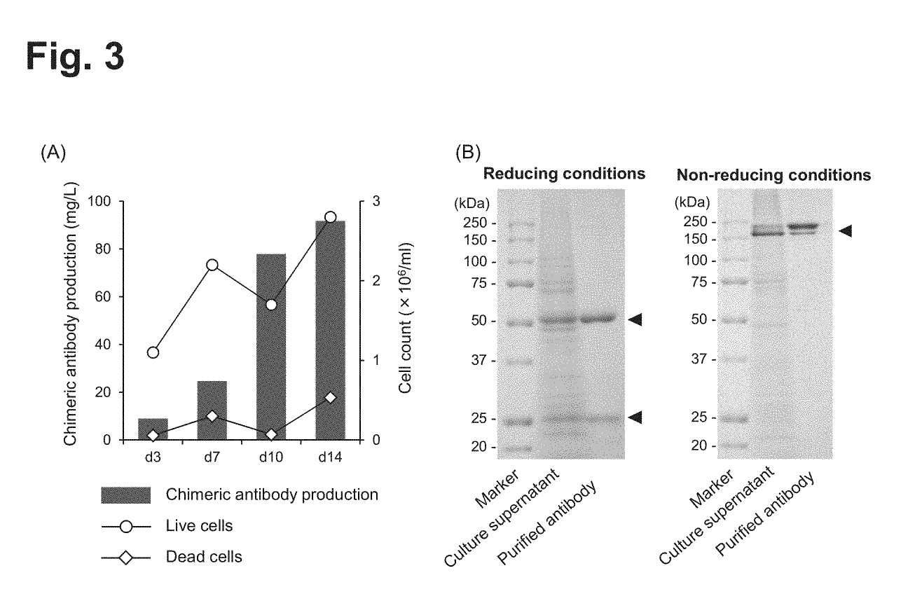

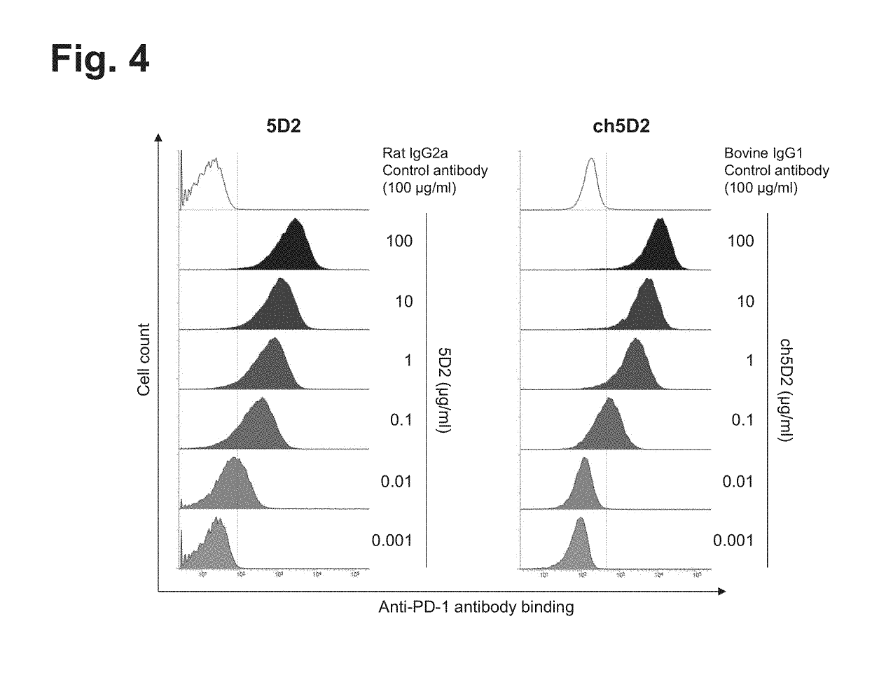

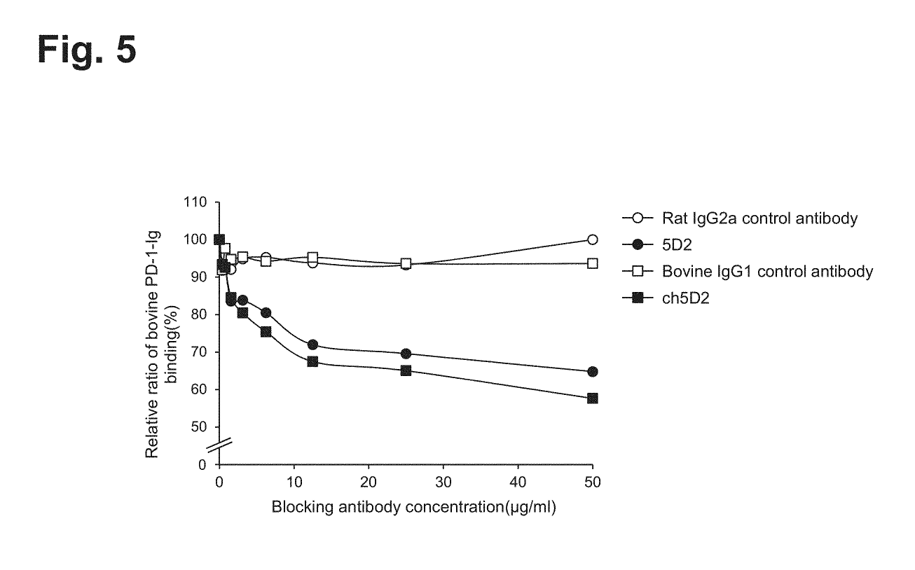

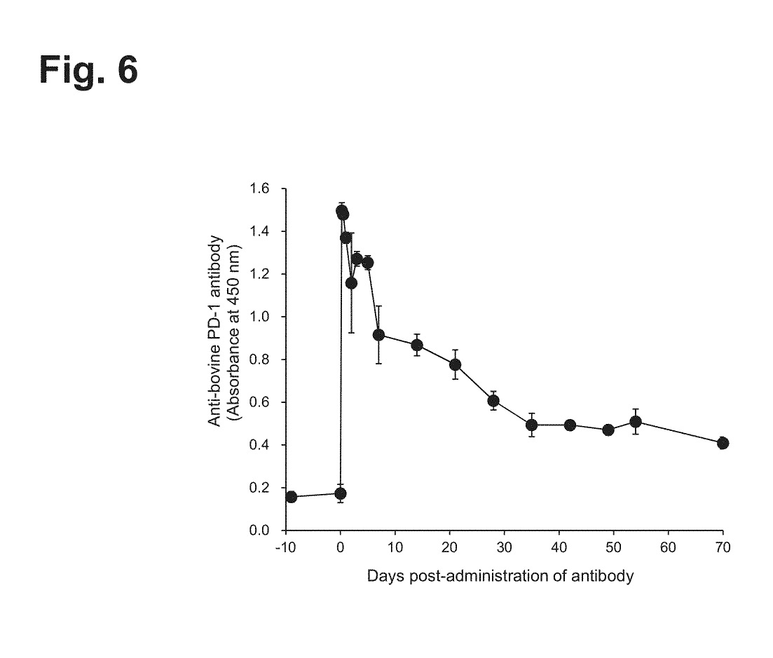

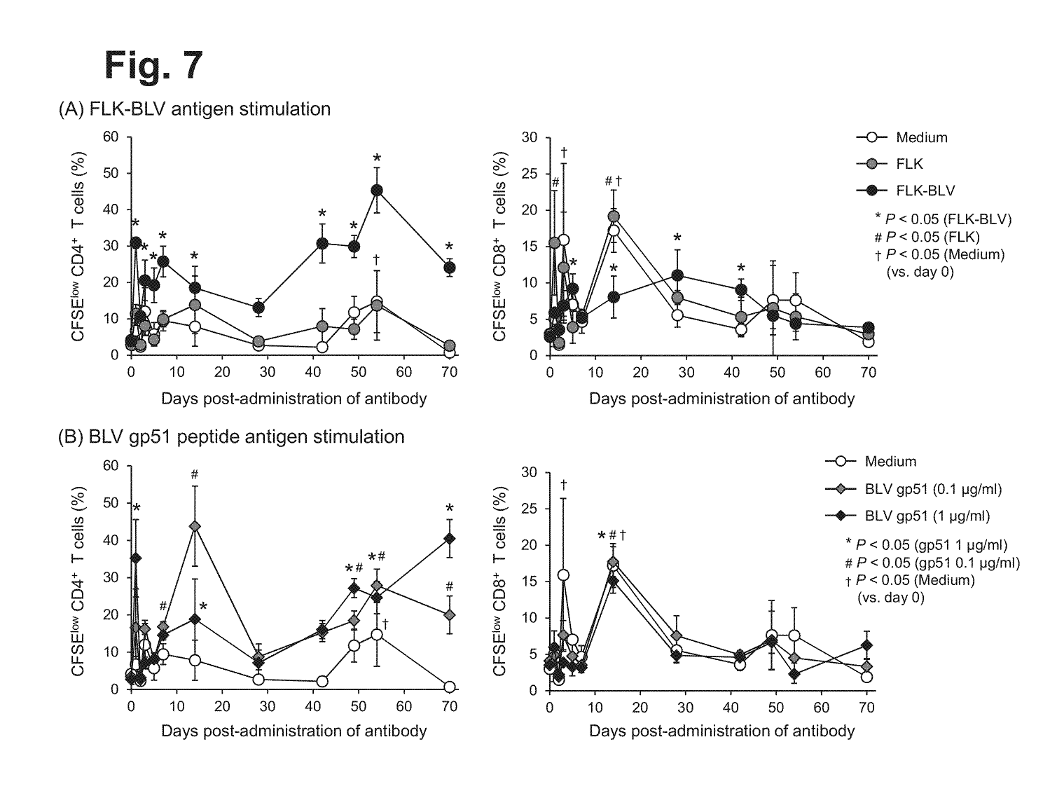

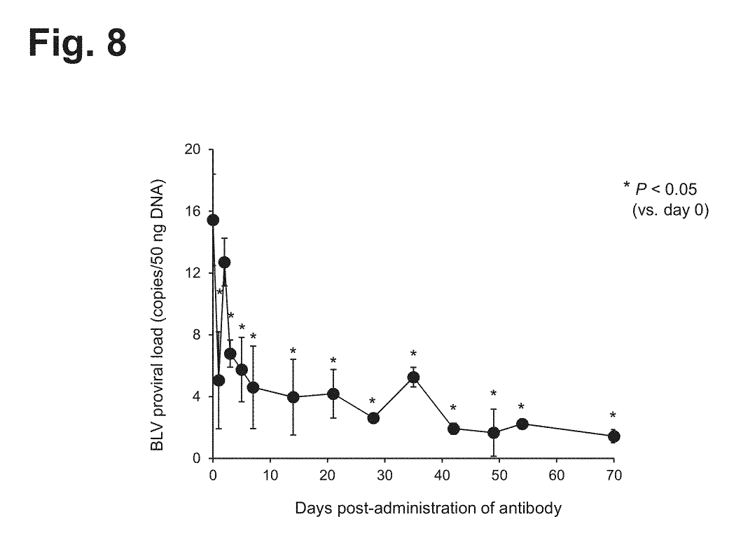

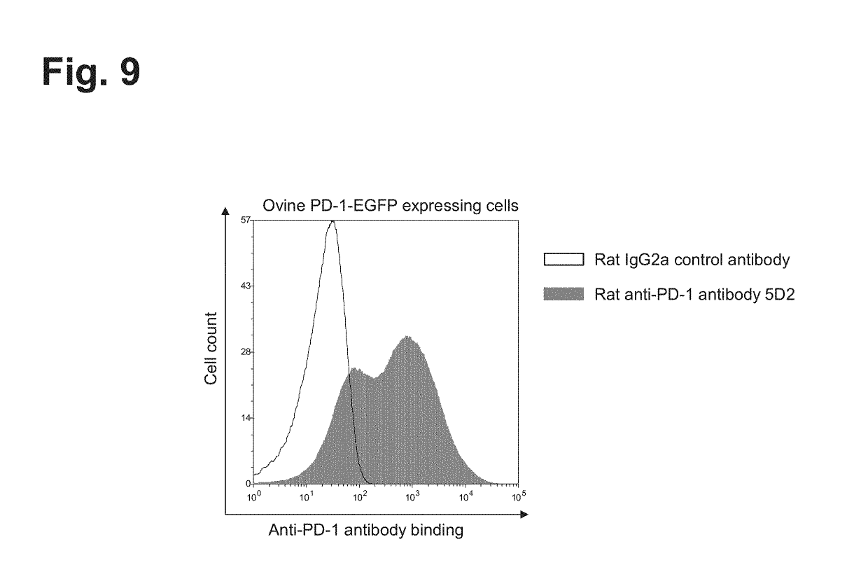

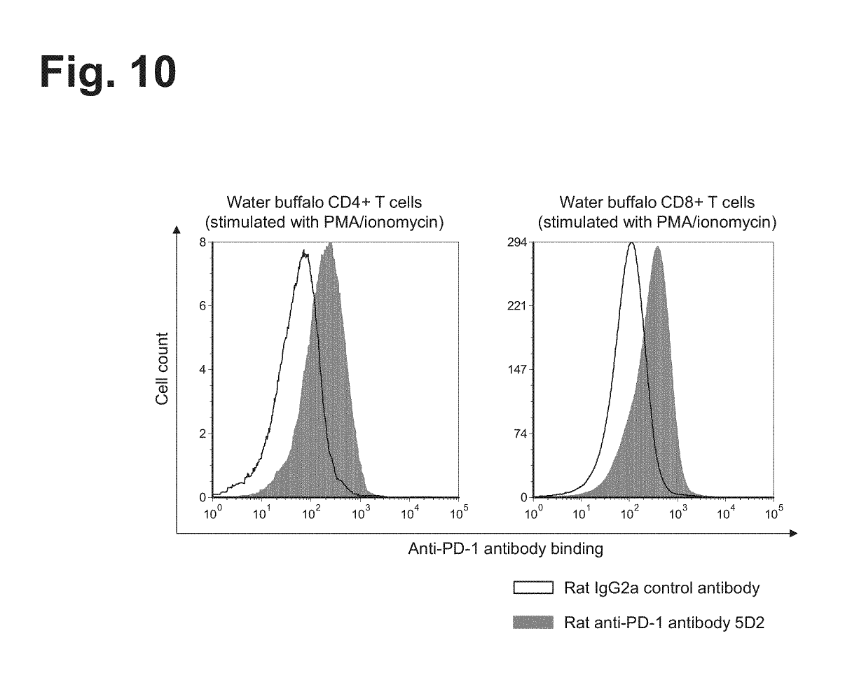

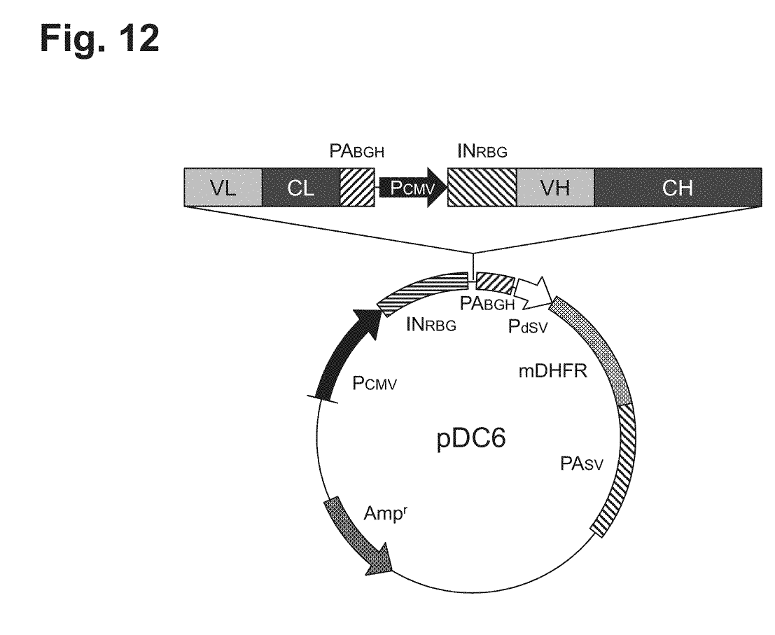

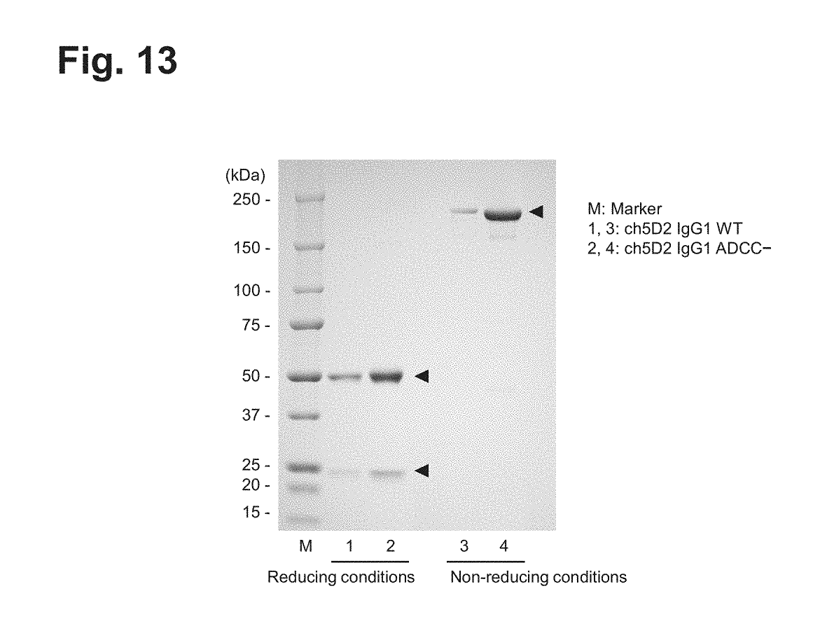

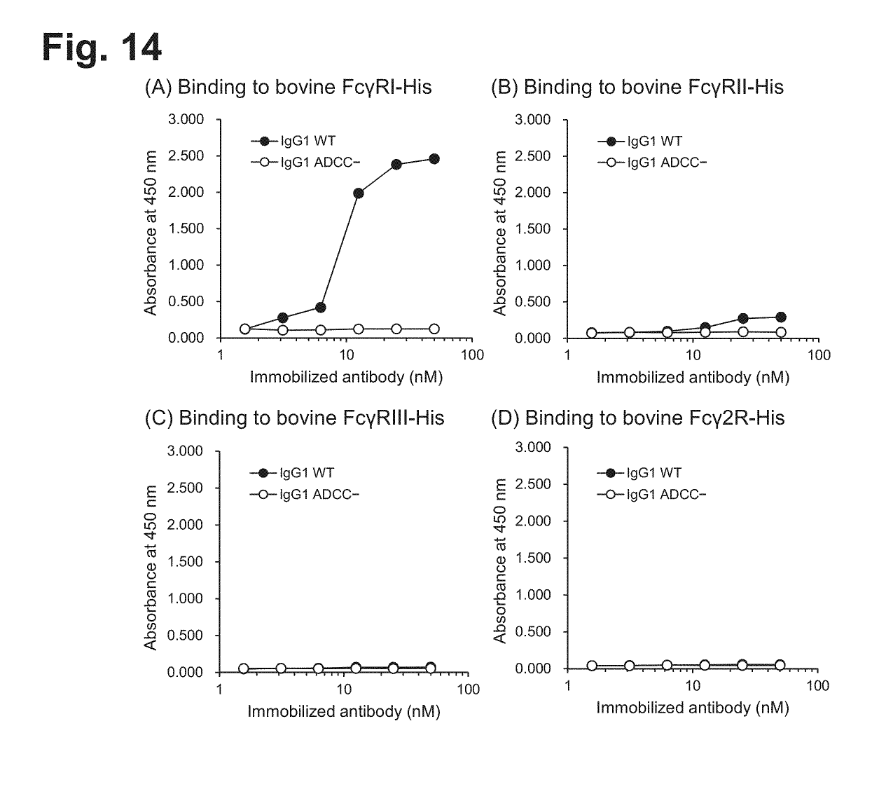

### BRIEF DESCRIPTION OF THE DRAWINGS

[FIG. 1] The amino acid sequence of rat-bovine chimeric anti-bovine PD-1 antibody ch5D2. CDR1, CDR2 and CDR3 in the light chain and the heavy chain variable regions of rat anti-bovine PD-1 antibody 5D2 are shown. Further, amino acids introduced as mutations to bovine IgG4 (CH2 domain) are also shown (amino acid numbers and mutations: 251 E→P, 252 L→V, 253 P→A, 254 G→deletion, 348 A→S, 349 P→S).

[FIG. 2] Schematic drawings of pDN112 vector and rat-bovine chimeric anti-bovine PD-1 antibody ch5D2.

[FIG. 3] The amount of production and the purity after purification of rat-bovine chimeric anti-bovine PD-1 antibody ch5D2.

[FIG. 4] Binding property of rat-bovine chimeric anti-bovine PD-1 antibody ch5D2.

[FIG. 5] Inhibitory activity of rat-bovine chimeric anti-bovine PD-1 antibody ch5D2 against bovine PD-1/PD-L1 binding.

[FIG. 6] Transition in blood concentrations of rat-bovine chimeric anti-bovine PD-1 antibody ch5D2 after administration to the cattle experimentally infected with BLV.

[FIG. 7] Proliferative response of T cells against BLV antigen in the cattle experimentally infected with BLV through administration of rat-bovine chimeric anti-bovine PD-1 antibody ch5D2.

[FIG. 8] Changes in the BLV provirus load in the cattle experimentally infected with BLV through administration of rat-bovine chimeric anti-bovine PD-1 antibody ch5D2.

[FIG. 9] Cross-reactivity of rat anti-bovine PD-1 antibody 5D2 with ovine PD-1

[FIG. 10] Cross-reactivity of rat anti-bovine PD-1 antibody 5D2 with water buffalo T cells

[FIG. 11] 3D structure of bovine IgG1 constant region and putative binding site for Fcγ receptors

[FIG. 12] pDC6 vector

[FIG. 13] Purities after purification of rat-bovine chimeric anti-bovine PD-1 antibodies ch5D2 IgG1 WT and IgG1 ADCC-.

[FIG. 14] Binding of rat-bovine chimeric anti-bovine PD-1 antibodies ch5D2 IgG1 WT and IgG1. ADCC- to individual bovine Fcγ receptors.

### BEST MODES FOR CARRYING OUT THE INVENTION

Hereinbelow, the present invention will be described in detail.

The present invention provides an anti-PD-1 antibody comprising light chain comprising a light chain variable region containing CDR1 having the amino acid sequence of QSLEYSDGYTY (SEQ ID NO: 16), CDR2 having the amino acid sequence of GVS and CDR3 having the amino acid sequence of FQATHDPDT (SEQ ID NO: 17) and the light chain constant region of an antibody of an animal other than rat; and (b) a heavy chain comprising a heavy chain variable region containing CDR1 having the amino acid sequence of GFSLTSYY (SEQ ID NO: 18), CDR2 having the amino acid sequence of IRSGGST (SEQ ID NO: 19) and CDR3 having the amino acid sequence of ARTSSGYEGGFDY (SEQ ID NO: 20) and the heavy chain constant region of an antibody of an animal other than rat.

CDR1, CDR2 and CDR3 in the light chain variable region (VL) of rat anti-bovine PD-1 antibody 5D2 (to be described later) are respectively a region consisting of the amino acid sequence of QSLEYSDGYTY (SEQ ID NO: 16), a region consisting of the amino acid sequence of GVS and a region consisting of the amino acid sequence of FQATHDPDT (SEQ ID NO: 17) (see FIG. 1).

Further, CDR1, CDR2 and CDR3 in the heavy chain variable region (VH) of rat anti-bovine PD-1 antibody 5D2 are respectively a region consisting of the amino acid sequence of GFSLTSYY (SEQ ID NO: 18), a region consisting of the amino acid sequence of IRSGGST (SEQ ID NO: 19) and a region consisting of the amino acid sequence of ARTSSGYEGGFDY (SEQ ID NO: 20) (see FIG. 1).

In the amino acid sequences of QSLEYSDGYTY (SEQ ID NO: 16), GVS and FQATHDPDT (SEQ ID NO: 17), as well as the amino acid sequences of GFSLTSYY (SEQ ID NO: 18), IRSGGST (SEQ ID NO: 19) and ARTSSGYEGGFDY (SEQ ID NO: 20), one, two, three, four or five amino acids may be deleted, substituted or added. Even when such mutations are introduced, the resulting amino acid sequences may be capable of having the function of a CDR in the light chain or heavy chain variable region of the PD-1 antibody.

As used herein, the term “antibody” is a concept encompassing not only full-length antibodies but also antibodies of smaller molecular sizes such as Fab, F(ab)′₂, ScFv, Diabody, V_{H}, V_{L}, Sc(Fv)₂, Bispecific sc(Fv)₂, Minibody, scFv-Fc monomer or scFv-Fc dimer.

In the anti-PD-1 antibody of the present invention, the VL and VH may be derived from rat. For example, the VL may be the VL of a rat anti-bovine PD-1 antibody, and the VH may be the VH of the rat anti-bovine PD-1 antibody.

The amino acid sequence of the VL and the amino acid sequence of the VH of the rat anti-bovine PD-1 antibody are shown in SEQ ID NOS: 1 and 2, respectively. The amino acid sequences as shown in SEQ ID NOS: 1 and 2 may have deletion(s), substitution(s) or addition(s) of one or several (e.g., up to five, about 10 at the most) amino acids. Even when such mutations are introduced, the resulting amino acid sequences may be capable of having the function as VL or VH of the PD-1 antibody.

The VL and VH of an antibody of an animal other than rat may be derived from an animal which produces a PD-1 that cross-reacts with rat anti-bovine PD-1 antibody 5D2.

There are two types of immunoglobulin light chain, which are called Kappa chain (κ) and Lambda chain (λ). In the anti-PD-1 antibody of the present invention, the light chain constant region (CL) of an antibody of an animal other than rat may have the amino acid sequence of the constant region of either Kappa chain or Lambda chain. However, the relative abundance of Lambda chain is higher in bovine, ovine, feline, canine and equine, and that of Kappa chain is higher in mouse, rat, human and porcine. Since a chain with a higher relative abundance is considered to be preferable, a bovine, ovine, feline, canine or equine antibody preferably has the amino acid sequence of the constant region of Lambda chain whereas a mouse, rat, human or porcine antibody preferably has the amino acid sequence of the constant region of Kappa chain.

The heavy chain constant region (CH) of an antibody of an animal other than rat may have the amino acid sequence of the constant region of an immunoglobulin equivalent to human IgG4. Immunoglobulin heavy chain is classified into γ chain, μ chain, α chain, δ chain and ϵ chain depending on the difference in constant region. According to the type of heavy chain present, five classes (isotypes) of immunoglobulin are format they are IgG, IgM, IgA, IgD and IgE.

Immunoglobulin G (IgG) accounts for 70-75% of human immunoglobulins and is the most abundantly found monomeric antibody in plasma. IgG has a four-chain structure consisting of two light chains and two heavy chains. Human IgG1, IgG-2 and IgG4 have a molecular weight of about 146,000, whereas human IgG3 has a long hinge region that connects Fab region and Fc region and has a larger molecular weight of 170,000. Human IgG1 accounts for about 65% of human IgG, human IgG2 about 25%, human IgG3 about 7%, and human IgG4 about 3%. They are uniformly distributed inside and outside of blood vessels. Having a strong affinity for Fc receptors and complement factors on effector cell surfaces, human IgG1 induces antibody-dependent cell cytotoxicity (ADCC) and also activates complements to induce complement-dependent cell cytotoxicity (CDC). Human IgG2 and IgG4 are low at ADCC and CDC activities because their affinity for Fc receptors and complement factors is low.

Immunoglobulin M (IgM), which accounts for about 10% of human immunoglobulins, is a pentameric antibody consisting of five basic four-chain structures joined together. It has a molecular weight of 970,000. Usually occurring only in blood, IgM is first produced against infectious microorganisms and takes charge of early stage immunity.

Immunoglobulin A (IgA) accounts for 10-15% of human immunoglobulins. It has a molecular weight of 160,000. Secreted IgA is a dimeric antibody consisting of two IgA molecules joined together. IgA1 is found in serum, nasal discharge, saliva and breast milk. In intestinal juice, IgA2 is found abundantly.

Immunoglobulin D (IgD) is a monomeric antibody accounting for no more than 1% of human immunoglobulins. IgD is found on B cell surfaces and involved in induction of antibody production.

Immunoglobulin E (IgE) is a monomeric antibody that occurs in an extremely small amount, accounting for only 0.001% or less of human immunoglobulins. Immunoglobulin E is considered to be involved in immune response to parasites but in advanced countries where parasites are rare, IgE is largely involved in bronchial asthma and allergy among other things.

In canine, sequences of IgG-A (equivalent to human IgG2), IgG-B (equivalent to human IgG1), IgG-C (equivalent to human IgG3) and IgG-D (equivalent to human IgG4) have been identified as the heavy chain of IgG. In the antibody of the present invention, an IgG's heavy chain constant region with neither ADCC activity nor CDC activity is preferable (IgG4 in human). In the case where the constant region of an immunoglobulin equivalent to human IgG4 has not been identified, one may use a constant region that has lost both ADCC activity and CDC activity as a result of introducing mutations into the relevant region of an immunoglobulin equivalent to human IgG4.

In bovine, sequences of IgG1, IgG2 and IgG3 have been identified as the heavy chain of IgG. In the antibody of the present invention, an IgG's heavy chain constant region with neither ADCC activity nor CDC activity is preferable (IgG4 in human). Although the constant region of wild-type human IgG1 has ADCC activity and CDC activity, it is known that these activities can be reduced by introducing amino acid substitutions or deletions into specific sites. In bovine, the constant region of an immunoglobulin equivalent to human IgG4 has not been identified, so mutations may be added to the relevant region of an immunoglobulin equivalent to human IgG1 and the resultant constant region then used. As one example, the amino acid sequence of the CH of a bovine antibody (IgG1 chain, GenBank: X62916) having mutations introduced into CH2 domain and a nucleotide sequence for such amino acid sequence (after codon optimization) are shown in SEQ ID NOS: 4 and 8, respectively.

An anti-PD-1 antibody is more preferable in which (i) the CL of a bovine antibody has the amino acid sequence of the constant region of Lambda chain and (ii) the CH of the bovine antibody has mutations introduced thereinto that reduce ADCC activity and/or CDC activity.

The anti-PD-1 antibody of the present invention encompasses rat-bovine chimeric antibodies, bovinized antibodies and complete bovine-type antibodies. However, the animal is not limited to bovine and may be exemplified by human, canine, porcine, simian, mouse, feline, equine, goat, ovine, water buffalo, rabbit, hamster, guinea pig and the like.

For example, the anti-PD-1 antibody of the present invention may be an anti-PD-1 antibody in which the CL of a bovine antibody has the amino acid sequence as shown in SEQ ID NO: 3 and the CH of the bovine antibody has the amino acid sequence as shown in SEQ ID NO: 4.

The amino acid sequences as shown in SEQ ID NOS: 3 and 4 may have deletion(s), substitution(s) or addition(s) of one or several (e.g., up to five, about 10 at the most) amino acids. Even when such mutations are introduced, the resulting amino acid sequences may be capable of having the function as CL or CH of the PD-1 antibody.

The anti-PD-1 antibody of the present invention may have a four-chain structure comprising two light chains and two heavy chains.

The anti-PD-1 antibody of the present invention may be prepared as described below. Briefly, an artificial gene is synthesized which comprises (i) the identified variable region sequences of a rat anti-bovine PD-1 antibody and (ii) the constant region sequences of an antibody of an animal other than rat (e.g., bovine) (preferably, an immunoglobulin equivalent to human IgG1, in which mutations have been introduced into the relevant region to reduce ADCC activity and/or CDC activity). The resultant gene is inserted into a vector (e.g., plasmid), which is then introduced into a host cell (e.g., mammal cell such as CHO cell), The host cell is cultured, and the antibody of interest is collected from the resultant culture.

The amino acid sequence and the nucleotide sequence of the VL of the rat anti-bovine PD-1 antibody identified by the present inventors are shown in SEQ ID NOS: 1 and 5, respectively. Further, the nucleotide sequence after codon optimization is shown in SEQ ID NO: 11.

The amino acid sequence and the nucleotide sequence of the VH of the rat anti-bovine PD-1 antibody identified by the present inventors are shown in SEQ ID NOS: 2 and 6, respectively. Further, the nucleotide sequence after codon optimization is shown in SEQ ID NO: 12.

The amino acid sequence and the nucleotide sequence of the CL (Lambda chain, GenBank: X62917) of a bovine antibody are shown in SEQ ID NOS: 3 and 7, respectively. Further, the nucleotide sequence after codon optimization is shown in SEQ ID NO: 13.

The amino acid sequence and the nucleotide sequence (after codon optimization) of the CH (IgG1 chain, modified from GenBank: X62916) of a bovine antibody are shown in SEQ ID NOS: 4 and 8, respectively.

Further, SEQ ID NO: 9 shows the amino acid sequence of a chimeric light chain consisting of the VL of the rat anti-bovine PD-1 antibody and the CL (Lambda chain, GenBank: X62917) of the bovine antibody. The nucleotide sequence (after codon optimization) of the chimeric light chain consisting of the VL of the rat anti-PD-1 antibody and the CL (Lambda chain, GenBank: X62917) of the bovine antibody is shown in SEQ NO: 14.

SEQ ID NO: 10 shows the amino acid sequence of a chimeric heavy chain consisting of the VH of the rat anti-bovine PD-1 antibody and the CH (IgG1 chain, modified from GenBank: X62916) of the bovine antibody. The nucleotide sequence (after codon optimization) of the chimeric heavy chain consisting of the VH of the rat anti-bovine PD-1 antibody and the CH (IgG1 chain, modified from GenBank: X62916) of the bovine antibody is shown in SEQ ID NO: 15.

Amino acid sequences and nucleotide sequences of CLs and CHs of various animals other than rat may be obtained from known databases for use in the present invention.

Amino acid sequences and nucleotide sequences of bovine CL and CH are summarized in the table below.

TABLE

| | | | | | GenBank | | |
|---|---|---|---|---|---|---|---|
| | | | | | Accession | | |
| Species | Ig Domain | | Nucleotide Sequence | Amino Acid Sequence | No. | IMGT Database | Reference |
| Bovine | Bovine Ig | IgG1 | GCCTCCACCACAGCCCCGAAAGTCTACCCTCTGAGTTC | ASTTAPKVYPLSGGGDKSSSTVTLGC | X62916 | http://www. | Symons DB |
| (Scientific | heavy chain | variant 1 | TTGCTGCGGGGACAAGTCCAGCTCCACCGTGACCCTGG | LVSSYMPEPVTVTWNSGALKSGVHTFP | | imgt.org/ | et al., J |
| Name Bos | constant | | GCTGCCTGGTCTCCAGCTACATGCCCGAGCCGGTGACC | AVLQSSGLYSLSSMVTVPGSTSGQTFT | | IMGT | Immogenet, |
| taurus) | region | | GTGACCTGGAACTCGGGTGCCCTGAAGAGCGGCGTGCA | CNVAHPASSTKVDKAVDPTCKPSPCD | | repertoire/ | 14, 273- |
| | (CH1 CH3) | | CACCTTCCCGGCTGTCCTTCAGTCCTCCGGGCTGTACT | CCPPPELPGGPSVFIFPPKPKDTLTISG | | index.php? | 283 (1987) |
| | | | CTCTCAGCAGCATGGTGACCGTGCCCGGCAGCACCTCA | TPEVTCVVVDVGHDDPEVKFSWFVDD | | section= | PMID: |
| | | | GGACAGACCTTCACCTGCAACGTAGCCCACCCGGCCAG | VEVNTATTKPREEQFNSTYRVVSALRI | | LocusGenes& | 3141517 |
| | | | CAGCACCAAGGTGGACAAGGCTGTTGATCCCACATGCA | QHQDWTGGKEFKCKVHNEGLPAPTVRT | | repertoire= | Symons DB |
| | | | AACCATCACCCTGTGACTGTTGCCCACCCCCTGAGTC | ISRTKGPAREPQVYVLAPPQEELSKST | | genetable& | et al., |
| | | | CCCGGAGGACCCTCTGTCTTCATCTTCCCACCGAAACC | VSLTCMVTSFYPDYIAVEWQRNGQPES | | species= | Mol. |
| | | | CAAGGACACCCTCACAATCTGGGGAACGCCCGAGGTCA | EDKYGTTPPQLDADSSYFLYSKLRVDR | | bovine&group= | Immunol., |
| | | | CGTGTGTGGTGGTGGACGTGGGCCACGATGACCCCGAG | NSWQEGDTYTCVVMHEALHNHYTQKS | | IGHC | 841-850 |
| | | | GTGAAGTTCTCCTGGTTCGTGGACGACGTGGAGGTAAA | TSKSAGK* | | | (1989). |
| | | | CACAGCCACGAGGAAGCCGAGAGAGGAGCAGTTCAACA | (SEQ ID NO: 21) | | | PMID: |
| | | | GCACCTACCGCGTGGTCAGCGCCCTGCGCATCCAGCAC | | | | 2513487 |
| | | | CAGGACTGGACTGGAGGAAAGGAGTTCAAGTGCAAGGT | | | | Kacskovica |
| | | | CCACAACGAAGGCCTCCCGGCCCCCATCGTGAGGACCA | | | | L. and |
| | | | TCTCCAGGACCAAAGGGCCGGCCCGGGAGCCGCAGGT | | | | Butler JE., |
| | | | GTATGTCCTGGCCCCACCCCAGGAAGAGCTCAGCAAAA | | | | Mol. |
| | | | GCAGGGTCAGCCTCACCTGCATGGTCACCAGCTTCTAC | | | | Immunol., |
| | | | CCAGACTACATCGCCGTGGAGTGGCAGAGAAAGGGGCA | | | | 33, 189- |
| | | | GCCTGAGTCGGAGGACAAGTACGGCACCACCCCGCCC | | | | 195 (1996). |
| | | | CAGCTGGACGCCGACAGCTCCTACTTCCTGTACAGCAA | | | | PMID: |
| | | | GCTCAGGGTGGACAGGAACAGCTGGCAGGAAGGAGAC | | | | 3649440 |
| | | | ACCTACACGTGTGTGGTGATGCACGAGGCCCTGCACAA | | | | Rabbani H. |
| | | | TCACTACACGCAGAAGTCCACCTCTAAGTCTGCGGGTA | | | | et al., |
| | | | AATGA | | | | Immuno- |
| | | | (SEQ ID NO: 29) | | | | genetics, |
| | | IgG1 | GCCTCCACCACAGCCCCGAAAGTCTACCCTCTGAGTTC | ASTTAPKVYPLSSCCGDKSSSTVTLGC | X16701 | | 46. 326- |
| | | variant 2 | TTGCTGCGGGGACAAGTCCAGCTCCACCGTGACCCTGG | LVSSYMPEPVTVTWNSGALKSGVHTFP | (M25278) | | 331 (1997). |
| | | | GCTGCCTGGTCTCCAGCTACATGCCCGAGCCGGTGACC | AVLQSSGLYSLSSMVTVPGSTSGQTFT | | | PMID: |
| | | | GTGACCTGGAACTCGGGTGCCCTGAAGAGCGGCGTGCA | CNVAHPASSTKVDKAVDPTCKPSPCD | | | 9218535 |
| | | | CACCTTCCCGGCCGTCCTTCAGTCCTCCGGGCTGTACT | CCPPPELPGGPSVFIFPPKPKDTLTISG | | | Saini S.S. |
| | | | CTCTCAGCAGCATGGTGACCGTGCCCGGCAGCACCTCA | TPEVTCVVVDVGHDDPEVKFSWFVDD | | | et al., |
| | | | GGACAGACCTTCACCTGCAACGTAGCCCACCCGGCCAG | VEVNTATTKPREEQFNSTYRVVSALRI | | | Scand J. |
| | | | CAGCACCAAGGTGGACAAGGCTGTTGATCCCACATGCA | QHQDWTGGKEFKCKVHNEGLPAPIVRT | | | Immunol. |
| | | | AACCATCACCCTGTGACTGTTGCCCACCCCCTGAGCTC | ISRTKGPAREPQVYVLAPPQEELSKST | | | 65, 32-8 |
| | | | CCCGGAGGACCCTCTGTCTTCATCTTCCCACCGAAACC | VSLTCMVTSFYPDYIAVEWQRNGQPES | | | (2007). |
| | | | CAAGGACACCCTCACAATCTCGGGAACGCCCGAGGTCA | EDKYGTTPPQLDADSSYFLYSKLRVDR | | | PMID: |
| | | | CGTGTGTGGTGGTGGACGTGGGCCACGATGACCCCGAG | NSWQEGDTYTCVVMHEALHNHYTQKS | | | 17212764 |
| | | | GTGAAGTTCTCCTGGTTCGTGGACGACGTGGAGGTAAA | TSKSAGK* | | | |
| | | | CACAGCCACGACGAAGCCGAGAGAGGAGCAGTTCAACA | (SEQ ID NO: 22) | | | |
| | | | GCACCTACCGCGTGGTCAGCGCCCTGCGCATCCAGCAC | | | | |
| | | | CAGGACTGGACTGGAGGAAAGGAGTTCAAGTGCAAGGT | | | | |
| | | | CCACAACGAAGGCCTCCCGGCCCCCATCGTGAGGACCA | | | | |
| | | | TCTCCAGGACCAAAGGGCCGGCCCGGGAGCCGCAGGT | | | | |
| | | | GTATGTCCTGGCCCCACCCCAGGAAGAGCTCAGCAAAA | | | | |
| | | | GCACGGTCAGCCTCACCTGCATGGTCACCAGCTTCTAC | | | | |
| | | | CCAGACTACATCGCCGTGGAGTGGCAGAGAAACGGGCA | | | | |
| | | | GCCTGAGTCGGAGGACAAGTACGGCACGACCCCGCCC | | | | |
| | | | CAGCTGGACGCCGACAGCTCCTACTTCCTGTACAGCAA | | | | |
| | | | GCTCAGGGTGGACAGGAACAGCTGGCAGGAAGGAGAC | | | | |
| | | | ACCTACACGTGTGTGGTGATGCACGAGGCCCTGCACAA | | | | |
| | | | TCACTACACGCAGAAGTCCACCTCTAAGTCTGCGGGTA | | | | |
| | | | AATGA | | | | |
| | | | (SEQ ID NO: 30) | | | | |
| | | IgG1 | GCCTCCACCACAGCCCCGAAAGTCTACCCTCTGAGTTC | ASTTAPKVYPLSSCCGDKSSSTVTLGC | S82409 | | |
| | | variant 3 | TTGCTGCGGGGACAAGTCCAGCTCCACCGTGACCCTGG | LVSSYMPEPVTVTWNSGALKSGVHTFP | | | |
| | | | GCTGCCTGGTCTCCAGCTACATGCCCGAGCCGGTGACC | AVLQSSGLYSLSSMVTVPGSTSGTQTF | | | |
| | | | GTGACCTGGAACTCGGGTGCCCTGAAGAGCGGCGTGCA | TCNVAHPASSTKVDKAVDPRCKTTCD | | | |
| | | | CACCTTCCCGGCCGTCCTTCAGTCCTCCGGGCTCTACT | CCPPPELPGGPSVFIFPPKPKDTLTISG | | | |
| | | | CTCTCAGCAGCATGGTGACCGTGCCCGGCAGCACCTCA | TPEVTCVVVDVGHDDPEVKFSWFVDD | | | |
| | | | GGAACCCAGACCTTCACCTGCAACGTAGCCCACCCGGC | VEVNTATTKPREEQFNSTYRVVSALRI | | | |
| | | | CAGCAGCACCAAGGTGGACAAGGCTGTTGATCCCAGAT | QHQDWTGGKEFKCKVHNEGLPAPIVRT | | | |
| | | | GCAAAACAACCTGTGACTGTTGCCCACCGCCTGAGCTC | ISRTKGPAREPQVYVLAPPQEELSKST | | | |
| | | | CCTGGAGGACCCTCTGTCTTCATCTTCCCACCGAAACC | VSLTCMVTSFYPDYIAVEWQRNGQPES | | | |
| | | | CAAGGACACCCTCACAATCTCGGGAACGCCCGAGGTCA | EDKYGTTPPQLDADGSYFLYSRLRVDR | | | |
| | | | CGTGTGTGGTGGTGGACGTGGGCCACGATGACCCCGAG | NSWQEGDTYTCVVMHEALHNHYTQKS | | | |
| | | | GTGAAGTTCTCCTGGTTCGTGGACGACGTGGAGGTAAA | TSKSAGK* | | | |
| | | | CACAGCCACGACGAAGCCGAGAGAGGAGCAGTTCAACA | (SEQ ID NO: 23) | | | |
| | | | GCACCTACCGCGTGGTCAGCGCCCTGCGCATCCAGCAC | | | | |
| | | | CAGGACTGGACTGGAGGAAAGGAGTTCAAGTGCAAGGT | | | | |
| | | | CCACAACGAAGGCCTCCCAGCCCCCATCGTGAGGACCA | | | | |
| | | | TCTCCAGGACCAAAGGGCCGGCCCGGGAGCCGCAGGT | | | | |
| | | | GTATGTCCTGGCCCCACCCCAGGAAGAGCTCAGCAAAA | | | | |
| | | | GCACGGTCAGCCTCACCTGCATGGTCACCAGCTTCTAC | | | | |
| | | | CCAGACTACATCGCCGTGGAGTGGCAGAGAAATGGGCA | | | | |
| | | | GCCTGAGTCAGAGGACAAGTACGGCACGACCCCTCCCC | | | | |
| | | | AGCTGGACGCCGACGGCTCCTACTTCCTGTACAGCAGG | | | | |
| | | | CTCAGGGTGGACAGGAACAGCTGGCAGGAAGGAGACA | | | | |
| | | | CCTACACGTGTGTGGTGATGCACGAGGCCCTGCACAAT | | | | |
| | | | CACTACACGCAGAAGTCCACCTCTAAGTCTGCGGGTAA | | | | |
| | | | ATGA | | | | |
| | | | (SEQ ID NO: 31) | | | | |
| | | IgG2 | GCCTCCACCACAGCCCCGAAAGTCTACCCTCTGGCATC | ASTTAPKVYPLASSCGDTSSSTVTLGC | S82407 | | |
| | | variant 1 | CAGCTGCGGAGACACATCCAGCTCCACCGTGACCCTGG | LVSSYMPEPVTVTWNSGALKSGVHTFP | | | |
| | | | GCTGCCTGGTGTCCAGCTACATGCCCGAGCCGGTGACC | AVLQSSGLYSLSSMVTVPASSSGQTFT | | | |
| | | | GTGACCTGGAACTCGGGTGCCCTGAAGAGCGGCGTGCA | CNVAHPASSTKVDKAVGVSIDCSKCHN | | | |
| | | | CACCTTCCCGGCTGTCCTTCAGTCCTCCGGGCTCTACT | QPCVREPSVFIFPPKPKDTLMITGTPEV | | | |
| | | | CTCTCAGCAGCATGGTGACCGTGCCCGCCAGCAGCTCA | TCVVVNVGHDNPEVQFSWFVDDVEVH | | | |
| | | | GGACAGACCTTCACCTGCAACGTAGCCCACCCGGCCAG | TARSKPREEQFNSTYRVVSALPIQHQD | | | |
| | | | CAGCACCAAGGTGGACAAGGCTGTTGGGGTCTCCATTG | WTGGKEFKCKVNNKGLSAPIVRISRSK | | | |
| | | | ACTGCTCCAAGTGTCATAACCAGCCTTGCGTGAGGGAA | GPAREPQVYVLDPPKEELSKSTLSVTC | | | |
| | | | CCATCTGTCTTCATCTTCCCACCGAAACCCAAAGACAC | MVTGFYPEDVAVEWQRNRQTESEDKY | | | |
| | | | CCTGATGATCACAGGAACGCCCGAGGTCACGTGTGTGG | RTTPPQLDTDRSYFLYSKLRVDRNSWQ | | | |
| | | | TGGTGAACGTGGGCCACGATAACCCCGAGGTGCAGTTC | EGDAYTCVVMHEALHNHYMQKSTSKS | | | |
| | | | TCCTGGTTCGTGGATGACGTGGAGGTGCACACGGCCAG | AGK* | | | |
| | | | GTCGAAGCCAAGAGAGGAGCAGTTCAACAGCACGTACC | (SEQ ID NO: 24) | | | |
| | | | GCGTGGTCAGCGCCCTGCCCATCCAGCACCAGGACTGG | | | | |
| | | | ACTGGAGGAAAGGAGTTCAAGTGCAAGGTCAACAACAA | | | | |
| | | | AGGCCTCTGGGCCCCCATCGTGAGGATCATCTCCAGGA | | | | |
| | | | GCAAAGGGCCGGCCCGGGAGCCGCAGGTGTATGTCCT | | | | |
| | | | GGACCCACCCAAGGAAGAGCTCAGCAAAAGCACGCTCA | | | | |
| | | | GCGTCACCTGCATGGTCACCGGCTTCTACCCAGAAGAT | | | | |
| | | | GTAGCCGTGGAGTGGCAGAGAAACCGGCAGACTGAGTC | | | | |
| | | | GGAGGACAAGTACCGCACGACCCCGCCCCAGCTGGAC | | | | |
| | | | ACCGACCGCTCCTACTTCCGTACAGCAAGCTCAGGGT | | | | |
| | | | GGACAGGAACAGCTGGCAGGAAGGAGACGCCTACACG | | | | |
| | | | TGTGTGGTGATGCACGAGGCCCTGCACAATCACTACAT | | | | |
| | | | GCAGAAGTCCACCTCTAAGTCTGCGGGTAAATGA | | | | |
| | | | (SEQ ID NO: 32) | | | | |
| | | IgG2 | GCCTCCACCACAGCCCCGAAAGTCTACCCTCTGAGTTC | ASTTAPKVYPLSSCCGDKSSSTVTLGC | M36946 | | |
| | | variant 2 | TTGCTGCGGGGACAAGTCCAGCTCCACCGTGACCCTGG | LVSSYMPEPVTVTWNSGALKSGVHTFP | (X06703) | | |
| | | | GCTGCCTGGTGTCCAGCTACATGCCCGAGCGGGTGACC | AVLQSSGLYSLSSMVTVPGSTSGQTFT | | | |
| | | | GTGACCTGGAACTCGGGTGCCCTGAAGAGCGGCGTGCA | CNVAHPASSTKVDKAVGVSSDCSKPN | | | |
| | | | CACCTTCCCGGCCGTCCTTCAGTCCTCCGGGCTCTACT | NQHCVREPSVFIFPPKPKDTLMITGTPE | | | |
| | | | CTCTCAGCAGCATGGTGACCGTGCCCGGCAGCACCTCA | VTCVVVNVGHDNPEVQFSWFVDDVEV | | | |
| | | | GGACAGACCTTCACCTGCAACGTAGCCCACCCGGCCAG | HTARTKPREEQFNSTYRVVSALPIQHQ | | | |
| | | | CAGCACCAAGGTGGACAAGGCTGTTGGGGTCTCCAGTG | DWTGGKEFKCKVNIKGLSASIVRIISRSK | | | |
| | | | ACTGCTCCAAGCCTAATAACCAGCATTGCGTGAGGGAA | GPAREPQVYVLDPPKEELSKSTVSVTC | | | |
| | | | CCATCTGTCTTCATCTTCCCACCGAAACCCAAAGACAC | MVIGFYPEDVDVEWQRDRQTESEDKYR | | | |
| | | | CCTGATGATCACAGGAACGCCCGAGGTCACGTGTGTGG | TTPPQLDADRSYFLYSKLRVDRNSWQR | | | |
| | | | TGGTGAACGTGGGCCACGATAACCCCGAGGTGCAGTTC | GDTYTCVVMHEALHNHYMQKSTSKSA | | | |
| | | | TCCTGGTTCGTGGACGACGTGGAGGTGCACACGGCCAG | GK* | | | |
| | | | GACGAAGCCGAGAGAGGAGCAGTTCAACAGCACGTACC | (SEQ ID NO: 25) | | | |
| | | | GCGTGGTCAGCGCCCTGCCCATCCAGCACCAGGACTGG | | | | |
| | | | ACTGGAGGAAAGGAGTTCAAGTGCAAGGTCAACATCAA | | | | |
| | | | AGGCCTCTCGGCCTCCATCGTGAGGATCATCTCCAGGA | | | | |
| | | | GCAAAGGGCCGGCCCGGGAGCCGCAGGTGTATGTCCT | | | | |
| | | | GGACCCACCCAAGGAAGAGCTCAGCAAAAGCACGGTCA | | | | |
| | | | GCGTCACCTGCATGGTCATCGGCTTCTACCCAGAAGAT | | | | |
| | | | GTAGACGTGGAGTGGCAGAGAGACCGGCAGACTGAGTC | | | | |
| | | | GGAGGACAAGTACCGCACGACCCCGCCCCAGCTGGAC | | | | |
| | | | GCCGACCGCTCCTACTTCCTGTACAGCAAGCTCAGGGT | | | | |
| | | | GGACAGGAACAGCTGGCAGAGAGGAGACACCTACACGT | | | | |
| | | | GTGTGGTGATGCACGAGGCCCTGCACAATCACTACATG | | | | |
| | | | CAGAAGTCCACCTCTAAGTCTGCGGGTAAATGA | | | | |
| | | | (SEQ ID NO: 33) | | | | |
| | | IgG2 | GCCTCCACCACAGCCCCGAAAGTCTACCCTCTGAGTTC | ASTTAPVKYPLSSCCGDKSSSGVTLGC | X16702 | | |
| | | variant 3 | TTGCTGCGGGGACAAGTCCAGCTCGGGGGTGACCCTGG | LVSSYPEPVTVTWNSGALKSGVHTFP | (M25279) | | |
| | | | GCTGCCTGGTCTCCAGCTACATGCCCGAGCCGGTGACC | AVLQSSGLYSLSSMVTVPASSSGTQTF | | | |
| | | | GTGACCTGGAACTCGGGTGCCCTGAAGAGCGGCGTGCA | TCNVHAPASSTKVDKAVGVSSDCSKP | | | |
| | | | CACCTTCCCGGCCGTCCTTCAGTCCTCCGGGCTCTACT | NNQHCVREPSVFIFPPKPKDTLMITGTP | | | |
| | | | CTCTCAGCAGCATGGTGACCGTGCCCGCCAGCAGCTCA | EVTCVVVNVGHDNPEVQFSWFVDDVE | | | |
| | | | GGAACCCAGACCTTCACCTGCAACGTAGCCCACCCGGC | VHTARTKPREEQFNSTYRVVSALPIQH | | | |
| | | | CAGCAGCACCAAGGTGGACAAGGCTGTTGGGGTCTCCA | QDWTGGKEFKCKVNIKGLSASIVRIISRS | | | |
| | | | GTGACTGCTCCAAGCCTAATAACCAGCATTGCGTGAGG | KGPAREPQVYVLDPPKEELSKSTVSLT | | | |
| | | | GAACCATCTGTCTTCATCTTCCCACCGAAACCCAAAGA | CMVIGFYPEDVDVEWQRDRQTESEDKY | | | |
| | | | CACCCTGATGATCACAGGAACGCCCGAGGTCACGTGTG | RTTPPQLDADRSYFLYSKLRVDRNSWQ | | | |
| | | | TGGTGGTGAACGTGGGCCACGATAACCCCGAGGTGCAG | RGDTYTCVVMHEALHNHYMQKSTSKS | | | |
| | | | TTCTCCTGGTTCGTGGACGACGTGGAGGTGCACACGGC | AGK* | | | |
| | | | CAGGACGAAGCCGAGAGAGGAGCAGTTCAACAGCACGT | (SEQ ID NO: 26) | | | |
| | | | ACCGCGTGGTCAGCGCCCTGCCCATCCAGCACCAGGAC | | | | |
| | | | TGGACTGGAGGAAAGGAGTTCAAGTGCAAGGTCAACAT | | | | |
| | | | CAAAGGCCTCTCGGCCTCCATCGTGAGGATCATCTCCA | | | | |
| | | | GGAGCAAAGGGCCGGCCCGGGAGCCGCAGGTGTATGT | | | | |
| | | | CCTGGACCCACCCAGGAAGAGCTCAGCAAAAGCACGG | | | | |
| | | | TCAGCCTCACCTGCATGGTCATCGGCTTCTACCCAGAA | | | | |
| | | | GATGTAGACGTGGAGTGGCAGAGAGACCGGCAGACTGA | | | | |
| | | | GTCGGAGGACAAGTACCGCACGACCCCGCCCCAGCTG | | | | |
| | | | GACGCCGACCGCTCCTACTTCCTGTACAGCAAGCTCAG | | | | |
| | | | GGTGGACAGGAACAGCTGGCAGAGAGGAGACACCTAC | | | | |
| | | | ACGTGTGTGGTGATGCACGAGGCCCTGCACAATCACTA | | | | |
| | | | CATGCAGAAGTCCACCTCTAAGTCTGCGGGTAAATGA | | | | |
| | | | (SEQ ID NO: 34) | | | | |
| | | IgG3 | GCCTCCACCACAGCCCCGAAAGTCTACCCTCTGGCATC | ASTTAPKVYPLASSCGDTSSSTVTLGC | U63638 | | |
| | | variant 1 | CAGCTGCGGAGACACATCCAGCTCCACCGTGACCCTGG | LVSSYMPEPVTVTWNSGALKSGVHTFP | | | |
| | | | GCTGCCTGGTCTCCAGCTACATGCCCGAGGGGGTGACC | AVRQSSGLYSLSSMVTVPASSSETQTF | | | |
| | | | GTGACCTGGAACTCGGGTGCCCTGAAGAGCGGCGTGCA | TCNVAHPASSTKVDKAVTARRPVPTTP | | | |
| | | | CACCTTCCCGGCCGTCCGGCAGTCCTCTGGGCTGTACT | KTTIPPGKPTTPKSEVEKTPCQCSKCP | | | |
| | | | CTCTCAGCAGCATGGTGACTGTGCCCGCCAGCAGCTCA | EPLGGLSVFIFPPKPKDTLTISGTPEVT | | | |
| | | | GAAACCCAGACCTTCACCTGCAACGTAGCCCACCCGGC | CVVVDVGQDDPEVQFSWFVDDVEVHT | | | |
| | | | CAGCAGCACCAAGGTGGACAAGGCTGTCACTGCAAGGC | ARTKPREEQFNSTYRVVSALRIQHQDW | | | |
| | | | GTCCAGTCCCGACGACGCCAAAGACAACTATCCCTCCT | LQGKEFKCKVNNKGLPAPIVRTISRTKG | | | |
| | | | GGAAAACCCACAACCCCAAAGTCTGAAGTTGAAAAGAC | QAREPQVYVLAPPREELSKSTLSLTCLI | | | |
| | | | ACCCTGCCAGTGTTCCAAATGCCCAGAACCTCTGGGAG | TGFYPEEIDVEWQRNGQPESEDKYHITT | | | |
| | | | GACTGTCTGTCTTCATCTTCCCACCGAAACCCAAGGAC | APQLDADGSYFLYSKLRVNKSSWQEG | | | |
| | | | ACCCTCACAATCTCGGGAACGCCCGAGGTCAGGTGTGT | DHYTCAVMHEALRNHYKEKSISRSPGK | | | |
| | | | GGTGGTGGACGTGGGCCAGGATGACCCCGAGGTGCAG | * | | | |
| | | | TTCTCCTGGTTCGTGGACGACGTGGAGGTGCACACGGC | (SEQ ID NO: 27) | | | |
| | | | CAGGACGAAGCCGAGAGAGGAGCAGTTCAACAGCACCT | | | | |
| | | | ACCGCGTGGTCAGCGCCCTGCGCATCCAGCACCAGGA | | | | |
| | | | CTGGCTGCAGGGAAAGGAGTTCAAGTGCAAGGTCAACA | | | | |
| | | | ACAAAGGCCTCCCGGCCCCCATTGTGAGGACCATCTCC | | | | |
| | | | AGGACCAAAGGGCAGGCCCGGGAGCCGCAGGTGTATG | | | | |
| | | | TCCTGGCCCCACCCCGGGAAGAGCTCAGCAAAGCACG | | | | |
| | | | CTCAGCCTCACCTGCCTGATCACCGGTTTCTACCCAGA | | | | |
| | | | AGAGATAGACGTGGAGTGGCAGAGAAATGGGCAGCCTG | | | | |
| | | | AGTCGGAGGACAAGTACCACACGACCGCACCCCAGCTG | | | | |
| | | | GATGCTGACGGCTCCTACTTCCTGTACAGCAAGCTCAG | | | | |
| | | | GGTGAACAAGAGCAGCTGGCAGGAAGGAGACCACTACA | | | | |
| | | | CGTGTGCAGTGATGCACGAAGCTTTACGGAATCACTAC | | | | |
| | | | AAAGAGAAGTCCATCTCGAGGTCTCCGGGTAAATGA | | | | |
| | | | (SEQ ID NO: 35) | | | | |
| | | IgG3 | GCCTCCACCACAGCCCCGAAAGTCTACCCTCTGGCATC | ASTTAPKVYPLASRCGDTSSSTVTLGC | U63639 | | |
| | | variant 2 | CCGCTGCGGAGACACATCCAGCTCCACCGTGACCCTGG | LVSSYMPEPVTVTWNSGALKSGVHTFP | | | |
| | | | GCTGCCTGGTCTCCAGCTACATGCCCGAGCCGGTGACC | AVLQSSGLYSLSSMVTVPASTSETQTF | | | |
| | | | GTGACCTGGAACTCGGGTGCCCTGAAGAGTGGCGTGCA | TCNVAHPASSTKVDKAVTARRPVPTTP | | | |
| | | | CACCTTCCCGGCCGTCCTTCAGTCCTCCGGGCTGTACT | KTTIPPGKPTTQESEVEKTPCQCSKCP | | | |
| | | | CTCTCAGCAGCATGGTGACCGTGCCCGCCAGCACCTCA | EPLGGLSVFIFPPKPKDTLTISGTPEVT | | | |
| | | | GAAACCCAGACCTTCACCTGCAACGTAGCCCACCCGGC | CVVVDVGQDDPEVQFSWFVDDVEVHT | | | |
| | | | CAGCAGCACCAAGGTGGACAAGGCTGTCACTGCAAGGC | ARTKPREEQFNSTYRVVSALRIQHQDW | | | |
| | | | GTCCAGTCCCGACGACGCCAAAGACAACCATCCCTCCT | LQGKEFKCKVNNKGLPAPIVRTISRTKG | | | |
| | | | GGAAAACCCACAACCCAGGAGTCTGAAGTTGAAAAGAC | QAREPQVYVLAPPPREELSKSTLSLTCLI | | | |
| | | | ACCCTGCCAGTGTTCCAAATGCCCAGAACCTCTGGGAG | TGFYPEEIDVEWQRNGQPESEDKYHTT | | | |
| | | | GACTGTCTGTCTTGATCTTCCCACCGAAACCCAAGGAC | APQLDADGSYFLYSRLRVNKSSWQEG | | | |
| | | | ACCCTCACAATCTCGGGAACGCCCGAGGTCACGTGTGT | DYYTCAVMHEALRNHYKEKSISRSPGK | | | |
| | | | GGTGGTGGACGTGGGCCAGGATGACCCCGAGGTGCAG | * | | | |
| | | | TTCTCCTGGTTGGTGGACGACGTGGAGGTGCACACGGC | (SEQ ID NO: 28) | | | |
| | | | CAGGACGAAGCCGAGAGAGGAGCAGTTCAACAGCACCT | | | | |
| | | | ACCGCGTGGTCAGCGCCCTGCGCATCCAGCACCAGGA | | | | |
| | | | CTGGCTGCAGGGAAAGGAGTTCAAGTGCAAGGTCAACA | | | | |
| | | | ACAAAGGCCTCCCGGCCCCCATTGTGAGGACCATCTCC | | | | |
| | | | AGGACCAAAGGGCAGGCCCGGGAGCCGCAGGTGTATG | | | | |
| | | | TCCTGGCCCCACCCCGGGAAGAGCTCAGCAAAAGCACG | | | | |
| | | | CTCAGCCTCACCTGCCTGATCACCGGTTTCTACCCAGA | | | | |
| | | | AGAGATAGACGTGGAGTGGCAGAGAAATGGGCAGCCTG | | | | |
| | | | AGTCGGAGGACAAGTACCACACGACCGCACCCCAGCTG | | | | |
| | | | GATGCTGACGGCTCCTACTTCCTGTACAGCAGGCTCAG | | | | |
| | | | GGTGAACAAGAGCAGCTGGCAGGAAGGAGACCACTACA | | | | |
| | | | CGTGTGCAGTGATGCATGAAGCTTTACGGAATCACTAC | | | | |
| | | | AAAGAGAAGTCCATCTCGAGGTCTCCGGGTAAATGA | | | | |
| | | | (SEQ ID NO: 36) | | | | |
| | Bovine Ig | Ig lambda | CAGCCCAAGTCCCCACCCTCGGTCACCCTGTTCCCGCC | QPKSPPSVTLFPPSTEELNGNKATLVC | X62917 | Not | Chen L. |
| | light chain | | CTCCACGGAGGAGCTCAACGGCAACAAGGCCACCCTG | LISDFYPGSVTVVWKADGSTITRNVETT | | registered | et al., |
| | constant | | GTGTGTCTCATCAGCGACTTCTACCCGGGTAGCGTGAC | RASKQSNSKYAASSYLSLTSSDWKSKG | | | Vet, |
| | region (CL) | | CGTGGTCTGGAAGGCAGACGGCAGCACCATCACCCGCA | SYSCEVTHEGSTVTKTVKPSECS* | | | Immunol. |
| | | | ACGTGGAGACCACCCGGGCCTCCAAACAGAGCAACAG | | | | Immuno- |
| | | | CAAGTACGCGGCCAGCAGCTACCTGAGCCTGACGAGCA | | | | pathol. |
| | | | GCGACTGGAAATCGAAAGGCAGTTACAGCTGCGAGGTC | | | | 124, 284- |
| | | | ACGCACGAGGGGAGCACCGTGACGAAGACAGTGAAGC | | | | 294 (2008). |
| | | | CCTCAGAGTGTTCTTAG | | | | PMID: |
| | | | (SEQ ID NO: 7) | | | | 1853886 |

Amino acid sequences and nucleotide sequences of ovine, water buffalo and human CL and CH are summarized in the table below.

TABLE

| | | | | | GenBank | | |
|---|---|---|---|---|---|---|---|
| | | | | | Accession | | |
| Species | Ig Domain | | Nucleotide Sequence | Amino Acid Sequence | No. | IMGT Database | Reference |
| Ovine | Ovine Ig | IgG1 | GCCTCAACAACACCGGCGAAAGTCTACCCTCTGACT | ASTTPPKVYPLTSCCGDTSSSIVTLGC | X69797 | http://www. | Dufour V. |
| (Scientific | heavy chain | | TCTTGCTGCGGGGACACGTCCAGCTCCATCGTGACC | LVSSYMPEPVTVWTNSGALTSGVHTF | | IMGT | et al., |
| Name Ovis | constant | | CTGGGCTGCCTGGTCTCCAGCTATATGCCCGAGGGG | PAILQSSGLYSLSSVVTVPASTSGAQT | | repertoire | J. |
| aries) | region | | GTGACCGTGACCTGGAACTCTGGTGCCCTGACCAGC | FICNVAHPASSTKVDKRVEPGCPDPCK | | indes.php? | Immunol., |
| | (CH1 CH3) | | GGCGTGCACACCTTCCCGGCCATCCTGCAGTCCTCC | HCRCPPPELPGGPSVFIFPPKPKDTLTI | | section= | 156, 2163- |
| | | | GGGCTCTACTCTCTCAGCAGCGTGGTGACCGTGCCG | SGTPEVTCVVVDVGQDDPEVQFSWFV | | LocusGenes& | 2170 |
| | | | GGGCTCTACTCTCTCAGCAGCGTGGTGACCGTGCCG | DNVEVRTARTKPREEQFNSTFRVVSAL | | repertoire= | (1996). |
| | | | GCCAGCACCTCAGGAGCCCAGACCTTCATGTGCAAC | PIQHQDWTGGKEFKCKVHNEALPAPIV | | genetable& | PMID: |
| | | | GTAGGCGACCCGGCCAGCAGCACCAAGTGGACAAG | RTISRTKGQAREPQVYVLAPPQEESK | | species= | 8690905 |
| | | | CGTGTTGAGCCCGGATGCCCGGACCCATGCAAACAT | STLSVTCLVTFGYPDYIAVEWQKNGQP | | sheep&group= | |
| | | | TGCCGATGCCCACCCCCTGAGCTCCCCGGAGGACC | ESEDKYGTTTSQLDADGSYFLYSRLRV | | IGHC | |
| | | | GTCTGTCTTCATCTTCCCACCGAAACCCAAGGACAC | DKNSWQEGDTYACVVMHEALHNHYTQ | | | |
| | | | CCTTACAATCTCTGGAACGCCCGAGGTCACGTGTGT | KSISKPPGK* | | | |
| | | | GGTGGTGGACGTGGGCCAGGATGACCCCGAGGTGC | (SEQ ID NO: 37) | | | |
| | | | AGTTCTCCTGGTTCGTGGACAACGTGGAGGTGCGCA | | | | |
| | | | CGGCCAGGACAAAGCCGAGAGAGGAGCAGTTCAACA | | | | |
| | | | GCACCTTCCGCGTGGTCAGCGCCCTGCCCATCCAGC | | | | |
| | | | ACCAAGACTGGACTGGAGGAAAGGAGTTCAAGTGCA | | | | |
| | | | AGGTCCACAACGAAGCCCTCCCGGCCCCCATCGTGA | | | | |
| | | | GGACCATCTCCAGGACCAAAGGGCAGGCCCGGGAG | | | | |
| | | | CCGCAGGTGTACGTCCTGGCCCCACCCCAGGAAGAG | | | | |
| | | | CTCAGCAAAAGCACGCTCAGCGTCACCTGCCTGGTC | | | | |
| | | | ACCGGCTTCTACCCAGACTACATCGCCGTGGAGTGG | | | | |
| | | | CAGAAAAATGGGCAGCCTGAGTCGGAGGACAAGTAC | | | | |
| | | | GGCACGACCACATCCCAGCTGGACGCCGACGGCTCC | | | | |
| | | | TACTTCCTGTACAGCAGGCTCAGGGTGGACAAGAAC | | | | |
| | | | AGCTGGCAAGAAGGAGACACCTACGCGTGTGTGGTG | | | | |
| | | | ATGCACGAGGCTCTGCACAACCACTACACACAGAAG | | | | |
| | | | TCGATCTCTAAGCCTCCGGGTAAATGA | | | | |
| | | | (SEQ ID NO: 38) | | | | |
| | | IgG2 | GCCTCCACCACAGCCCCGAAAGTCTACCCTCTGACT | ASTTAPKVYPLTSCCGDTSSSSSIVTL | X70983 | | Clarkson |
| | | | TCTTGCTGCGGGGACACGTCCAGCTCCAGOTCCATC | CLVSSYMPEPVTVTWNSGALTSGVH | | | C.A. et |
| | | | GTGACCCTGGGCTGCCTGGTCTCCAGCTATATGCCC | TFPAILQSSGLYSLSSVVTVPASTSGA | | | al., Mol. |
| | | | GAGCCGGTGACCGTGACCTGGAACTCTGGTGCCCTG | QTFICNVAHPASSAKVDKRVGISSDYS | | | Immunol, |
| | | | ACCAGCGGCGTGCACACCTTCCCGGCCATCCTGCAG | KCSKPPCVSRPSVFIFPPKPKDSLMITG | | | 30, 1195- |
| | | | TCCTCCGGGCTCTACTCTCTCAGCAGCGTGGTGACC | TPEVTCVVVDVGQGDPEVQFSWFVDN | | | 1204 |
| | | | GTGCCGGCCAGCACCTCAGGAGCCCAGACCTTCATC | VEVRTARTKPREEQFNSTFRVVSALPI | | | (1993). |
| | | | TGCAACGTAGCCCACCCGGCCAGCAGCGCCAAGGTG | QHDHWTGGKEFKCKVHSKGLPAPIVRT | | | PMID: |
| | | | GACAAGCGTGTTGGGATCTCCAGTGACTACTCCAAG | ISRAKGQAREPQVYVLAPPQEELSKST | | | 8413324 |
| | | | TGTTCTAAACCGCCTTGCGTGAGCCGACCGTCTGTC | LSVTCLVTGFYPDYIAVEWQRARQPES | | | |
| | | | TTCATCTTCCCCCCGAAACCCAAGGACAGCCTCATG | EDKYGTTTSQLDADGSYFLYSRLRVDK | | | |
| | | | ATCACAGGAACGCCCGAGCTCACGTGTGTGGTGGTG | SSWQRGDTYACVVMHEALHNHYTQKS | | | |
| | | | GACGTGGGCCAGGGTGACCCCGAGGTGCAGTTCTCC | ISKPPGK* | | | |
| | | | TGGTTCGTGGACAACGTGGAGGTGCGCACGGCCAGG | (SEQ ID NO: 39) | | | |
| | | | ACAAAGCCGAGAGAGGAGCAGTTCAACAGCACCTTC | | | | |
| | | | CGCGTGGTCAGCGCCCTGCCCATCCAGCACGACCAC | | | | |
| | | | TGGACTGGAGGAAAGGAGTTCAAGTGCAAGGTCCAC | | | | |
| | | | TAGCAAAGGCCTCCCGGCCCCCATCGTGAGGACCATC | | | | |
| | | | TCCAGGGCCAAAGGGCAGGCCCGGGAGCCGCAGGT | | | | |
| | | | GTACGTCCTGGCCCCACCCCAGGAAGAGCTCAGCAA | | | | |
| | | | AAGCACGCTCAGCGTCACCTGCCTGGTCACCGGCTT | | | | |
| | | | CTACCCAGACTACATCGCCGTGGAGTGGCAGAGAGC | | | | |
| | | | GCGGCAGCCTGAGTCGGAGGACAAGTACGGCACGAC | | | | |
| | | | CACATCCCAGCTGGACGCCGACGGCTCCTACTTCCT | | | | |
| | | | GTACAGCAGGCTCAGGGTGGACAAGAGCAGCTGGCA | | | | |
| | | | AAGAGGAGACACCTACGCCTGTGTCGTGATGCACGA | | | | |
| | | | GGCTCTGCACAACCACTACACACAGAAGTCGATCTC | | | | |
| | | | TAAGCCTCCGGGTAAATGA | | | | |
| | | | (SEQ ID NO: 40) | | | | |
| | Ovine Ig | Ig kappa | CCATCCGTCTTCCTCTTCAAACCATCTGAGGAACAG | PSVFLFKPSEEQLRTGTVSVVCLVNDF | X54110 | Not | Jenne C.N. |
| | light chain | (CK) | CTGAGGACCCGAACTGTCTCTGTCGTGTGCTTGGTG | YPKDINVKVKVDGVTQNSNFQNSFTDQ | | registered | et al., |
| | constant | | AATGATTTCTACCCCAAAGATATCAATGTCAAGGTGA | DSKKSTYSLSSTLTLSSSEYQSHNAYA | | | Dev. |
| | region | | AAGTGGATGGGGTTACCCAGAACAGCAACTTCCACA | CEVSHKSLPTALVKSFNKNEC* | | | Comp. |
| | | | ACAGCTTCACAGACCAGGACAGCAAGAAAAGCACCT | (SEQ ID NO: 41) | | | Immunol. |
| | | | ACAGCCTCAGCAGCACCCTCACACTGTCCAGCTCAG | | | | 30 (1-2). |
| | | | AGTACCAGAGCCATAACGCCTATGCGTGTGAGGTCA | | | | 165-174 |
| | | | GCCACAAGAGCCTGCCCACCGCCCTCGTCAAGAGCT | | | | (2006). |
| | | | TCAATAAGAATGAATGTTAG | | | | PMID: |
| | | | (SEQ ID NO: 42) | | | | 16083958 |
| | | Ig lambda | GGTCAGCCCAAGTCCGCACCCTCGGTCACCCTGTTC | GQPKSAPSVTLFPPSTEELSTNKATVV | AY734681 | | |
| | | (CL) | CCGCCTTCCACGGAGCAGCTCAGTACCAACAAGGCC | CLINDFYPGSVNVVWKADGSTINQNVK | | | |
| | | | ACCGTGGTCTGTCTCATCAACGACTTCTACCCGGGT | TTQASKQSNSKYAASSYLTLTGSEWKS | | | |
| | | | AGCGTGAACGTGGTCTGGAAGGCAGATGGCAGCACC | KSSYTCEVTHEGSTVTKTVKPSECS* | | | |
| | | | ATCAATCAGAACGTGAAGACCACCCAGGCCTCCAAA | (SEQ ID NO: 43) | | | |
| | | | CAGAGCAACAGCAAGTACGCGGCCAGCAGCTACCTG | | | | |
| | | | ACCCTGACGGGCAGCGAGTGGAAGTCTAAGAGCAGT | | | | |
| | | | TACACCTGCGAGGTCACGCACGAGGGGAGCACCGTG | | | | |
| | | | ACGAAGACAGTGAAGCCCTCAGAGTGTTCTTAG | | | | |
| | | | (SEQ ID NO: 44) | | | | |
| Water | Water | IgG1? | GAGCGGCGTGCACACCTTCCCGGCCGTCCTTCAGTCC | SGVHTFPAVLQSSGLYSLSSTVTAPAS | NW_ | Not | None |
| buffalo | buffalo Ig | | TCCGGGCTCTACTCTCTCAGCAGCACGGTGACCGCGC | ATKSQTFTCNVAHPASSTKVDKAVVP | 005690903 | registered | |
| (Scientific | heavy chain | | CCGCCAGCGCCACAAAAAGCCAGACCTTCACCTGCAA | PCRPKPCDCCPPPELPGGPSVFIFPPK | | | |
| Name: | constant | | GGTAGCCCACCCGGCCAGCAGCACCAAGGTGGACAAG | PKDTLTISGTPEVTCVVVDVGHDDPEV | | | |
| Bubalus | region | | GCTGTTGTTCCCCCATGCAGACCGAAACCCTGTGATTG | KFSWFVDDVEVNTARTKPREEQFNSTY | | | |
| bablis) | (CH1 CH3) | | CTGCCCACCCCCTGAGCTCCCCGGAGGACCCTCTGTC | RVVSALPIQHNDWTGGKEFKCKVYNEG | | | |
| | | | TTCATCTTCCCACCAAAACCCAAGGACACCCTCACAAT | LPAPIVRTISRTKGQAREPQVYVLAPP | | | |
| | | | CTCTGGAACTCCTGAGGTCACGTGTGTGGTGGTGGAC | QDELSKSTVSITCMVTGFYPDYIAVEW | | | |
| | | | GTGGGCCACGATGACCCCGAGGTGAAGTTCTCCTGGT | QKDGQPESEDKYGTTPPQLDSDGSYF | | | |
| | | | TCGTGGACGATGTGGAGGTAAACACAGCCAGGACGAA | LYSRLRVNKNSWQEGGAYTCVVMHE | | | |
| | | | GCCAAGAGAGGAGCAGTTCAACAGCACCTACCGCGTG | (SEQ ID NO: 45) | | | |
| | | | GTCAGCGCCCTGCCCATCCAGCACAACGACTGGACTG | | | | |
| | | | GAGGAAAGGAGTTCAAGTGCAAGGTCTACAATGAAGGC | | | | |
| | | | CTCCCAGCCCCCATCGTGAGCACCATCTCCAGCACCA | | | | |
| | | | AAGGGCAGGCCCGGGAGCCGCAGGTGTACGTCCTGGC | | | | |
| | | | CCCACCCCAGGACGAGCTCAGCAAAAGCACGGTCAGC | | | | |
| | | | ATCACTTGCATGGTCACTGGCTTCTACCCAGACTACAT | | | | |
| | | | CGCCGTAGAGTCGCAGAAAGATGGGCAGCCTGAGTCA | | | | |
| | | | GAGGACAAATATGGCACGACCCCGCCCCAGCTGGACA | | | | |
| | | | GCGATGGCTCCTACTTCCTGTACAGCAGGCTCAGGGT | | | | |
| | | | GAACAAGAACAGCTGGCAAGAAGGAGGCGCCTACACG | | | | |
| | | | TGTGTAGTGATGCATGAGGC | | | | |
| | | | (SEQ ID NO: 46) | | | | |
| | | IgG2? | GCCTCCATCACAGCCCCGAAAGTCTACCCTCTGACTTC | ASITAPKVYPLTSCRGETSSSTVTLGC | NW_ | | |
| | | | TTGCCGCGGGGAAACGTCCACCTCCACCGTGACCCTG | LVSSYMPEPVTVTWNSGALKSGVHTF | 005766143 | | |
| | | | GGCTGCCTGGTCTCCAGCTACATGCCCGAGCCGGTGA | TAVLQSSGLYSLSSTVTAPASATKSQT | | | |
| | | | CCGTGACCTGGAACTCGGGTGCCCTGAAGAGCGGCGT | FTCNVAHPASSTKVDTAVGFSSDCCK | | | |
| | | | GCACACCTTCCCCGCCGTCCTTCAGTCCTCTGGGCTC | FPKPCVRGPSVFIFPPKPKDTLMITGNP | | | |
| | | | TACTCTCTCAGCAGCACGGTGACCGCGCCCGCCAGCG | EVTCVVVDVGRDNPEVQFSWFVGDVE | | | |
| | | | CCACAAAAAGCCAGACCTTCACCTGCAACGTAGCCCAC | VHTGRSKPREEQFNSTYRVVSTLPIQH | | | |
| | | | CCGGCCAGCAGCACCAAGGTGGACACGGCTGTTGGGT | NDWTGGKEFKCKVNNKGLPAPIVRTIS | | | |
| | | | TCTCCAGTGACTGCTCCAAGTTTCCTAAGCCTTGTGTG | FTKGQAREPQVYVLAPPQEELSKSTVS | | | |
| | | | AGGGGACCATCTGTCTTCATCTTCCCGCCGAAACCCAA | VTCMVTGFYPDYIAVEWHRDRQAESED | | | |
| | | | AGACACCCTGATGATCACAGGAAATCCCGAGGTCACAT | KYRTTPPQLDSDGSYFLYSRLKVNKNS | | | |
| | | | GTGTGGTGGTGGACGTGGGCCGGGATAACCCCGAGGT | GQEGGAYTCVVMHE | | | |
| | | | GCACTTCTCCTGGTTCGTGGGTGATCTGGAGGTGCAC | (SEQ ID NO: 47) | | | |
| | | | ACGGGCAGGTCGAAGCCGAGAGAGGAGCAGTTCAACA | | | | |
| | | | GCACCTACCGCGTGGTCAGCACCCTGCCCATCCAGCA | | | | |
| | | | CAATGACTGGACTGGAGGAAAGGAGTTCAAGTGCAAG | | | | |
| | | | GTCAACAACAAAGGCCTCCCAGCCCCCATCGTGAGGA | | | | |
| | | | CCATCTCCAGGACCAAAGGGCAGGCCCGCGAGCCGCA | | | | |
| | | | GGTGTACGTCCTGGCCCCACCCCAGGAAGAGCTCAGC | | | | |
| | | | AAAAGCACGGTCAGCGTCACTTGCATGGTCACTGGCTT | | | | |
| | | | CTACCCAGACTACATCGCCGTAGAGTGGCATAGAGACC | | | | |
| | | | GGCAGGCTGAGTCGGAGGACAAGTACCGCACGACCCC | | | | |
| | | | GCCCCAGCTGGACAGCGATGGCTCCTACTTCCTGTAC | | | | |
| | | | AGCAGGCTCAAGGTGAACAAGAACAGCTGGCAAGAAG | | | | |
| | | | GAGGCGCCTACACGTGTGTACTGATGCATGAGGC | | | | |
| | | | (SEQ ID NO: 48) | | | | |
| | | IgG3? | GCCTCCACCACAGCCCCGAAAGTCTACCCTCTGGCAT | ASTTAPKVYPLASSCGDTSSSTVTLGC | NW_ | | |
| | | | CCAGCTGCGGGGACACGTCCAGCTCCACCGTGACCCT | LVSSYMPEPVTVTWNSGALKNGVHTF | 005784206 | | |
| | | | GGGCTGCCTGGTCTCCAGCTACATGCCCGAGCCGGTG | PAVRQSSGLYSLSSMVTMPTSTAGTQ | | | |
| | | | ACCGTGACCTGGAACTCGGGTGCCCTGAAGAACGGCG | TFTCNVAHPASSTKVDTAVTARHPVP | | | |
| | | | TGCACACCTTCCCGGCCGTCCGGCAGTCCTCCGGGCT | KTPETPIHPVKPPTQEPRDEKTPCQCP | | | |
| | | | CTACTCTCTCAGCAGCATGGTGAGCATGCCCACCAGGA | KCPEPLGGLSVFIFPPKPKDTLTISGTP | | | |
| | | | CCGCAGGAACCCAGACCTTCACCTGCAACGTAGCCCA | EVTCVVVDVGQDDPEVQFSWFVDDVE | | | |
| | | | CCCGGCCAGCAGCACCAAGGTGGACACGGCTGTCACT | VHTARMKPREEQFNSTYRVVSALPIQH | | | |
| | | | GCAAGGCATCCCGTCCCGAAGACACCAGAGACACCTA | QDWLREKEFKCKVNNKGLPAPIVRTISR | | | |
| | | | TCCATCCTTAAAACCCCCAACCCAGGAGCCCAGAGAT | TKGQAREPQVYVLAPPREELSKSTLSL | | | |
| | | | GAAAAGACACCCTGCCAGTGTCCCAAATGCCCAGAACC | TCLITGFYPEEVDVEWQRNGQPESEDK | | | |
| | | | TCTGGGAGGACTGTCTGTCTTCATCTTCCCACCGAAAC | YHTTPPQLDADGSYFLYSRLRVNRSSW | | | |
| | | | CCAAGGACACCCTCACAATCTCTGGAACGCCCGAGGT | QEGDHYTCAVMHEALRNHYKEKPISRS | | | |
| | | | CACGTGTGTGGTGGTGGACGTGGGCCAGGATGACCCC | PGK* | | | |
| | | | GAAGTGCAGTTCTCCTGGTTCGTGGATGACGTGGAGG | (SEQ ID NO: 49) | | | |
| | | | TGCAGACAGCCAGGATGAAGCCAAGAGAGGAGCAGTT | | | | |
| | | | CAACAGCACCTACCGCGTGGTCAGCGCCCTGCCCATC | | | | |
| | | | CAGCACCAGGACTGGCTGCGGGAAAAGGAGTTCAAGT | | | | |
| | | | GCAAGGTCAACAACAAAGGCCTCCCGGCCCCCATCGT | | | | |
| | | | GAGGACCATCTCCAGGACCAAAGGGCAGGCCCGGGAG | | | | |
| | | | CCACAGGTGTATGTCCTGGCCCCACCCCGGGAAGAGC | | | | |
| | | | TCAGCAAAAGCACGCTCAGCCTCACCTGCCTAATCACC | | | | |
| | | | GGCTTCTACCCAGAAGAGGTAGACGTGGAGTGGCAGA | | | | |
| | | | GAAATGGGCAGCCTGAGTCAGAGGACAAGTACCACAC | | | | |
| | | | GACCCCACCCCAGCTGGACGCTGACGGCTCCTACTTC | | | | |
| | | | CTGTACAGCAGGCTCAGGGTGAACAGGAGCAGCTGGC | | | | |
| | | | AGGAAGGAGACCACTACACGTGTCCAGTGATGCATGAA | | | | |
| | | | GCTTTACGGAATCACTACAAAGAGAAGCCCATCTCGAG | | | | |
| | | | GTCTCCGGGTAAATGA | | | | |
| | | | (SEQ ID NO: 50) | | | | |
| | Water | Ig | CAGCCCAAGTCCGCACCCTCAGTCACCCTGTTCCCAC | QPKSAPSVTLFPPSTEELSANKATLVC | NW_ | Not | None |
| | buffalo Ig | lambda? | CCTCCACGGAGGAGCTCAGCGCCAACAAGGCCACCCT | LISDFYPGSMTVARKADGSTITRNVETT | 0055690786 | registered | |
| | light chain | | GGTGTGTCTCATCAGCGACTTCTACCCGGGTAGCATGA | RASKQSNSKYAASSYLSLTGSEWKSKG | | | |
| | constant | | CCGTGGCCAGGAAGGCAGACGGCAGCACCATCACCCG | SYSCEVTHEGSTVTKTVKPSECS* | | | |
| | region (CL) | | GAACGTGGAGACCACCCGGGCCTCCAAACAGAGCAAC | (SEQ ID NO: 51) | | | |
| | | | AGCAAGTACGCCGCCAGCAGCTACCTGAGCCTGACGG | | | | |
| | | | GCAGCGAGTGGAAATCGAAAGGCAGTTACAGCTGCGA | | | | |
| | | | GGTCACGCACGAGGGGAGCACCGTGACAAAGACAGTG | | | | |
| | | | AAGCCCTCAGAGTGTTCTTAC | | | | |
| | | | (SEQ ID NO: 52) | | | | |
| Human | Human Ig | IgG4 | GAGTCCAAATATGGTCCCCCATGCCCATCATGCCCA | ESKYGPPCPSCPAPEFLGGPSVFLFPP | K01316 | http://www. | Ellison J. |
| (Scientific | heavy chain | variant | GCACCTGAGTTCCTGGGGGGACCATCAGTCTTCCTG | KPKDTLMISRTPEVTCVVVDVSQEDPE | | IMGT | et al., |
| Name: Homo | constant | 1 | TTCCCCCCAAAACCCAAGGACACTCTCATGATCTCC | VQGNWYVDGVEVHNAKTKPREEQFNS | | repertoire | DNA, 1, |
| sapiens) | region | | CGGACCCCTGAGGTCACGTGCGTGGTGGTGGACCTG | TYRVVSVLTVLHQDWLNGKEYKCKVS | | indes.php? | 11-18 |
| | (CH1 CH3) | | AGCCAGGAAGACCCCGAGGTCCAGTTCAACTGGTAC | NKGLPSSIEKTISKAKGQPREPQVYTLP | | section= | (1981). |
| | | | GTGGATGGCGTGGAGGTGCATAATGCCAAGACAAAG | PSQEEMTKNQVSLTCLVKGFYPSDIAV | | LocusGenes& | PMID: |
| | | | CCGCGGGAGGAGCAGTTCAACAGCACGTACCGTGTG | EWESNGQPENNYKTTPPVLDSDGSFFL | | repertoire= | 6299662 |
| | | | GTCAGCGTCCTCACCGTCCTGCACCAGGACTGGCTG | YSRLTVDKSRWQEGNVFSCSVMHEAL | | genetable& | |
| | | | AACGGCAAGGAGTACAAGTGCAAGGTCTCCAACAAA | HNHYTQKSLSLSLGK* | | species= | |
| | | | GGCCTCCCGTCCTCCATCGAGAAAACCATCTCCAAA | (SEQ ID NO: 53) | | human&group= | |
| | | | GCCAAAGGGCAGCCCCGAGAGCCACAGGTGTACACC | | | IGHC | |
| | | | CTGCCCCCATCCCAGGAGGAGATGACCAAGAACCAG | | | | |
| | | | GTCAGCCTGACCTGCCTGGTCAAAGGCTTCTACCCC | | | | |
| | | | AGCGACATCGCCGTGGAGTGGGAGAGCAATGGGCAG | | | | |
| | | | CCCGAGAACAACTACAAGACCACGCCTCCCGTGCTG | | | | |
| | | | GACTCCGACGGCTCCTTCTTCCTCTACAGCAGGCTA | | | | |
| | | | ACCGTGGACAAGAGCAGGTGGCAGGAGGGGAATGTC | | | | |
| | | | TTCTCATGCTCCGTGATGCATGAGGCTCTGCACAAC | | | | |
| | | | CACTACACACAGAAGAGCCTCTCCCTGTCTCTGGGT | | | | |
| | | | AAATGA | | | | |
| | | | (SEQ ID NO: 54) | | | | |
| | | IgG4 | GAGTCCAAATATCGTCCCCCGTGCCCATCATGCCCA | ESKYGPPGPSCPAPEFLGGPSVFLFPP | AJ001563 | | |
| | | variant | GCACCTGAGTTCCTGGGCCGACCATCAGTCTTCCTG | KPKDTLMISRTPEVTCVVVDVSQEDPE | | | |
| | | 2 | TTCCCCCCAAAACCCAAGGACACTCTCATGATCTCC | VQFNWYVDGVEVHNAKTKPREEQFNS | | | |
| | | | CGGACCCCTGAGGTCACGTGCGTGGTGGTGGACGTG | TRYVVSVLTVVHQDWLNGKEYKCKVS | | | |
| | | | AGCCAGGAAGACCCCGAGGTCCAGTTCAACTGGTAC | NKGLPSSIEKTISKAKGQPREPQVYTLP | | | |
| | | | GTGGATGCCGTGGAGGTGCATAATGCCAAGACAAAG | PSQEEMTKNQVSLTCLVKGFYPSDIAV | | | |
| | | | CCGCGGGAGGAGCAGTTCAACAGCACGTACCGTGTG | EWESNGQPENNYKTTPPVLDSDGSFFL | | | |
| | | | GTCAGCGTCCTCACCGTCGTGCACCAGGACTGGCTG | YSRLTVDKSRWQEGNVFSCSVMHEAL | | | |
| | | | AACGGCAAGGAGTACAAGTGCAAGGTCTCCAACAAA | NHNYTQKSLSLSLSGK* | | | |
| | | | GGCCTCCCGTCCTCCATCGAGAAAACCATCTCCAAA | (SEQ ID NO: 55) | | | |
| | | | GCCAAAGGGCAGCCCCGAGAGCCACAGGTGTACACC | | | | |
| | | | CTGCCCCCATCCCAGGAGGAGATGACCAAGAACCAG | | | | |
| | | | GTCAGCCTGACCTGCCTGGTCAAAGGCTTCTACCCC | | | | |
| | | | AGCGACATCGCCGTGGAGTGGGAGAGCAATGGGCAG | | | | |
| | | | CCGGAGAACAACTACAAGACCACGCCTCCCGTGCTG | | | | |
| | | | GACTCCGACGGCTCCTTCTTCCTCTACAGCAGGCTA | | | | |
| | | | ACCGTGGACAAGAGCAGGTGGCAGGAGGGGAATGTC | | | | |
| | | | TTCTCATGCTCCGTGATGCATGAGGCTCTGCACAAC | | | | |
| | | | CACTACACGCAGAAGAGCCTCTCCCTGTCTCTGGGT | | | | |
| | | | AAATGA | | | | |
| | | | (SEQ ID NO: 56) | | | | |
| | | IgG4 | GCACCTGAGTTCCTGGGGGGACCATCAGTCTTCCTG | APEFLGGPSVFLPPKPKDTLMISRTPE | AJ001584 | | |
| | | variant | TTCCCCCCAAAACCCAAGGACACTCTCATGATCTCC | VTCVVVDVSQEDPEVQFNWYVDGVEV | | | |
| | | 3 | CGGACCCCTGAGGTCACGTGCGTGGTGGTGGACGTG | HNAKTKPREEQFNSTYRVVSLVTVLHQ | | | |
| | | | AGCCAGGAAGACCCCGAGGTCCAGTTCAACTGGTAC | DWLNGKEYKCKVSNKGLPSSIEKTISKA | | | |
| | | | GTGGATGGCGTGGAGGTGCATAATGCCAAGACAAAG | KGQPREPQVYTLPPSQEEMTKNQVSL | | | |
| | | | CCGCGGGAGGAGCAGTTCAACAGCACGTACCGTGTG | TCLVKGFYPSDIAVEWESNGQPENNYK | | | |
| | | | GTCAGCGTCCTCACCGTCCTGCACCAGGACTGGCTG | TTPPVLDSDGSFFLYSKLTVDKSRWQE | | | |
| | | | AACGGCAAGGAGTACAAGTGCAAGGTCTCCAACAAA | GNVFSCSVMHEALHNYHTQKSLSLSLG | | | |
| | | | GGCCTCCCGTCCTCCATCGAGAAAACCATCTCCAAA | K* | | | |
| | | | GCCAAAGGGCAGCCCCGAGAGCCACAGGTGTACACC | (SEQ ID NO: 57) | | | |
| | | | CTGCCCCCATCCCAGGAGGAGATGACCAAGAACCAG | | | | |
| | | | GTCAGCCTGACCTGCCTGGTCAAAGGCTTCTACCCC | | | | |
| | | | AGCCACATCGCCGTCGAGTGGGAGAGCAATGGGCAG | | | | |
| | | | CCGGAGAACAACTACAAGACCACGCCTCCCGTGCTG | | | | |
| | | | GACTCCGACGGCTCCTTCTTCCTCTACAGCAAGCTC | | | | |
| | | | ACCGTGGACAAGAGCAGGTGGCAGGAGGGGAACGTC | | | | |
| | | | TTCTCATGCTCCGTGATGCATGAGGCTCTGCACAAC | | | | |
| | | | CACTACACGCAGAAGAGCCTCTCCCTGTCTCTGGGT | | | | |
| | | | AAATGA | | | | |
| | | | (SEQ ID NO: 58) | | | | |
| | Human Ig | Ig kappa | ACTGTGGCTGCACCATCTGTCTTCATCTTCCCGCCA | TVAAPSVFIFPPSDEQLKSGTASVVCL | X96754 | http://www. | None |
| | light chain | (CK) | TCTGATGAGCAGTTGAAATCTGGAACTGCCTCTGTT | LNNFYPREAKVQWKVDNALQSGNSQE | | IMGT | |
| | constant | | GTGTGCCTGCTGAATAACTTCTATCCCAGAGACGCC | SVTEQDSKDSTYSLSSTLTLSKADYEK | | repertoire | |
| | region | | AAAGTACAGTGGAAGGTGGATAACGCCCTCCAATCG | HKVYACEVTHQGLSSPVTKSFNRGEC* | | indes.php? | |
| | | | GGTAACTCCCAGGAGAGTGTCACAGAGCAGGACAGC | (SEQ ID NO: 59) | | section= | |
| | | | AAGGACAGCACCTACAGCCTCAGCAGCACCCTGACG | | | LocusGenes& | |
| | | | CTGAGCAAAGCAGACTACGAGAAACACAAAGTCTAC | | | repertoire= | |
| | | | GCCTGCGAAGTCACCCATCAGGGCCTGAGCTCGCCC | | | genetable& | |
| | | | GTCACAAAGAGCTTCAACAGGGGAGAGTGTTAG | | | species= | |
| | | | (SEQ ID NO: 60) | | | human&group= | |
| | | | | | | IGHC | |

The amino acid sequences as shown in SEQ ID NOS: 3, 21-28, 37, 39, 41, 43, 45, 47, 49, 51, 53, 55, 57 and 59 may have deletion(s), substitution(s) or addition(s) of one or several (e.g., up to five, about 10 at the most) amino acids. Even when such mutations are introduced, the resulting amino acid sequences may be capable of having the function as a constant region of Ig heavy chain or light chain.

Although the constant region of wild-type human IgG1 has ADCC activity and CDC activity, it is known that these activities can be reduced by introducing amino acid substitutions and deletions into specific sites. In the case of animals other than human where the constant region of an immunoglobulin equivalent to human IgG4 has not been identified, mutations may be introduced into the relevant region of an immunoglobulin equivalent to human IgG1 so that the resultant constant region with reduced ADCC activity and CDC activity can be used.

The present invention provides an artificial genetic DNA comprising (a′) a DNA encoding a light chain comprising a light chain variable region containing CDR1 having the amino acid sequence of QSLEYSDGYTY (SEQ ID NO: 16), CDR2 having the amino acid sequence of GVS and CDR3 having the amino acid sequence of FQATHDPDT (SEQ ID NO: 17) and the light chain constant region of an antibody of an animal other than rat and (b′) a DNA encoding a heavy chain comprising a heavy chain variable region containing CDR1 having the amino acid sequence of GFSLTSYY (SEQ ID NO: 18), CDR2 having the amino acid sequence of IRSGGST (SEQ ID NO: 19) and CDR3 having the amino acid sequence of ARTSSGYEGGFDY (SEQ ID NO: 20) and the heavy chain constant region of an antibody of an animal other than rat. The present invention also provides a DNA encoding a light chain comprising a light chain variable region containing CDR1 having the amino acid sequence of QSLEYSDGYTY (SEQ ID NO: 16), CDR2 having the amino acid sequence of GVS and CDR3 having the amino acid sequence of FQATHDPDT (SEQ ID NO: 17) and the light chain constant region of an antibody of an animal other than rat. Further, the present invention also provides a DNA encoding a heavy chain comprising a heavy chain variable region containing CDR1 having the amino acid sequence of GFSLTSYY (SEQ ID NO: 18), CDR2 having the amino acid sequence of IRSGGST (SEQ ID NO: 19) and CDR3 having the amino acid sequence of ARTSSGYEGGFDY (SEQ ID NO: 20) and the heavy chain constant region of an antibody of an animal other than rat.

For (a) a light chain comprising a light chain variable region containing CDR1 having the amino acid sequence of QSLEYSDGYTY (SEQ ID NO: 16), CDR2 having the amino acid sequence of GVS and CDR3 having the amino acid sequence of FQATHDPDT (SEQ ID NO: 17) and the light chain constant region of an antibody of an animal other than rat; and (b) a heavy chain comprising a heavy chain variable region containing CDR1 having the amino acid sequence of GFSLTSYY (SEQ ID NO: 18), CDR2 having the amino acid sequence of IRSGGST (SEQ ID NO: 19) and CDR3 having the amino acid sequence of ARTSSGYEGGFDY (SEQ ID NO: 20) and the heavy chain constant region of an antibody of an animal other than rat, reference should be had to the foregoing description. The DNA of (a′) is a DNA (gene) encoding the light chain of (a); and the DNA of (b′) is a DNA (gene) encoding the heavy chain of (b). An artificial genetic DNA comprising the DNA of (a′) and the DNA of (′b) may be synthesized on commercial synthesizer. Restriction enzyme recognition sites, KOZAK sequences, poly-A addition signal sequences, promoter sequences, intron sequences or the like may be added to the artificial genetic DNA.

The present invention also provides a vector comprising the above-mentioned artificial genetic DNA.

As the vector, Escherichia coli-derived plasmids (e.g., pBR322, pBR325, pUC12 or pUC13): Bacillus subtilis-derived plasmids (e.g., pUB110, pTP5 or pC194), yeast-derived plasmids e.g., pSH19 or pSH15); bacteriophages such as λ phage; animal viruses such as retrovirus or vaccinia virus; or insect pathogen viruses such as baculovirus may be used. In the Examples described later, pDN112 (Marti A, Yoshida R, Miyamoto H, Ishijima M, Suzuki Y, Higuchi M, Matsuyama Y, Igarashi M, Nakayama E, Kuroda M, Saijo M, Feldmann F, Brining D, Feldmann H, Takada A. PLoS One, 7: e36192, Apr. 27, 2012; Ikebuchi R, Konnai S, Okagawa T, Yokoyama K, Nakajima C, Suzuki Y, Murata S, Ohashi K. Immunology, 142(4): 551-561, Aug. 2014) was used.

The vector may also comprise promoters, enhancers, splicing signals, poly-A addition signals, intron sequences, selection markers, SV40 replication origins, and so forth.

The present invention also provides a host cell transformed by the above vector. It is possible to prepare the anti-PD-1 antibody of the invention by culturing the host cell and collecting the antibody of interest from the resultant culture. Therefore, the present invention also provides a method of preparing an antibody, comprising culturing the above-described host cell and collecting the anti-PD-1 antibody of the invention from the culture. In the method of the present invention for preparing an antibody, a vector incorporating an artificial genetic DNA comprising a DNA encoding the light chain and a DNA encoding the heavy chain may be transfected into a host cell. Alternatively, a vector incorporating a DNA encoding the light chain and a vector incorporating a DNA encoding the heavy chain may be co-transfected into a host cell.

Examples of the host cell include, but are not limited to, bacterial cells (such as Escherichia bacteria, Bacillus bacteria or Bacillus subtilis), fungal cells (such as yeast or Aspergillus), insect cells (such as S2 cells or Sf cells), animal cells (such as CHO cells, COS cells, HeLa cells, C127 cells, 3T3 cells, BHK cells or HEK 293 cells) and plant cells. Among these, CHO-DG44 cell (CHO-DG44(dfhr^{-/-})) which is a dihydrofolate reductase deficient cell is preferable.

Introduction of a recombinant vector into a host cell may be performed by the methods disclosed in Molecular Cloning 2nd Edition, J. Sambrook et al., Cold Spring Harbor Lab. Press, 1989 (e.g., the calcium phosphate method, the DEAE-dextran method, transfection, microinjection, lipofection, electroporation, transduction, scrape loading, the shotgun method, etc.) or by infection.

The resultant transformant may be cultured in a medium, followed by collection of the anti-PD-1 antibody of the present invention from the culture. When the antibody is secreted into the medium, the medium may be recovered, followed by isolation and purification of the antibody from the medium. When the antibody is produced within the transformed cells, the cells may be lysed, followed by isolation and purification of the antibody from the cell lysate.

Examples of the medium include, but are not limited to, OptiCHO medium, Dynamis medium, CD CHO medium, ActiCHO medium, FortiCHO medium, Ex-Cell CD CHO medium, BalanCD CHO medium, ProCHO 5 medium and Cellvento CHO-100 medium.

The pH of the medium varies depending on the cell to be cultured. Generally, a pH range from 6,8 to 7.6 is used; mostly, a pH range from 7.0 to 7.4 is appropriate.

When the cell to be cultured is CHO cells, culture may be performed by methods known to those skilled in the art. For example, it is usually possible to perform culturing in a gas-phase atmosphere having a CO₂ concentration of 0-40%, preferably 2-10%, at 30-39° C., preferably around 37° C.

The appropriate period of culture is usually from one day to three months, preferably from one day to three weeks.

Isolation and purification of the antibody may be performed by known methods. Known isolation/purification methods which may be used in the present invention include, but are not limited to, methods using difference in solubility (such as salting-out or solvent precipitation); methods using difference in molecular weight (such as dialysis, ultrafiltration, gel filtration or SDS-polyactylamide gel electrophoresis); methods using difference in electric charge (such as ion exchange chromatography); methods using specific affinity (such as affinity chromatography); methods using difference in hydrophobicity (such as reversed phase high performance liquid chromatography); and methods using difference in isoelectric point (such as isoelectric focusing).

The anti-PD-1 antibody of the present invention may be used as an antibody drug for animals or human. Therefore, the present invention provides a pharmaceutical composition comprising the above-described anti-PD-1 antibody as an active ingredient.

The pharmaceutical composition of the present invention may be used for prevention and/or treatment of cancers and/or infections. Examples of cancers and/or infections include, but are not limited to, neoplastic diseases (e.g., malignant melanoma, lung cancer, gastric cancer, renal cancer, breast cancer, bladder cancer, esophageal cancer, ovarian cancer and the like), leukemia, Johne's disease, anaplasmosis, bacterial mastitis, mycotic mastitis, mycoplasma infections (such as mycoplasma mastitis, mycoplasma pneumonia or the like), tuberculosis, Theileria orientalis infection, cryptosporidiosis, coccidiosis, trypanosomiasis and leishmaniasis.

The anti-PD-1 antibody of the present invention may be dissolved in buffers such as PBS, physiological saline or sterile water, optionally filter-sterilized with a filter or the like and then administered to animal subjects (including human) by injection. To the solution of this antibody, additives (such as coloring agents, emulsifiers, suspending agents, surfactants, solubilizers, stabilizers, preservatives, antioxidants, buffers, isotonizing agents, pH adjusters and the like) may be added. As routes of administration, intravenous, intramuscular, intraperitoneal, subcutaneous or intradermal administration and the like may be selected. Transnasal or oral administration may also be used.

The dose and the number of times and frequency of administration of the anti-PD-1 antibody of the present invention may vary depending on the symptoms, age and body weight of the animal subject, the method of administration, the dosage form and so on. For example, 0.1-100 mg/kg body weight, preferably 1-10 mg/kg body weight, per adult animal may usually be administered at least once at such a frequency that enables confirmation of desired effect.

While the pharmaceutical composition of the present invention may be used alone, it may be used in combination with surgical operations, radiation therapies, other immunotherapies such as cancer vaccine, or molecular target drugs. Synergistic effect can be expected from such combinations.

### EXAMPLES

Hereinbelow, the present invention will be described in more detail with reference to the following Examples. However, the present invention is not limited to these Examples.

### Example 1

### Establishment of Rat-Bovine Chimeric Anti-Bovine PD-1 Antibody Introduction

Programmed cell death 1 (PD-1), an immunoinhibitory receptor, and its ligand programmed cell death ligand 1 (PD-L1) are molecules identified by Prof, Tasuku Honjo et al., Kyoto University, as factors which inhibit excessive immune response and are deeply involved in immunotolerance. Recently, it has been elucidated that these molecules are also involved in immunosuppression in tumors. In the subject Example, for the purpose of establishing a novel therapy for bovine infections, a chimeric antibody gene was prepared in which variable region genes of rat anti-bovine PD-1 monoclonal antibody 5D2 capable of inhibiting the binding of bovine PD-1 to PD-L1 were linked to constant region genes of bovine immunoglobulins (bovine IgG1 and Igλ, with mutations having been introduced into the putative binding sites of Fcγ receptors in bovine IgG1's CH2 domain to inhibit ADCC activity; see FIGS. 1 and 11 for amino acid numbers and mutations: 250 E→P, 251 L→V, 252 P→A, 253 G→deletion, 347 A→S, 348 P→S; Ikebuchi R, Konnai S, Okagawa T, Yokoyama K, Nakajima C, Suzuki Y, Murata S. Ohashi K. Immunology, 142(4): 551-561, Aug. 2014). The resultant chimeric antibody gene was introduced into Chinese hamster ovary cells (DHO cells), which were cultured and proliferated to produce a rat-bovine chimeric anti-bovine PD-1antibody ch5D2. The effect of this chimeric antibody was confirmed in vitro and in vivo.

### Materials, Methods and Experimental Results

### 2.1. Construction of Bovine PD-1 and PD-L1 Expressing Cells

The nucleotide sequences of the full length cDNAs of bovine PD-1 gene (GenBank accession number AB510901; Ikebuchi R, Konnai 5, Sunden Y, Onuma M, Ohashi K. Microbiol. Immunol., 54(5): 291-298; May 2010) and bovine PD-Li gene (GenBank accession number AB510902; Ikebuchi R, Konnai S, Shirai T, Sunden Y, Murata S, Onuma M, Ohashi K. Vet. Res., 42: 103, Sep. 26. 2011) were determined. Based on the resultant genetic information, bovine PD-1 and bovine PD-L1 expressing cells were prepared. First, for preparing bovine PD-1 or PD-L1 expressing plasmid, PCR was performed using a synthesized bovine peripheral blood mononuclear cell (PBMC)-derived cDNA as a template and designed primers having NotI and HindIII (bovine PD-1) recognition sites or NheI and XhoI (bovine PD-L1) recognition sites on the 5′ side (boPD-1-myc F and R; or boPD-L1-EGFP F and R). The PCR products were digested with Nod (Takara) and HindIII (Takara; bovine PD-1) or NheI (Takara) and XhoI (Takara; bovine PD-L1), purified with FastGene Gel/PCR Extraction Kit (NIPPON Genetics) and cloned into pCMV-Tag1 vector (Agilent Technologies; bovine PD-1) or pEGFP-N2 vector (Clontech; bovine PD-L1) treated with the restriction enzymes in the same manner. The resultant expression plasmid of interest was extracted with QIAGEN Plasmid Midi kit (Qiagen) and stored at −30° C. until use in experiments. Hereinafter, the thus prepared expression plasmid is designated as pCMV-

Tag1-boPD-1 or pEGFP-N2-boPD-L1.
Primer (boPD-1-myc F):
(SEQ ID NO: 61)
ATATGCGGCCGCATGGGGACCCCGCGGGCGCT
Primer (boPD-1-myc R):
(SEQ ID NO: 62)
GCGCAAGCTTTCAGAGGGGCCAGGAGCAGT
Primer (boPD-L1-EGFP F):
(SEQ ID NO: 63)
CTAGCTAGCACCATGAGGATATATAGTGTCTTAAC
Primer (boPD-L1-EGFP R):
(SEQ ID NO: 64)
CAATCTCGAGTTACAGACAGAAGATGACTGC

Bovine PD-1 expressing cells were prepared by the procedures described below. First, 2.5 μg of pCMV-Tag1-boPD-1 was introduced into 4×10⁶ CHO-DG44 cells using Lipofectamine LTX (Invitrogen). Forty-eight hours later, the medium was exchanged with CD DG44 medium (Life Technologies) containing 800 μg/ml G418 (Enzo Life Science), 20 ml/L GlutaMAX supplement (Life Technologies), and 18 ml/L 10% Pluronic F-68 (Life Technologies), followed by selection. The resultant expression cells were reacted with rat anti-bovine PD-1 antibody 5D2 at room temperature. After washing, the cells were further reacted with anti-rat IgG microbead-labeled antibody (Miltenyi Biotec) at room temperature. Cells expressing bovine PD-1 at high levels were isolated with Auto MACS (Miltenyi Biotec). Subsequently, re-isolation was performed in the same manner to obtain still higher purity. The resultant expression cells were subjected to cloning by limiting dilution to thereby obtain a CHO DG44 cell clone expressing bovine PD-1 at high level (bovine PD-1 expressing cells).

Bovine PD-L1 membrane expressing cells were prepared by the procedures described below. First, 2.5 μg of pEGFP-N2-boPD-L1 or pEGFP-N2 (negative control) was introduced into 4×10⁶ CHO-DG44 cells using Lipofectamine LTX (Invitrogen). Forty-eight hours later, the medium was exchanged with CD DG44 medium (Life Technologies) containing 800 μg/ml G418 (Enzo Life Science), 20 ml/L GlutaMAX supplement (Life Technologies) and 18 ml/L 10% Pluronic F-68 (Life Technologies), followed by selection and cloning by limiting dilution (bovine PD-L1 expressing cell clone). In order to confirm the expression of bovine PD-L1 in the thus prepared cell clone, intracellular localization of EGFP was visualized with an inverted confocal laser microscope LSM700 (ZEISS).

### 2.2 Construction of Soluble Bovine PD-1

Bovine PD-1-Ig expressing plasmid was constructed by the procedures described below. Briefly, the signal peptide and the extracellular region of bovine PD-1 (GenBank accession number AB510901) were linked to the constant region of a known bovine IgG1 (GenBank accession number X62916) to prepare a gene sequence. After codons were optimized for CHO cells, gene synthesis was performed in such a manner that NotI recognition sequence, KOZAK sequence, bovine PD-1 signal peptide sequence, bovine PD-1 gene extracellular region sequence, bovine IgG1 Fc region sequence, and XbaI recognition sequence would be located in the gene in this order. It should be noted here that bovine IgG1 was mutated to inhibit ADCC activity; more specifically, mutations were introduced into the putative binding sites for Fcγ receptors of CH2 domain s of mutation: 185 E→P, 186 L→V, 187 P→A, 189 G→deletion, 281 A→S, 282 P→S; Ikebuchi R, Konnai S, Okagawa T, Yokoyama K, Nakajima C, Suzuki Y; Murata S, Ohashi K. Immunology, 142(4): 551-561, Aug. 204; the amino acid sequence of PD-1-Ig and the sites of mutation are disclosed in FIG. 2 of this article). The synthesized gene strand was digested with NotI (Takara) and XbaI (Takata), purified with FastGene Gel/PCR Extraction Kit (NIPPON Genetics), and incorporated into the cloning site (NotI and XbaI restriction enzyme recognition sequences downstream of PCMV and between INRBG and PABGH) of expression vector pDN11 (kindly provided by Prof. S. Suzuki, Hokkaido University Research Center for Zoonosis Control) treated with the restriction enzymes in the same manner, whereby bovine PD-1-Ig expressing vector was constructed. The expression plasmid was purified with QIAGEN Plasmid Midi kit (Qiagen) and stored at −30° C. until use in experiments. Hereinafter, the thus prepared expression plasmid is designated as pDN11-boPD-1-Ig.

Bovine PD-1-His expressing plasmid was prepared by the procedures described below. Briefly, for the purpose of amplifying the signal peptide and the extracellular region of bovine PD-1 (GenBank accession number AB510901), primers were designed in which NotI and XhoI recognition sites were added on the 5′ side (boPD-1-His F and R). A genetic sequence encoding a 6×His tag was added to the reverse primer. PCR was performed using a synthesized bovine PBMC-derived cDNA as a template. The respective PCR products were digested with NotI (Takara) and XhoI (Takara), purified with FastGene Gel/PCR Extraction Kit (NIPPON Genetics) and cloned into pCXN2.1(+) vector (Niwa H, Yamamura K, Miyazaki J. Gene, 108(2): 193-199; Dec. 15, 1991; kindly provided by Dr. T. Yokomizo, Juntendo University Graduate School of Medicine) treated with the restriction enzymes in the same manner. The resultant expression plasmid was purified with FastGene Xpress Plasmid PLUS Kit (NIPPON Genetics) and stored at −30° C. until use in experiments. Hereinafter, the thus prepared expression plasmid is designated as pCXN2.1-boPD-1-His.

Primer (boPD-1-His F):
(SEQ ID NO: 65)
ATAAGAATGCGGCCGCCACCATGGGGACCCCGCGGGCGCT
Primer (boPD-1-His R):
(SEQ ID NO: 66)
GCCCTCGAGTTATGGTGATGGTGATGGTGGATGACCAGGCTCTGCATCT

Soluble bovine PD-1-Ig expressing cells were prepared by the procedures described below. Briefly, 2.5 μg of pDN11-boPD-1-Ig was introduced into 4×10⁶ CHO-DG44 cells using Lipofectamine LTX (Invitrogen). Forty-eight hours later, the medium was exchanged with CD OptiCHO medium (Life Technologies) containing 800 μg/ml G418 (Enzo Life Science) and 20 ml/L GlutaMAX supplement (Life Technologies). After cultured for 3 weeks, the cells were subjected to selection. Briefly, the concentrations of the Fc fusion recombinant protein in the culture supernatants of the resultant cell clones were measured by ELISA using anti-bovine IgG F(c) rabbit polyclonal antibody (Rockland) to thereby select those cell clones that express the Fc fusion recombinant protein at high levels. The resultant highly expressing cell clone was transferred to a G418-free medium and cultured under shaking for 14 days, followed by collection of a culture supernatant. The culture supernatant containing the Fc fusion recombinants protein was ultrafiltered with Centricon Plus-70 (Millipore). Then, the Fc fusion recombinant protein was purified with Ab-Capcher Extra (ProteNova). After purification, the buffer was exchanged with phosphate-buffered physiological saline (PBS; pH 7.4) using PD-10 Desalting Column (GE Healthcare). The resultant protein was stored at −30° C. until use in experiments (bovine PD-1-Ig), The concentration of the purified bovine PD-1-1g was measured by ELISA using IgG F(c) rabbit polyclonal antibody (Rockland). For each washing operation in ELISA, Auto Plate Washer BIO WASHER 50 (DS Pharma Biomedical) was used. Absorbance was measured with Microplate Reader MTP-650FA (Corona Electric).

Soluble bovine PD-1-His expressing cells were prepared by the procedures described below. Briefly, 30 μg of pCXN2.1-boPD-1-His was introduced into 7.5×10⁷ Expi293F cells (Life Technologies) using Expifectamine (Life Technologies). After a 7-day culture under shaking, the culture supernatant was collected. The recombinant protein of interest was purified from the culture supernatant using TALON Metal Affinity Resin (Clontech; bovine PD-1-His). After purification, the buffer was exchanged with PBS (pH 7.4) using PD MiniTrap G-25 (GE Healthcare). The resultant protein was stored at −30° C. until use in experiments (bovine PD-1-His), The concentration of purified bovine PD-1-His was quantitatively determined in terms of the absorbance (280 nm) measured with Nanodrop8000 Spectrophotometer (Thermo Fisher Scientific).

### 2.3. Preparation of Rat Anti-Bovine PD-1 Monoclonal Antibody Producing Cells

Rat was immunized in the footpad with bovine PD-1-Ig (described above). Hybridomas were established by the iliac lymph node method to thereby obtain rat anti-bovine PD-1 monoclonal antibody producing hybridoma 5D2. With respect to the method of establishment of rat anti-bovine PD-1 monoclonal antibody, details are disclosed in the following non-patent document (Ikebuchi R, Konnai S, Okagawa I, Yokoyama K, Nakajima C, Suzuki Y, Murata S, Ohashi K. Vet. Res. 44: 59; Jul. 22, 2013).

### 2.4. Preparation of Rat-Bovine Chimeric Anti-Bovine PD-1 Antibody Expressing Vector

Rat-bovine chimeric anti-bovine PD-1 antibody ch5D2 was established by fusing the antibody constant regions of bovine IgG1 and Igλ, with rat anti-bovine PD-1 antibody 5D2 being used as antibody variable regions.

First, the genes of heavy chain and light chain variable regions were identified by the RACE method from a hybridoma that would produce rat anti-bovine PD-1 antibody 5D2. Subsequently, a gene sequence was prepared in which the heavy chain and the light chain variable regions of the rat anti-bovine PD-1 antibody 5D2 were linked to known constant regions of bovine IgG1 (heavy chain, modified from GenBank Accession number X62916) and bovine Ig2 (light chain; GenBank Accession number X62917), respectively. Then, codon optimization was carried out (SEQ ID NOS: 9 and 10 (amino acid sequences); SEQ ID NOS: 14 and 15 (nucleotide sequences after codon optimization)). It should be noted that bovine IgG1 had mutations added to the putative binding sites of Fcγ receptors in CH2 domain in order to suppress ADCC activity (See FIGS. 1 and 11 for amino acid numbers and mutations: 251 E→P, 252 L→V, 253 P→A, 254 G→deletion, 348 A→S, 349 P→S; Ikebuchi R, Konnai S, Okagawa T, Yokoyama K, Nakajima C, Suzuki Y, Murata S, Ohashi K. Immunology, 142(4): 551-561; Aug. 2014). Then, the gene was artificially synthesized in such a manner that NotI recognition sequence, KOZAK sequence, chimeric antibody light chain sequence, poly-A addition signal sequence (PABGH), promoter sequence (PCMV), SacI recognition sequence, intron sequence (INRBG), KOZAK sequence, chimeric antibody heavy chain sequence and XbaI recognition sequence would be located in this order. The synthesized gene strand was digested with NotI (Takara) and XbaI (Takara), purified with FastGene Gel/PCR Extraction Kit (NIPPON Genetics) and cloned into the cloning site (NotI and XbaI restriction enzyme recognition sequences downstream of PCMV and between INRBG and PABGH) of expression plasmid pDN112 (kindly provided by Prof. S. Suzuki, Hokkaido University Research Center for Zoonosis Control) treated with the restriction enzymes in the same manner (FIG. 2). The resultant plasmid of interest was extracted with QIAGEN Plasmid Midi kit (Qiagen) and stored at −30° C. until use in experiments. Hereinafter, the thus prepared expression plasmid is designated as pDN112-boPD-1ch5D2.

### 2.5. Expression of Rat-Bovine Chimeric Anti-Bovine PD-1 Antibody (FIG. 3)

The pDN112-boPD-1ch5D2 prepared above was transfected into CHO-DG44 cells (CHO-DG44(dfhr^{-/-})) which were a dihydrofolate reductase deficient cell. Forty-eight hours later, the medium was exchanged with CD OptiCHO medium (Life Technologies) containing 2 mM GlutaMAX supplement (Life Technologies) and 800 μg/ml G418 sulfate (Enzo Life Science). After cultured for 3 weeks, the expression cells were subjected to selection and cloning by limiting dilution. Subsequently, the concentrations of the chimeric antibody in the culture supernatants were measured by dot blotting and ELISA using anti-bovine IgG F(c) rabbit polyclonal antibody (Rockland) to thereby select high expression clones. Further, to the selected clones expressing rat-bovine chimeric anti-bovine PD-1 antibody at high levels were subjected to gene amplification treatment by adding a load with 60 nM methotrexate (Mtx; Wako)-containing medium. The thus established cell clone stably expressing rat-bovine chimeric anti-bovine PD-1 antibody was transferred into Mtx-free CD Opti-CHO medium and cultured wider shaking for 14 days (125 rpm, 37° C., 5% CO₂). Chimeric antibody production in the culture supernatant was measured by ELISA using anti-bovine IgG F(c) rabbit polyclonal antibody (Rockland). For each washing operation in ELISA, Auto Plate Washer BIO WASHER 50 (DS Pharma Biomedical) was used. Absorbance was measured with Microplate Reader MTP-650FA (Corona Electric). The culture supernatant at day 14 was centrifuged at 10,000 g for 10 min to remove cells, and the centrifugal supernatant was passed through a Steritop-GP 0.22 μm filter (Millipore) for sterilization and then stored at 4° C. until it was subjected to purification.

The results are shown in FIG. 3A. Among the rat-bovine chimeric anti-bovine PD-1 antibody expressing cell clones, the most productive clone secreted 91.7 mg/l of the chimeric antibody into the culture supernatant during the 14-day culture under shaking.

### 2.6. Purification of Rat-Bovine Chimeric Anti-Bovine PD-1 Antibody

From the culture supernatant prepared as described above, each chimeric antibody was purified using Ab Capcher Extra (ProteNova). An open column method was used for binding to resin; 1.5 M Glycine/3 M NaCl (pH 8.0) was used as equilibration buffer and wash buffer. As elution buffer, 0.1 M Glycine-HCl (pH 2.8) was used. As neutralization buffer, 1 M Tris (pH 9.0) was used. The purified antibody was subjected to buffer exchange with PBS (pH 7.41) using PD-10 Desalting Column (GE Healthcare) and concentrated using Amicon Ultra-1.5 (50 kDa, Millipore). The thus purified chimeric antibody was passed through a 0.22 μm syringe filter (Pall Life Sciences) for sterilization and stored at 4° C. until use in experiments.

### 2.7. Confirmation of the Purity of Purified Rat-Bovine Chimeric Anti-Bovine PD-1 Antibody (FIG. 3)

In order to confirm the purity of purified rat-bovine chimeric anti-bovine PD-1 antibody, antibody proteins were detected by SDS-PAGE and CBB staining. Purified rat-bovine chimeric anti-bovine PD-1 antibody ch5D2 was suspended in Laemmli Sample Buffer (Bio-Rad) and denatured at 95° C. for 5 min under reducing conditions (reduced with 2-mercaptoethaanol; Sigma-Aldrich) or under non-reducing conditions. The thus prepared samples were electrophoresed using 10% polyacrylamide gel. As molecular weight markers, Precision Plus Protein All Blue Standards (Bio-Rad) were used. After electrophoresis, the gel was stained with Quick-CBB kit (Wako) and subsequently decolored in distilled water.

The results are shown in FIG. 3B. Bands of rat-bovine chimeric anti-bovine PD-1 antibody were observed at predicted positions, that is, at 25 kDa (light chain) and 50 kDa (heavy chain) under reducing conditions and at 150 kDa under non-reducing conditions.

### 2.8. Binding Specificity of Rat-Bovine Chimeric Anti-Bovine PD-1 Antibody (FIG. 4)

It was confirmed by flow cytometry that rat-bovine chimeric anti-bovine PD-1 antibody specifically binds to bovine PD-1 expressing cells (described above). First, rat anti-bovine PD-1 antibody 5D2 or rat-bovine chimeric anti-bovine PD-1 antibody ch5D2 was reacted with bovine PD-1 expressing cells at room temperature for 30 min. After washing, Allophycocyanine (APC)-labeled anti-rat Ig goat antibody (Southern Biotech) or Alexa Fluor 647-labeled anti-bovine IgG (H+L) goat F(ab′)2 (Jackson ImmunoResearch) was reacted at room temperature for 30 min. As negative control antibody, rat IgG2a (κ) isotype control (BD Biosciences) or bovine IgG1 antibody (Bethyl) was used. After washing, each rat antibody or rat-bovine chimeric antibody bound to cell surfaces was detected by FACS Verse (BD Biosciences). For every washing operation and dilution of antibodies, PBS supplemented with 1% bovine serum albumin (Sigma-Aldrich) was used.

The experimental results are shown in FIG. 4. It was revealed that rat-bovine chimeric anti-bovine PD-1 antibody ch5D2 binds to bovine PD-1 expressing cells in the same manner as rat anti-bovine PD-1 antibody 5D2.

### 2.9. PD-1 Binding Avidity of Rat-Bovine Chimeric Anti-Bovine PD-1 Antibody

The binding avidities to bovine PD-1 of rat anti-bovine PD-1 antibody 5D2 and rat-bovine chimeric anti-bovine PD-1 antibody ch5D2 were measured by surface plasmon resonance using a bimolecular interaction analyzer (Biacore X100). Briefly, bovine PD-1-His (described above) was immobilized on a CM5 sensor chip (GE Healthcare) as a ligand. Subsequently, rat anti-bovine PD-1 antibody 5D2 or rat-bovine chimeric anti-bovine PD-1 antibody ch5D2 was reacted as an analyte, followed by single kinetics analysis. The experiment was repeated 3 times under the same conditions. Binding constant (kd value) and dissociation constant (ka value) were determined in each experiment, and binding avidity (KD value) was obtained.

The experimental results are shown in the table below. The binding avidity of rat-bovine chimeric anti-bovine PD-1 antibody for PD-1 protein was similar to that of rat anti-bovine PD-1 antibody 5D2, with no statistical difference observed (p>005; Welch's t-test).

| Anti- | | | |
|---|---|---|---|
| Bovine | | | |
| PD-1 | | | |
| Antibody | ka (1/Ms) | kd (1/s) | KD (M) |
| 5D2 | 1.84 × 10⁴ ± 0.27 | 2.15 × 10⁻⁴ ± 0.44 | 1.22 × 10⁻⁸ ± 0.39 |
| ch5D2 | 2.07 × 10⁴ ± 0.06 | 2.16 × 10⁻⁴ ± 1.12 | 1.05 × 10⁻⁸ ± 0.58 |

### 2.10. Blockade of Bovine PD-1/PD-L1 Binding by Rat-Bovine Chimeric Anti-Bovine PD-1 Antibody (FIG. 5)

Using bovine PD-L1 expressing cells (described above) and bovine PD-1-Ig (described above), bovine PD-1/PD-L1 binding inhibition by anti-PD-1 antibodies was tested. First, rat anti-bovine PD-1 antibody 5D2 or rat-bovine chimeric anti-bovine PD-1 antibody ch5D2 (final concentration: 0, 0,39, 0.78, 1.56, 3.12, 625, 12.5, 25 or 50 μg/ml) and bovine PD-1-Ig (final concentration: 5 μg/ml) labeled with biotin using Lightning-Link Type A Biotin Labeling Kit (Innova Biosciences) were added to 96-well plates, followed by reaction at 37° C. for 30 min. The resultant mixture was reacted with 1×10⁵ bovine PD-L1 expressing cells at 37° C. for 30 min. After washing, the reaction mixture was reacted with APC-labeled streptavidin (BioLegend) at room temperature for 30 min to thereby detect bovine PD-1-Ig bound to cell surfaces. For analysis. FACS Verse (BD Biosciences) was used. For every washing operation and dilution of antibodies, PBS supplemented with 1% bovine serum albumin (Sigma-Aldrich) was used. Taking the proportion of bovine PD-1-Ig-bound cells without addition of antibodies as 100%, the proportion of bovine PD-1-Ig-bound cells at each antibody concentration was shown as a relative value.

The experimental results are shown in FIG. 5. Rat-bovine chimeric anti-bovine PD-1 antibody ch5D2 inhibited the binding of PD-1-Ig to PD-L1 expressing cells by the same degree as rat anti-bovine PD-1 antibody 5D2 did.

### 2.11. CDR Analysis of Rat Anti-Bovine PD-1 Antibody

The complementarity-determining regions (CDRs) of rat anti-bovine PD-1 antibody 5D2 were determined using NCBI IGBLAST (http://www.ncbi.nlm.nih.gov/igblast/). The results are shown in FIG. 1.

### 2.12. Inoculation Test on Cattle with Rat-Bovine Chimeric Anti-Bovine PD-1 Antibody

Established rat-bovine chimeric anti-bovine PD-1 antibody ch5D2 (14 mg; 0.08 mg/kg) was intravenously administrated into an experimentally BIN-infected calf (Holstein, male, 4 months old, 173.5 kg). Blood samples were collected chronologically from the infected calf, followed by collection of blood (with heparin sodium (Ajinomoto) used as anticoagulant) and serum. Peripheral blood mononuclear cells (PBMCs) were isolated from the blood by density gradient centrifugation using Percoll (GE Healthcare).

### 2.13. Kinetics of Administered Rat-Bovine Chimeric Anti-Bovine PD-1 Antibody in Blood (FIG. 6)

Bovine PD-1-His (described above) was immobilized on ELISA plates (H type, Sumitomo Bakelite) at a final concentration of 10 μg/ml at 4° C. overnight. Subsequently, each well was washed with 200 μl of 0.05% Tween 20-supplemented Tris-buffered saline (TBS-T) five times, followed by blocking with 1% skim milk-supplemented TBS-T at room temperature for 1 hr. Another washing was carried out in the same manner. The serum collected from the test calf was added to each well and reacted at room temperature for 1 hr. After washing, horseradish peroxidase-labeled anti-bovine IgG F(c) rabbit polyclonal antibody (Rockland) was reacted at room temperature for 1 hr. Each well was washed again and then TMB One Component Substrate (Bethyl) was added for coloring. The enzyme reaction was terminated with 0.18 M dilute sulfuric acid. Absorbance (450 nm) was measured with Microplate Reader MTP-650FA (Corona Electric). For every plate washing operation, Auto Plate Washer BIO WASHER 50 (DS Pharma. Biomedical) was used.

The experimental results are shown in FIG. 6. Rat-bovine chimeric anti-bovine PD-1 antibody was detected in the serum of the test calf until 70 days after administration (at the end of the clinical test). The antibody retained particularly high concentrations for one week after administration.

### 2.14. Cell Proliferation Response of T Cells to BIN Antigen (FIG. 7)

Bovine PBMCs were suspended in PBS and reacted with carboxyfluorescein succinimidyl ester (CFSE; Invitrogen) at room temperature for 20 min for labeling. After washing twice with RPMI 1640 medium (Sigma-Aldrich) containing 10% inactivated fetal bovine serum (Cell Culture Technologies), penicillin 200 U/ml, streptomycin 200 μg/ml and 0.01% L-glutamine (Life Technologies), cell concentration was adjusted to 4×10⁵ cells/ml using the same medium. To the PBMCs, culture supernatant of 2% BLV-infected fetal lamb kidney cell (FLK-BLV), culture supernatant of fetal lamb kidney cell (FLK) not infected with 2% BLV, or BLV gp51 peptide mix 0.1 μg/ml or 1 μg/ml was added, followed by a 6-day culture at 37° C. under 5% CO₂. After 6 days, PBMCs were recovered and reacted with Alexa Fluor 647-labeled mouse anti-bovine CD4 antibody (CC30, AbD Serotec). Peridinin-chlorophyll-protein complex/cyanin 5.5-labeled mouse anti-bovine CD8 antibody (CC63, AbD Serotec) and R-Phycoerythrin/cyanin 7 (PE/Cy7)-labeled anti-bovine IgM mouse antibody (IL-A30, AbD Serotec) at 4° C. for 20 min. For labeling antibodies, Zenon Mouse IgG1 Labeling Kits (Life Technologies) or Lightning-Link Kits (Innova Biosciences) was used. For analysis, FACS Verse (BD Biosciences) was used. For every washing operation and dilution of antibodies, PBS supplemented with 1% bovine serum albumin (Sigma-Aldrich) was used. With respect to the proportion of proliferated T cells (CFSE^{low} cells), statistical test was performed using the method of Dunnett.

The experimental results are shown in FIG. 7. Upon administration of rat-bovine chimeric anti-bovine PD-1 antibody, BLV-specific cell proliferative response in CD4⁺ T cells showed a statistically significant increase immediately after the administration, compared to the response before administration.

### 2.15. Transition in BLV Proviral Load (FIG. 8)

DNA was extracted from isolated bovine PBMCs using Wizard DNA Purification kit (Promega). The concentration of the extracted DNA was quantitatively determined based on the absorbance (260 nm) measured with Nanodrop 8000 Spectrophotometer (Thermo Fisher Scientific). For measuring BIN proviral load in PBMCs, real time PCR was performed using Cycleave PCR Reaction Mix SP (Takara) and Probe/Primer/Positive control (Takara) for bovine leukemia virus detection. LightCycler480 System II (Roche Diagnosis) was used for the measurement. With respect to the measured proviral load, statistical test was performed by the method of Dunnett.

The experimental results are shown in FIG. 8. Upon administration of rat-bovine chimeric anti-bovine PD-1 antibody, BLV proviral load in PBMCs showed a statistically significant decrease immediately after the administration, compared to the load before administration. The BLV proviral load remained at low levels until the end of the clinical test (day 70).

### Example 2

### Application of Anti-PD-1 Antibody to Other Animal Species

### 1. Materials, Methods and Experimental Results

### 1.1. Identification of Ovine and Water Buffalo PD-1 Genes

In order to determine the full-lengths of the coding sequences (CDSs) of ovine and water buffalo PD-1 cDNAs, primers for amplifying the full lengths of CDSs were first designed (ovPD-1 CDS F and R; buPD-1 CDS F1, R1, F2 and R2) based on the nucleotide sequences of ovine and water buffalo PD-1 genes (GenBank accession numbers BC123854 and XM 012176227), and then PCR was performed using a synthesized ovine or water buffalo PBMC-derived cDNA as a template. For the resultant amplified products, nucleotide sequences were determined with a capillary sequencer according to conventional methods (Mingala C N, Konnai S, Ikebuchi R, Ohashi K. Comp. Immunol. Microbiol. Infect. Dis., 34(1): 55-63; Jan. 2011; Water buffalo PD-1 gene was identified in this article).

Primer (ovPD-1 CDS F):
(SEQ ID NO: 67)
ATGGGGACCCCGCGGGCGCC
Primer (ovPD-1 CDS R):
(SEQ ID NO: 68)
TCAGAGGGGCCAGGAGCAGTGTCCA
Primer (buPD-1 CDS F1):
(SEQ ID NO: 69)
ATGGGGACCCCGCGGGCGCT
Primer (buPD-1 CDS R1):
(SEQ ID NO: 70)
GATGACCAGGCTCTGCATCT
Primer (buPD-1 CDS F2):
(SEQ ID NO: 71)
AATGACAGCGGCGTCTACTT
Primer (buPD-1 CDS R2):
(SEQ ID NO: 72)
TCAGAGGGGCCAGGAGCAGT

### 1.2. Construction of Ovine PD-1 Expressing COS-7 Cells

In order to prepare ovine PD-1 expression plasmid, PCR was performed using a synthesized ovine PBMC-derived cDNA as a template and primers designed by adding BglII and SmaI recognition sites on the 5′ side (ovPD-1-EGFP F and R). The resultant PCR products were digested with BglII (Takara) and SmaI (Takara), purified with FastGene Gel/PCR. Extraction Kit (NIPPON Genetics) and cloned into pEGFP-N2 vector (Clontech) treated with the restriction enzymes in the same manner. The expression plasmid of interest was extracted using FastGene Xpress Plasmid PLUS Kit (NIPPON Genetics) and stored at −30° C., until use in experiments. Hereinafter, the thus prepared plasmid is designated as pEGFP-N2-ovPD-1.

Primer (ovPD-1-EGFP F):
(SEQ ID NO: 73)
GAAGATCTATGGGGACCCCGCGGGCGCCG
Primer (ovPD-1-EGFP R):
(SEQ ID NO: 74)
GACCCGGGGAGGGGCCAGGAGCAGTGTCC

COS-7 cells were subcultured at a density of 5×10⁴ cells/cm² in 6-well plates, and then cultured overnight in RPMI 1640 medium containing 10% inactivated fetal bovine serum (Invitrogen) and 0.01% L-glutamine (Life Technologies) at 37° C. in the presence of 5% CO₂. The pEGFP-N2-ovPD-1 or pEGFP-N2 (negative control) was introduced into COS-7 cells at 0.4 μg/cm² using Lipofectamine 2000 (Invitrogen). The cells were cultured for 48 hours (ovPD-1-EGFP expressing cells). In order to confirm the expression of ovine PD-1 in the thus prepared expressing cells, intracellular localization of EGFP was visualized with an all-in-one fluorescence microscope BZ-9000 (KEYENCE).

### 1.3. Reactivity of Rat Anti-Bovine PD-1 Antibody 5D2 with Ovine PD-1 (FIG. 9)

It was confirmed by flow cytometry that rat anti-bovine PD-1 monoclonal antibody cross-reacts with ovine PD-1. Ovine PD-1 -EGFP expressing COS-7 cells were blocked with 10% inactivated goat serum (Invitrogen)-supplemented PBS at room temperature for 15 min and reacted with 10 μg/ml of rat anti-bovine PD-1 antibody 5D2 at room temperature for 30 min. After washing, the cells were reacted with APC-labeled anti-rat Ig goat antibody (Beckman Coulter) at room temperature for 30 min. As a negative control antibody, rat IgG2a (κ) isotype control (BD Bioscience) was used. For analysis, FACS Verse (BD Bioscience) was used. For every washing operation and dilution of antibodies, 1% bovine serum albumin (Sigma-Aldrich)-supplemented PBS was used.

The experimental results are shown in FIG. 9. It was confirmed that rat anti-bovine PD-1 antibody 5D2 binds to ovine PD-1 expressing cells.

### 1.4. Reactivity of Rat Anti-Bovine PD-1 Antibody 5D2 with Water Buffalo Lymphocytes (FIG. 10)

Peripheral blood mononuclear cells (PBMCs) were isolated from peripheral blood of water buffalo (Bubalus ubalis; Asian water buffalo) by density gradient centrifugation using Percoll (GE Healthcare). The isolated water buffalo PBMCs were suspended in RPMI 1640 medium (Sigma-Aldrich) containing 10% inactivated fetal bovine serum (Cell Culture Technologies), penicillin 200 U/ml, streptomycin 200 μg/ml and 0.01% L-glutamine (Life Technologies). Cell density was adjusted to 2×10⁶ cells/ml. To these PBMCs, phorbol 12-myristate acetate (PMA) 20 ng/ml and ionomvcin 1 μg/ml (Sigma-Aldrich) were added, followed by a 2-day culture at 37° C. under 5% CO₂. Cultured PBMCs were harvested and blocked with 10% inactivated goat serum (Invitrogen)-supplemented. PBS at room temperature for 15 min. Then, rat anti-bovine PD-1 antibody 5D2 and mouse anti-bovine CD8 antibody (38.65, AbD Serotec) were reacted at room temperature for 30 min. As a negative control, rat IgG2a (κ) isotype control (BD Bioscience) was used. After washing, APC-labeled goat anti-rat Ig antibody (Beckman Coulter) and PE-labeled goat anti mouse IgG antibody (Beckman Coulter) were reacted at room temperature for 30 min. After further washing. Alexa Flour488-labeled mouse anti-bovine CD4 antibody (CC30, AbD Serotec) and PE/Cy7-labeled anti-bovine IgM mouse antibody (IL-A30, AbD Serotec) were reacted at room temperature for 30 min. For antibody labeling, Zenon Mouse IgG1 Labeling Kits (Life Technologies) or Lightning-Link Kits (Innova Biosciences) was used. For analysis, FACS Verse (BD Biosciences) was used. For every washing operation and dilution of antibodies, 10% inactivated goat serum (Invitrogen)-supplemented PBS was used.

The experimental results are shown in FIG. 10. Rat anti-bovine PD-1 antibody 5D2 strongly bound to water buffalo CD4⁺ T cells (IgM⁻CD4⁺) and CD8⁺ T cells (IgM⁻CD8⁺) that had been activated by PMA/ionomycin stimulation.

### Example 3

### Binding to Bovine Fcγ Receptors of Rat-Bovine Chimeric Anti-Bovine PD-1 Antibody Having Wild-Type or Mutated Bovine IgG1

### Introduction

The present inventors have established a rat-bovine chimeric anti-bovine PD-1 antibody in Example 1 with a view to establishing a novel therapy for bovine infections. In the process, mutations were added to putative binding sites for Fcγ receptors in bovine IgG1 CH2 domain in order to suppress ADCC activity mediated by the chimeric antibody (FIGS. 1 and 11). In the subject Example, in order to examine the effect of these amino acid mutations, the present inventors prepared rat-bovine chimeric anti-bovine PD-1 antibodies having mutated bovine IgG1 (“IgG1 ADCC-” described above) and wild-type bovine IgG1 (“IgG1 WT”), respectively, and confirmed their binding to known bovine Fcγ receptors.

### Materials, Methods and Experimental Results

### 2.1. Preparation of Rat-Bovine Chimeric Anti-Bovine PD-1 Antibody Expressing Vector

Rat-bovine chimeric anti-bovine PD-1 antibody ch5D2 having wild-type bovine IgG1 (IgG1 WT) or mutated bovine IgG1 (IgG1 ADCC- described above) was established.

An expression plasmid encoding rat-bovine chimeric anti-bovine PD-1 antibody ch5D2 having mutated bovine IgG1 (IgG1 ADCC-) was prepared according to the procedures described in Example 1 (SEQ ID NOS: 9 and 10 (amino acid sequences), SEQ II) NOS: 14 and 15 (nucleotide sequences after codon optimization)). It should be noted that in order to suppress ADCC activity, the bovine IgG1 used in ch5D2 IgG1 ADCC- had mutations added to the putative binding sites for Fcγ receptors in CH2 domain (see FIGS. 1 and 11 for amino acid numbers and mutations: 251 E→P, 252 L→V, 253 P→A, 254 G→deletion, 348 A→S, 349 P→S; Ikebuchi R, Konnai S, Okagawa I, Yokoyama K, Nakajima C, Suzuki Y, Murata S, Ohashi K. Immunology, 142(4): 551-561; Aug. 2014). Hereinafter, the thus prepared plasmid is designated as pDN112-boPD-1 ch5D2 IgG1 ADCC-.

An expression plasmid encoding rat-bovine chimeric anti-bovine PD-1 antibody ch5D2 having wild-type IgG1 (IgG1 WT) was prepared according to the procedures described below. First, in order to amplify the gene encoding the constant region of wild-type bovine IgG1. (GenBank accession number X62916), PCR was performed using a synthesized bovine PBMC-derived cDNA as a template and designed primers that have NheI and XbaI recognition sites added on the 5′ side (boIgG1 CH1 F and boIgG1 CH3 R). The amplified gene strand was digested with NheI (Takara) and XbaI (Takara), purified with FastGene Gel/PCR Extraction Kit (NIPPON Genetics), and cloned into pDN112-boPD-1ch5D2 IgG1 ADCC- that had been treated with the restriction enzymes in the same manner. Further, the resultant plasmid was purified with QIAGEN Plasmid Midi kit (Qiagen) and digested with NotI (Takara) and XbaI (Takara) to thereby obtain an expression cassette for ch5D2's light chain (SEQ ID NO: 9 (amino acid sequence), SEQ ID NO: 14 (nucleotide sequence)) and heavy chain (IgG1 WT) (SEQ ID NO: 75 (amino acid sequence), SEQ ID NO: 76 (nucleotide sequence)). This gene fragment was purified with FastGene Gel/PCR Extraction Kit (NIPPON Genetics) and cloned into the cloning site (NotI and XbaI restriction enzyme recognition sequences downstream of PCMV and between INRBG and PABGH) of expression vector pDC6 (kindly provided by Prof. S. Suzuki, Hokkaido University Research Center for Zoonosis Control) (FIG. 12). The resultant expression plasmid of interest was extracted with QIAGEN Plasmid Midi kit (Qiagen) and stored at 30° C. until use in experiments. Hereinafter, the thus prepared expression plasmid is designated as pDC6-boPD-1ch5D2 IgG1 WT.

Primer (boIgG1 CH1 F):
(SEQ ID NO: 77)
CTAGCTAGCACCACAGCCCCGAAAGTCT
Primer (boIgG1 CH3 R):
(SEQ ID NO: 78)
TGCTCTAGATTATTTACCCGCAGACCTTAGA

### 2.2. Expression and Purification of Rat-Bovine Chimeric Anti-Bovine PD-1 Antibody

Thirty micrograms of pDC6-boPD-1ch5D2 IgG1 WT or pDN112-boPD-1ch5D2 IgG1 ADCC- was introduced into 7.5×10⁷ Expi293F cells (Life Technologies) using Expifectamine (Life Technologies) and the transfected cells were then cultured under shaking for 5 to 7 days, followed by collection of a culture supernatant. Each chimeric antibody was purified from the culture supernatant using Ab Capcher Extra (ProteNova). An open column method was used for binding to resin; 1.5 M Glycine/3 M NaCl (pH 8.0) was used as equilibration buffer and wash buffer. As elution buffer, 0.1 M Glycine-HCl (pH 2.8) was used. As neutralization buffer, 1M Tris (pH 9.0) was used. The purified antibody was subjected to buffer exchange with PBS (pH 7.4) using PD-10 Desalting Column (GE Healthcare) and concentrated using Amicon Ultra-15 (50 kDa, Millipore). The thus purified chimeric antibody was passed through a 0.22 μm syringe filter (Pall Life Science) for sterilization and stored at 4° C. until use in experiments. The concentration of each chimeric antibody as purified was quantitatively determined with the absorbance (280 nm) measured with Nanodrop8000 Spectrophotometer (Thermo Fisher Scientific).

### 2.3. Confirmation of the Purity of Purified Rat-Bovine Chimeric Anti-Bovine PD-1 Antibodies (FIG. 13)

In order to confirm the purity of purified rat-bovine chimeric anti-bovine PD-1 antibodies (ch5D2 IgG1 WT and ch5D2 IgG1 ADCC-), antibody proteins were detected by SDS-PAGE and CBB staining. Each chimeric antibody purified was suspended in Laemmli Sample Buffer (Bio-Rad) and denatured at 9.5° C. for 5 min under reducing conditions (reduced with 2-mercaptoethaanol; Sigma-Aldrich) or under non-reducing conditions. The thus prepared samples were electrophoresed using SuperSep Ace 5%-20% gradient polyacrylamide gel (Wako). As molecular weight markers, Precision Plus Protein All Blue Standards (Bio-Rad) were used. After electrophoresis, the gel was stained with Quick-CBB kit (Wako) and decolored in distilled water.

The results are shown in FIG. 13. Bands of ch5D2IgG1 WT and ch5D2 IgG1 ADCC- were observed at predicted positions, that is, at 25 kDa (light chain) and 50 kDa (heavy chain) under reducing conditions and at 150 kDa under non-reducing conditions.

### 2.4. Construction of Soluble Bovine Fcγ Receptors (FcγRs)

Bovine FcγRI-His, FcγRIII-His and Fcγ2R-His expressing plasmids were constructed according to the procedures described below, in order to amplify the signal peptide and the extracellular region of bovine FcγRI, FcγRII, FcγRIII and Fcγ2R (GenBank accession numbers NM_174538, NM_174539, NM_001077402 and NM_001001138), primers were designed which had NotI and XhoI recognition sites added on the 5′ side (boFcγRI-His F and R; boFcγRIII-His F and R; or bocγ2R-His F and R) or NheI and EcoRV recognition sites added on the 5′ side (boFcγRIII-His F and R). A gene sequence encoding a 6×His tag was added to reverse primers. PCR was performed using a synthesized bovine PBMC-derived cDNA as a template. The respective PCR products were digested with NotI (Takara) and XhoI (Takara) (FcγRI-His, FcγRIII-His and Fcγ2R-His) or NheI (Takara) and EcoRV (Takara) (FcγRII-His), purified with FastGene Gel/PCR Extraction Kit (NIPPON Genetics) and cloned into pCXN2.1(+) vector (Nivea H. Yamamura K, Miyazaki. J. Gene, 108(2): 193-199; Dec. 15, 1991; kindly provided by Dr. T. Yokomizo, Juntendo University Graduate School of Medicine). The resultant expression plasmids were purified with FastGene Xpress Plasmid PLUS Kit (NIPPON Genetics) and stored at −30° C. until use in experiments. Hereinafter, the thus prepared expression plasmid is designated as pCXN2.1-boFcγRI-His, pCXN2.1-boFcγRII-His, pCXN2.1-boFcγRIII-His or pCXN2.1-boFcγ2R-His.

Primer (boFcγRI-His F):
(SEQ ID NO: 79)
ATAAGAATGCGGCCGCCACCATGTGGCTCATAATAGCTCT
Primer (boFcγRI-His R):
(SEQ ID NO: 80)
GCCCTCGAGTTAATGGTGATGGTGATGGTGAGGAGTTGTTGACTGGAGGC
Primer (boFcγRII-His F):
(SEQ ID NO: 81)
ATAAGAATGCTAGCCACCATGGGGATCCCCTCATTCCT
Primer (boFcγRII-His R):
(SEQ ID NO: 82)
GCCGATATCTTAATGGTGATGGTGATGGTGCGATGAGGGGCCGCTCGAGC
Primer (boFcγRIII-His F):
(SEQ ID NO: 83)
ATAAGAATGCGGCCGCCACCATGTGGCAACTGCTACCACC
Primer (boFcγRIII-His R):
(SEQ ID NO: 84)
GCCCTCGAGTTAATGGTGATGGTGATGGTGGTGCCAAGGTAGAAAGAATG
Primer (boFcγ2R-His F):
(SEQ ID NO: 85)
ATAAGAATGCGGCCGCCACCATGGCCCCCACCCTCCCTGCCTTGCTCT
Primer (boFcγ2R-His R):
(SEQ ID NO: 86)
GCCCTCGAGTTAATGGTGATGGTGATGGTGATTCTGCATCGTGTAGTCTG

Soluble bovine FcγRI-His, FcγRII-His, FcγRIII-His and Fcγ2R-His expressing cells were prepared according to the procedures described below Briefly, 30 μg of pCXN2.1-boFcγRI-His, pCXN2.1-boFcγRII-His, pCXN2.1-boFcγRIII-His or pCXN2.1-boFcγ2R-His was introduced into 7.5×10⁷ Expi293F cells (Life Technologies) using Expifectamine (Life Technologies) and the transfected cells were then cultured under shaking for 5 to 7 days, followed by collection of a culture supernatant. Recombinant proteins were purified from the culture supernatant using TALON Metal Affinity Resin (Clontech). After purification, the buffer was exchanged with PBS (pH 7.4) using Amicon Ultra-15 Centrifugal Filter Unit (10 kDa, Millipore), and the recombinant proteins were stored at −30° C. until use in experiments (bovine PD-1-His). The concentrations of purified bovine FcγRI-His, FcγRII-His, FcγRIII-His and Fcγ2R-His were quantitatively determined in terms of the absorbance (280 nm) measured with Nanodrop8000 Spectrophotometer (Thermo Fisher Scientific).

### 2.5. Binding to Bovine FcγRs of Rat-Bovine Chimeric Anti-Bovine PD-1 Antibody ch5D2 IgG1 WT and IgG1 ADCC- (FIG. 14)

Rat-bovine chimeric anti-bovine PD-1 antibody ch5D2 IgG1 WT or IgG1 ADCC- was immobilized on Nunc MaxiSorp ELISA plates (Nunc) at a final concentration of 50, 25, 12.5, 6.25, 3.12 or 1.5610 nM at 37° C. for 2 hr. Subsequently, each well was washed with 200 ᵢd of 0.05% Tween 20-supplemented PBS (PBS-T) five times, followed by blocking with SuperBlock (PBS) Blocking Buffer (Thermo Fisher Scientific) at 37° C. for 30 min, Each well was washed again in the same manner. Then, bovine FcγRI-His, FcγRII-His, FcγRIII-His or Fcγ2R-His was added to each well at a final concentration of 10 μg/ml and reacted at 37° C. for 1 hr. After washing, anti-polyhistidine tag mouse monoclonal antibody (Abeam) was reacted at 37° C. for 30 min. Subsequently, each well was washed, and horseradish peroxidase-labeled anti-mouse IgG goat polyclonal antibody (MP Biomedicals) was reacted at 37° C. for 30 min. Each well was washed again, and then TMB One Component Substrate (Bethyl) was added for coloring. Thereafter, the enzyme reaction was terminated with 0.18 M dilute sulfuric acid, and absorbance (450 nm) was measured with Microplate Reader MTP-900 (Corona Electric), For every plate washing operation, Auto Plate Washer BIO WASHER 50 (DS Pharma Biomedical) was used.

The experimental results are shown in FIG. 14. IgG1 WT strongly bound to bovine FcγRI-His and weakly bound to bovine FcγRII-His. On the other hand, IgG1 ADCC- did not bind to bovine FcγRI-His or FcγRII-His. Neither IgG1 WT nor IgG1 ADCC- bound to bovine FcγRIII-His or Fcγ2R-His.

All publications, patents and patent applications cited herein are incorporated herein by reference in their entirety.

### INDUSTRIAL APPLICABILITY

The anti-PD-1 antibody of the present invention is applicable to prevention and/or treatment of cancers and infections of animals.

SEQUENCE LISTING FREE TEXT

<SEQ ID NO: 1>
SEQ ID NO: 1 shows the amino acid sequence of the light chain variable region (VL) of a rat
anti-bovine PD-1 antibody, Underlined parts: CDR1, CDR2 and CDR3 in this order from the
NH2 terminus.
MKVPGRLLVLLFWIPASRSDVVLTQTPVSLSVFLGDQASISCRSSQSLEYSDGYTYLE
WYLQKPGQSPQLLIYGVSNRFSGVPDRFIGSGSGTDFTLKISRVEPEDLGVYYCFQAT
HDPDTFGAGTKLELK
<SEQ ID NO: 2>
SEQ ID NO: 2 shows the amino acid sequence of the heavy chain variable region (VH) of a
rat anti-bovine PD-1 antibody, Underlined parts: CDR1, CDR2 and CDR3 in this order from
the NH2 terminus.
MAILVLLLCLVTIPHSVLSQVQLKETGPGLVQPTQTLSITCTVSGFSLTSYYIQWVRQT
PGKGLEWMGFIRSGGSTEYNSEFKSRLSINRDTSKNQVFLKMNSLKTEDTGVYYCAR
TSSGYEGGFDYWGQGVMVTVSS
<SEQ ID NO: 3>
SEQ ID NO: 3 shows the amino acid sequence of the light chain constant region (CL) of a
bovine antibody (bovine Ig lambda, GenBank: X62917),
QPKSPPSVTLFPPSTEELNGNKATLVCLISDFYPGSVTVVWKADGSTITRNVETTRASK
QSNSKYAASSYLSLTSSDWKSKGSYSCEVTHEGSTVTKTVKPSECS
<SEQ ID NO: 4>
SEQ ID NO: 4 shows the amino acid sequence of the heavy chain constant region (CH) of a
bovine antibody (bovine IgG1, modified from Gen:Bank: X62916). Mutated parts are
underlined. Amino acid numbers and mutations: 123 E→P, 124 L→V, 125 P→A, 126 G→
deletion, 218 A→S, 219 P→S
ASTTAPKVYPLSSCCGDKSSSTVTLGCLVSSYMPEPVTVTWNSGALKSGVHTFPAVL
QSSGLYSLSSMVTVPGSTSGQTFTCNVAHPASSTKVDKAVDPTCKPSPCDCCPPPPVA
GPSVFIFPPKPKDTLTISGTPEVTCVVVDVGHDDPEVKFSWFVDDVEVNTATTKPREE
QFNSTYRVVSALRIQHQDWTGGKEFKCKVHNEGLPSSIVRTISRTKGPAREPQVYVLA
PPQEELSKSTVSLTCMVTSFYPDYIAVEWQRNGQPESEDKYGTTPPQLDADSSYFLYS
KLRVDRNSWQEGDTYTCVVMHEALHNHYTQKSTSKSAGK
<SEQ ID NO: 5>
SEQ ID NO: 5 shows the nucleotide sequence of the VL of a rat anti-bovine PD-1 antibody.
ATGAAAGTGCCTGGTAGGCTGCTGGTGCTGTTGTTTTGGATTCCAGCTTCCAGGAG
TGATGTTGTGTTGACACAAACTCCAGTTTCCCTGTCTGTCACACTTGGAGATCAAG
CTTCTATATCTTGCAGGTCTAGTCAGAGCCTGGAATATAGTGATGGATACACTTATT
TGGAATGGTACCTACAGAAGCCAGGCCAGTCTCCACAGCTCCTCATCTATGGAGTT
TCCAACCGATTTTCTGGGGTCCCAGACAGGTTCATTGGCAGTGGGTCAGGGACAG
ATTTCACCCTCAAGATCAGCAGAGTAGAGCCTGAGGACTTGGGAGTTTATTACTGC
TTCCAAGCTACACATGATCCGGACACGTTTGGAGCTGGGACCAAGCTGGAACTGA
AA
The nucleotide sequence of SEQ ID NO: 5 after codon optimization is shown
in <SEQ ID NO: 11>.
ATGAAGGTCCCTGGTAGGCTGCTGGTTCTCTTGTTCTGGATCCCTGCTTCCAGAAG
TGACGTGGTGCTGACTCAAACACCAGTGAGTCTCAGTGTGACCCTCGGCGACCA
GGCCTCCATTTCTTGCCGTAGCAGCCAGTCCTTGGAATACTCTGATGGTTATACTTA
TCTGGAGTGGTACCTCCAGAAGCCCGGGCAGTCACCCCAGCTTCTTATCTACGGT
GTGAGCAACAGATTTTCTGGGGTTCCTGATCGGTTTATTGGATCTGGATCCGGTAC
CGACTTCACATTGAAAATTTCACGCGTCGAACCCGAGGATTTGGGGGTCTACTATT
GCTTCCAAGCCACCCACGATCCCGACACCTTCGGCGCTGGCACTAAGCTGGAGCT
GAAA
<SEQ ID NO: 6>
SEQ ID NO: 6 shows the nucleotide sequence of the VH of a rat anti-bovine PD-1 antibody.
ATGGCTATCCTGGTGCTGCTTCTCTGCCTGGTGACCATTCCACACTCTGTCTTGTCC
CAGGTGCAGCTGAAGGAGACAGGACCTGGCCTGGTGCAACCAACACAGACCCTG
TCCATCACATGTACTGTTTCTGGGTTCTCATTAACCAGCTATTATATACAGTGGGTTC
GCCAGACTCCAGGAAAGGGACTAGAATGGATGGGATTTATACGGAGTGGTGGAAG
CACAGAGTATAATTCAGAGTTCAAATCCCGACTTAGCATCAACAGGGACACCTCC
AAGAACCAAGTTTTCTTAAAAATGAACAGTCTGAAAACAGAGGACACAGGCGTG
TACTACTGTGCCAGAACCTCTTCGGGGTACGAAGGGGGTTTTGATTACTGGGGCC
AAGGAGTCATGGTCACAGTCTCCTCA
The nucleotide sequence of SEQ ID NO: 6 after codon optimization is shown in <SEQ ID
NO: 12>.
ATGGCAATCCTCGTGTTGCTTCTGTGCTTGGTGACCATTCCACACTCTGTGCTTTCC
CAGGTGCAGCTCAAGGAAACAGGGCCAGGACTCGTCCAACCTACACAAACCCTG
TCAATCACCTGTACCGTATCCGGTTTTAGCCTCACCAGCTATTATATACAATGGGTG
AGGCAGACCCCCGGGAAAGGACTGGAATGGATGGGCTTCATTCGCAGCGGTGGG
AGTACCGAGTACAATAGCGAGTTTAAAAGTCGCTTGAGTATCAATAGAGATACTTC
CAAGAATCAGGTGTTCTTGAAGATGAACTCCCTCAAGACCGAAGATACAGGGGTC
TATTACTGCGCCAGGACCTCCAGTGGATATGAAGGAGGCTTTGATTATTGGGGGCA
GGGCGTCATGGTAACTGTGAGCTCA
<SEQ ID NO: 7>
SEQ ID NO: 7 shows the nucleotide sequence of the CL of a bovine antibody (bovine Ig
lambda, GenBank: X62917).
CAGCCCAAGTCCCCACCCTCGGTCACCCTGTTCCCGCCCTCCACGGAGGAGCTCA
ACGGCAACAAGGCCACCCTGGTGTGTCTCATCAGCGACTTCTACCCGGGTAGCGT
GACCGTGGTCTGGAAGGCAGACGGCAGCACCATCACCCGCAACGTGGAGACCAC
CCGGGCCTCCAAACAGAGCAACAGCAAGTACGCGGCCAGCAGCTACCTGAGCCT
GACGAGCAGCGACTGGAAATCGAAAGGCAGTTACAGCTGCGAGGTCACGCACGA
GGGGAGCACCGTGACGAAGACAGTGAAGCCCTCAGAGTGTTCTTAG
The nucleotide sequence of SEQ ID NO: 7 after codon optimization is shown in <SEQ ID
NO: 13>.
CAACCCAAGTCACCACCATCCGTGACTCTGTTTCCTCCATCTACAGAGGAGCTGA
ACGGAAACAAAGCTACCTTGGTGTGTCTCATCTCTGACTTTTACCCCGGATCTGTC
ACTGTGGTATGGAAAGCAGATGGCAGCACAATAACCAGGAATGTTGAAACCACAC
GAGCCTCCAAGCAGTCCAATAGTAAGTATGCCGCATCTTCATATCTGTCCCTTACAA
GCTCAGACTGGAAATCCAAAGGCAGCTACAGTTGCGAGGTCACACATGAAGGCA
GCACCGTGACAAAGACCGTAAAGCCATCTGAGTGTAGCTAG
<SEQ ID NO: 8>
SEQ ID NO: 8 shows the nucleotide sequence (after codon optimization) of the CH of a
bovine antibody (bovine IgG1, modified from GenBank: X62916).
GCTAGCACCACAGCACCTAAAGTTTACCCTCTGTCTTCCTGCTGCGGCGACAAGT
CTTCATCAACTGTTACTCTTGGATGCCTGGTCTCAAGTTACATGCCCGAGCCCGTG
ACAGTGACCTGGAACTCAGGCGCTCTGAAGTCTGGAGTGCACACATTTCCAGCTG
TGCTTCAGTCTAGCGGCCTGTATTCCCTCAGCTCTATGGTTACTGTACCTGGTAGCA
CCAGCGGACAGACTTTCACCTGTAATGTTGCCCATCCCGCATCTTCTACCAAGGTC
GATAAAGCCGTTGACCCCACTTGCAAACCATCCCCTTGTGATTGTTGTCCACCCCC
TCCAGTGGCTGGCCCTTCCGTCTTCATTTTCCCTCCTAAACCTAAGGATACTCTGAC
CATCTCAGGGACACCCGAGGTCACCTGTGTCGTCGTGGACGTGGGACATGACGAC
CCAGAAGTCAAGTTCTCATGGTTCGTGGACGATGTGGAGGTGAACACAGCAACA
ACAAAGCCCAGAGAAGAACAGTTTAACAGCACATATCGGGTGGTCAGCGCCTTGC
GTATTCAGCACCAGGACTGGACTGGTGGCAAGGAGTTTAAGTGCAAGGTGCATAA
CGAAGGTCTGCCCTCTTCTATAGTGAGAACTATCTCCCGAACTAAGGGCCCCGCTC
GGGAGCCCCAGGTTTACGTCCTTGCTCCCCCTCAGGAGGAACTGAGTAAATCAAC
CGTGAGTCTCACCTGTATGGTTACCTCATTTTACCCAGACTACATCGCCGTAGAGT
GGCAGAGGAATGGACAGCCAGAGTCTGAGGACAAATACGGCACTACTCCTCCCC
AACTGGATGCCGACTCTTCCTACTTCCTCTACTCCAAATTGCGAGTTGACCGGAAC
TCATGGCAGGAGGGGGACACATACACATGCGTCGTTATGCACGAGGCCCTGCACA
ACCATTACACCCAGAAGTCCACATCTAAAAGTGCAGGTAAGTAA
<SEQ ID NO: 9>
SEQ ID NO: 9 shows the amino acid sequence of a chimeric light chain consisting of
the VL of a rat anti-bovine PD-1 antibody and the CL of a bovine antibody.
MKVPGRLLVLLFWIPASRSDVVLTQTPVSLSVFLGDQASISCRSSQSLEYSDGYTYLE
WYLQKPGQSPQLLIYGVSNRFSGVPDRFIGSGSGTDFTLKISRVEPEDLGVYYCFQAT
HDPDTFGAGTKLELKQPKSPPSVTLFPPSTEELNGNKATLVCLISDFYPGSVTVVWKA
DGSTITRNVETTRASKQSNSKYAASSYLSLTSSDWKSKGSYSCEVTHEGSTVFKFVKP
SECS
<SEQ ID NO: 10>
SEQ ID NO: 10 shows the amino acid sequence of a chimeric heavy chain consisting of the
VH of a rat anti-bovine PD-1 antibody and the CH of a bovine antibody (bovine IgG1,
modified from GenBank: X62916),
MAILVLLLCLVTIPHSVLSQVQLKETGPGLVQPTQTLSITCTVSGFSLTSYYIQWVRQT
PGKGLEWMGFIRSGGSTEYNSEFKSRLSINRDTSKNQVFLKMNSLKTEDTGVYYCAR
TSSGYEGGFDYWGQGVMVTVSSASTTAPKVYPLSSCCGDKSSSTVTLGCLVSSYMPE
PVTVTWNSGALKSGVHTFPAVLQSSGLYSLSSMVTVPGSTSGQTFTCNVAHPASSTKV
DKAVDPTCKPSPCDCCPPPPVAGPSVFIFPPKPKDTLTISGTPEVTCVVVDVGHDDPEV
KFSWFVDDVEVNTATTKPREEQFNSTYRVVSALRIQHQDWTGGKEFKCKVHNEGLP
SSIVRTISRTKGPAREPQVYVLAPPQEELSKSTVSLTCMVTSFYPDYIAVEWQRNGQPE
SEDKYGITPPQLDADSSYFLYSKLRVDRNSWQEGDTYTCVVMHEALHNHYTQKSTS
KSAGK
<SEQ ID NO: 14>
SEQ ID NO: 14 shows the nucleotide sequence (after codon optimization) of a chimeric light
chain consisting of the VL of a rat anti-bovine PD-1 antibody and the CL of a bovine
antibody.
ATGAAGGTCCCTGGTAGGCTGCTGGTTCTCTTGTTCTGGATCCCTGCTTCCAGAAG
TGACGTGGTGCTGACTCAAACACCAGTGAGTCTCAGTGTGACCCTCGGCGACCA
GGCCTCCATTTCTTGCCGTAGCAGCCAGTCCTTGGAATACTCTGATGGTTATACTTA
TCTGGAGTGGTACCTCCAGAAGCCCGGGCAGTCACCCCAGCTTCTTATCTACGGT
GTGAGCAACAGATTTTCTGGGGTTCCTGATCGGTTTATTGGATCTGGATCCGGTAC
CGACTTCACATTGAAAATTTCACGCGTCGAACCCGAGGATCTGGGGGTCTACTATT
GCTTCCAAGCCACCCACGATCCCGACACCTTCGGCGCTGGCACTAAGCTGGAGCT
GAAACAACCCAAGTCACCACCATCCGTGACTCTGTTTCCTCCATCTACAGAGGAG
CTGAACGGAAACAAAGCTACCTTGGTGTGTCTCATCTCTGACTTTTACCCCGGATC
TGTCACTGTGGTATGGAAAGCAGATGGCAGCACAATAACCAGGAATGTTGAAACC
ACACGAGCCTCCAAGCAGTCCAATAGTAAGTATGCCGCATCTTCATATCTGTCCCTT
ACAAGCTCAGACTGGAAATCCAAAGGCAGCTACAGTTGCGAGGTCACACATGAA
GGCAGCACCGTGACAAAGACCGTAAAGCCATCTGAGTGTAGCTAG
<SEQ ID NO: 15>
SEQ ID NO: 15 shows the nucleotide sequence (after codon optimization) of a chimeric
heavy chain consisting of the VH of a rat anti-bovine PD-1 antibody and the CH of a bovine
antibody (bovine IgG1, modified from GenBank: X62916).
ATGGCAATCCTCGTGTTGCTTCTGTGCTTGGTGACCATTCCACACTCTGTGCTTTCC
CAGGTGCAGCTCAAGGAAACAGGGCCAGGACTCGTCCAACCTACACAAACCCTG
TCAATCACCTGTACCGTATCCGGTTTTAGCCTCACCAGCTATTATATACAATGGGTG
AGGCAGACCCCCGGGAAAGGACTGGAATGGATGGGCTTCATTCGCAGCGGTGGG
AGTACCGAGTACAATAGCGAGTTTAAAAGTCGCTTGAGTATCAATAGAGATACTTC
CAAGAATCAGGTGTTCTTGAAGATGAACTCCCTCAAGACCGAAGATACAGGGGTC
TATTACTGCGCCAGGACCTCCAGTGGATATGAAGGAGGCTTTGATTATTGGGGGCA
GGGCGTCATGGTAACTGTGAGCTCAGCTAGCACCACAGCACCTAAAGTTTACCCT
CTGTCTTCCTGCTGCGGCGACAAGTCTTCATCAACTGTTACTCTTGGATGCCTGGT
CTCAAGTTACATGCCCGAGCCCGTGACAGTGACCTGGAACTCAGGCGCTCTGAAG
TCTGGAGTGCACACATTTCCAGCTGTGCTTCAGTCTAGCGGCCTGTATTCCCTCAG
CTCTATGGTTACTGTACCTGGTAGCACCAGCGGACAGACTTTCACCTGTAATGTrG
CCCATCCCGCATCTTCTACCAAGGTCGATAAAGCCGTTGACCCCACTTGCAAACCA
TCCCCTTGTGATTGTTGTCCACCCCCTCCAGTGGCTGGCCCTTCCGTCTTCATTTTC
CCTCCTAAACCTAAGGATACTCTGACCATCTCAGGGACACCCGAGGTCACCTGTGT
CGTCGTGGACGTGGGACATGACGACCCAGAAGTCAAGTTCTCATGGTTCGTGGAC
GATGTGGAGGTGAACACAGCAACAACAAAGCCCAGAGAAGAACAGTTTAACAGC
ACATATCGGGTGGTCAGCGCCTTGCGTATTCAGCACCAGGACTGGACTGGTGGCA
AGGAGTTTAAGTGCAAGGTGCATAACGAAGGTCTGCCCTCTTCTATAGTGAGAACT
ATCTCCCGAACTAAGGGCCCCGCTCGGGAGCCCCAGGTTTACGTCCTTGCTCCCC
CTCAGGAGGAACTGAGTAAATCAACCGTGAGTCTCACCTGTATGGTTACCTCATTT
TACCCAGACTACATCGCCGTAGAGTGGCAGAGGAATGGACAGCCAGAGTCTGAG
GACAAATACGGCACTACTCCTCCCCAACTGGATGCCGACTCTTCCTACTTCCTCTA
CTCCAAATTGCGAGTTGACCGGAACTCATGGCAGGAGGGGGACACATACACATGC
GTCGTTATGCACGAGGCCCTGCACAACCATTACACCCAGAAGTCCACATCTAAAA
GTGCAGGTAAGTAA
<SEQ ID NO: 16>
SEQ ID NO: 16 shows the amino acid sequence (QSLEYSDGYTY) of CDR1 of the VL of
rat anti-bovine PD-1 antibody 5D2.
<SEQ ID NO: 17>
SEQ ID NO: 17 shows the amino acid sequence (FQATHDPDT) of CDR3 of the VL of rat
anti-bovine PD-1 antibody 5D2.
<SEQ ID NO: 18>
SEQ ID NO: 18 shows the amino acid sequence (GFSLTSYY) of CDR1 of the VH of rat
anti-bovine PD-1 antibody 5D2.
<SEQ ID NO: 19>
SEQ ID NO: 19 shows the amino acid sequence (IRSGGST) of CDR2 of the VH of rat anti-
bovine PD-1 antibody 5D2.
<SEQ ID NO: 20>
SEQ ID NO: 20 shows the amino acid sequence (ARTSSGYEGGFDY) of CDR3 of the VH
of rat anti-bovine PD-1 antibody 5D2.
<SEQ ID NO: 21>
SEQ ID NO: 21 shows the amino acid sequence of the CH (CH1-CH3) of a bovine antibody
(IgG1 variant 1).
<SEQ ID NO: 22>
SEQ ID NO: 22 shows the amino acid sequence of the CH (CH1-CH3) of a bovine antibody
(IgG1 variant 2).
<SEQ ID NO: 23>
SEQ ID NO: 23 shows the amino acid sequence of the CH (CH1-CH3) of a bovine antibody
(IgG1 variant 3).
<SEQ ID NO: 24>
SEQ ID NO: 24 shows the amino acid sequence of the CH (CH1-CH3) of a bovine antibody
(IgG2 variant 1).
<SEQ ID NO: 25>
SEQ ID NO: 25 shows the amino acid sequence of the CH (CH1-CH3) of a bovine antibody
(IgG2 variant 2).
<SEQ ID NO: 26>
SEQ ID NO: 26 shows the amino acid sequence of the CH (CH1-CH3) of a bovine antibody
(IgG2 variant 3).
<SEQ ID NO: 27>
SEQ ID NO: 27 shows the amino acid sequence of the CH (CH1-CH3) of a bovine antibody
(IgG3 variant 1).
<SEQ ID NO: 28>
SEQ ID NO: 28 shows the amino acid sequence of the CH (CH1-CH3) of a bovine antibody
(IgG3 variant 2).
<SEQ ID NO: 29>
SEQ ID NO: 29 shows the nucleotide sequence of the CH (CH1-CH3) of a bovine antibody
(IgG1 variant 1).
<SEQ ID NO: 30>
SEQ ID NO: 30 shows the nucleotide sequence of the CH (CH1-CH3) of a bovine antibody
(IgG1 variant 2).
<SEQ ID NO: 31>
SEQ ID NO: 31 shows the nucleotide sequence of the CH (CH1-CH3) of a bovine antibody
(IgG1 variant 3).
<SEQ ID NO: 32>
SEQ ID NO: 32 shows the nucleotide sequence of the CH (CH1-CH3) of a bovine antibody
(IgG2 variant 1).
<SEQ ID NO: 33>
SEQ ID NO: 33 shows the nucleotide sequence of the CH (CH1-CH3) of a bovine antibody
(IgG2 variant 2).
<SEQ ID NO: 34>
SEQ ID NO: 34 shows the nucleotide sequence of the CH (CH1-CH3) of a bovine antibody
(IgG2 variant 3).
<SEQ ID NO: 35>
SEQ ID NO: 35 shows the nucleotide sequence of the CH (CH1-CH3) of a bovine antibody
(IgG3 variant 1).
<SEQ ID NO: 36>
SEQ ID NO: 36 shows the nucleotide sequence of the CH (CH1-CH3) of a bovine antibody
(IgG3 variant 2).
<SEQ ID NO: 37>
SEQ ID NO: 37 shows the amino acid sequence of the CH (CH1-CH3) of an ovine antibody
(IgG1).
<SEQ ID NO: 38>
SEQ ID NO: 38 shows the nucleotide sequence of the CH (CH1-CH3) of an ovine antibody
(IgG1).
<SEQ ID NO: 39>
SEQ ID NO: 39 shows the amino acid sequence of the CH (CH1-CH3) of an ovine antibody
(IgG2).
<SEQ ID NO: 40>
SEQ ID NO: 40 shows the nucleotide sequence of the CH (CH1-CH3) of an ovine antibody
(IgG2).
<SEQ ID NO: 41>
SEQ ID NO: 41 shows the amino acid sequence of the light chain (Ig kappa (CK)) constant
region of an ovine antibody.
<SEQ ID NO: 42>
SEQ ID NO: 42 shows the nucleotide sequence of the light chain (Ig kappa (CK)) constant
region of an ovine antibody.
<SEQ ID NO: 43>
SEQ ID NO: 43 shows the amino acid sequence of the light chain (Ig lambda (CL)) constant
region of an ovine antibody.
<SEQ ID NO: 44>
SEQ ID NO: 44 shows the nucleotide sequence of the light chain (Ig lambda (CL)) constant
region of an ovine antibody.
<SEQ ID NO: 45>
SEQ ID NO: 45 shows the amino acid sequence of the CH (CH1-CH3) of a water buffalo
antibody (presumed to be IgG1).
<SEQ ID NO: 46>
SEQ ID NO: 46 shows the nucleotide sequence of the CH (CH1-CH3) of a water buffalo
antibody (presumed to be IgG1).
<SEQ ID NO: 47>
SEQ ID NO: 47 shows the amino acid sequence of the CH (CH1-CH3) of a water buffalo
antibody (presumed to be IgG2).
<SEQ ID NO: 48>
SEQ ID NO: 48 shows the nucleotide sequence of the CH (CH1-CH3) of a water buffalo
antibody (presumed to be IgG2).
<SEQ ID NO: 49>
SEQ ID NO: 49 shows the amino acid sequence of the CH (CH1-CH3) of a water buffalo
antibody (presumed to be IgG3).
<SEQ ID NO: 50>
SEQ ID NO: 50 shows the nucleotide sequence of the CH (CH1-CH3) of a water buffalo
antibody (presumed to be IgG3).
<SEQ ID NO: 51>
SEQ ID NO: 51 shows the amino acid sequence of the constant region of the light chain
(presumed to be Ig lambda) of a water buffalo antibody.
<SEQ ID NO: 52>
SEQ ID NO: 52 shows the nucleotide sequence of the constant region of the light chain
(presumed to be Ig lambda) of a water buffalo antibody,
<SEQ ID NO: 53>
SEQ ID NO: 53 shows the amino acid sequence of the CH (CH1-CH3) of a human antibody
(IgG4 variant 1).
<SEQ ID NO: 54>
SEQ ID NO: 54 shows the nucleotide sequence of the CH (CH1-CH3) of a human antibody
(IgG4 variant 1).
<SEQ ID NO: 55>
SEQ ID NO: 55 shows the amino acid sequence of the CH (CH1-CH3) of a human antibody
(IgG4 variant 2).
<SEQ ID NO: 56>
SEQ ID NO: 56 shows the nucleotide sequence of the CH (CH1-CH3) of a human antibody
(IgG4 variant 2).
<SEQ ID NO: 57>
SEQ ID NO: 57 shows the amino acid sequence of the CH (CH1-CH3) of a human antibody
(IgG4 variant 3).
<SEQ ID NO: 58>
SEQ ID NO: 58 shows the nucleotide sequence of the CH (CH1-CH3) of a human antibody
(IgG4 variant 31).
<SEQ ID NO: 59>
SEQ ID NO: 59 shows the amino acid sequence of the CL of a human antibody.
<SEQ ID NO: 60>
SEQ ID NO: 60 shows the nucleotide sequence of the CL of a human antibody.
<SEQ ID NOS: 61-74>
SEQ ID NOS: 61-74 show the nucleotide sequences of primers boPD-1-myc F, boPD-1-myc
R, boPD-L1-EGFP F, boPD-L1-EGFP R, boPD-1-His F, boPD-1-His R, ovPD-1 CDS F,
ovPD-1 CDS R, buPD-1 CDS F1, buPD-1 CDS R1, buPD-1 CDS F2, buPD-1 CDS R2,
ovPD-1-EGFP F and ovPD-1-EGFP R in this order.
<SEQ ID NO: 75>
SEQ ID NO: 75 shows the nucleotide sequence of a chimeric heavy chain consisting of the
VH of a rat anti-bovine PD-1 antibody and the CH of a bovine antibody (bovine IgG1,
GenBank: X62916).
ATGGCAATCCTCGTGTTGCTTCTGTGCTTGGTGACCATTCCACACTCTGTGCTTTCC
CAGGTGCAGCTCAAGGAAACAGGGCCAGGACTCGTCCAACCTACACAAACCCTG
TCAATCACCTGTACCGTATCCGGTTTTAGCCTCACCAGCTATTATATACAATGGGTG
AGGCAGACCCCCGGGAAAGGACTGGAATGGATGGGCTTCATTCGCAGCGGTGGG
AGTACCGAGTAGAATAGCGAGTTTAAAAGTCGCTTGAGTATCAATAGAGATACTTC
CAAGAATCAGGTGTTCTTGAAGATGAACTCCCTCAAGACCGAAGATACAGGGGTC
TATTACTGCGCCAGGACCTCCAGTGGATATGAAGGAGGCTTTGATTATTGGGGGCA
GGGCGTCATGGTAACTGTGAGCTCAGCCTCCACCACAGCCCCGAAAGTCTACCCT
CTGAGTTCTTGCTGCGGGGACAAGTCCAGCTCCACCGTGACCCTGGGCTGCCTGG
TCTCCAGCTACATGCCCGAGCCGGTGACCGTGACCTGGAACTCGGGTGCCCTGAA
GAGCGGCGTGCACACCTTCCCGGCTGTCCTTCAGTCCTCCGGGCTGTACTCTCTCA
GCAGCATGGTGACCGTGCCCGGCAGCACCTCAGGACAGACCTTCACCTGCAACGT
AGCCCACCCGGCCAGCAGCACCAAGGTGGACAAGGCTGTTGATCCCACATGCAA
ACCATCACCCTGTGACTGTTGCCCACCCCCTGAGCTCCCCGGAGGACCCTCTGTC
TTCATCTTCCCACCGAAACCCAAGGACACCCTCACAATCTCGGGAACGCCCGAGG
TCACGTGTGTGGTGGTGGACGTGGGCCACGATGACCCCGAGGTGAAGTTCTCCTG
GTTCGTGGACGACGTGGAGGTAAACACAGCCACGACGAAGCCGAGAGAGGAGC
AGTTCAACAGCACCTACCGCGTGGTCAGCGCCCTGCGCATCCAGCACCAGGACTG
GACTGGAGGAAAGGAGTTCAAGTGCAAGGTCCACAACGAAGGCCTCCCGGCCCC
CATCGTGAGGACCATCTCCAGGACCAAAGGGCCGGCCCGGGAGCCGCAGGTGTAT
GTCCTGGCCCCACCCCAGGAAGAGCTGAGCAAAAGCACGGTCAGCCTCACCTGC
ATGGTCACCAGCTTCTACCCAGACTACATCGCCGTGGAGTGGCAGAGAAACGGGC
AGCCTGAGTCGGAGGACAAGTACGGCACGACCCCGCCCCAGCTGGACGCCGACA
GCTCCTACTTCCTGTACAGCAAGCTCAGGGTGGACAGGAACAGCTGGCAGGAAG
GAGACACCTACACGTGTGTGGTGATGCACGAGGCCCTGCACAATCACTACACGCA
GAAGTCCACCTCTAAGTCTGCGGGTAAATAA
<SEQ ID NO: 76>
SEQ ID NO: 76 shows the amino acid sequence of a chimeric heavy chain consisting of the
VH of a rat anti-bovine PD-1 antibody and the CH of a bovine antibody (bovine IgG1,
GenBank: X62916).
MAILVLLLCLVTIPHSVLSQVQLKETGPGLVQPTQTLSITCTVSGFSLTSYYIQWVRQT
PGKGLEWMGFIRSGGSTEYNSEFKSRLSINRDTSKNQVFLKMNSLKTEDTGVYYCAR
TSSGYEGGFDYWGQGVMVTVSSASTTAPKVYPLSSCCGDKSSSTVTLGCLVSSYMPE
PVTVTWNSGALKSGYHTFPAVLQSSGLYSLSSMVTVPGSTSGQTFTCNVAHPASSTKV
DKAVDPTCKPSPCDCCPPPELPGGPSVFIFPPKPKDTLTISGTPEVTCVVVDVGHDDPE
VKFSWFVDDVEVNTATTKPREEQFNSTYRVVSALRIQHQDWTGGKEFKCKVHNEGL
PAPIVRTISRTKGPAREPQVYVLAPPQEELSKSTVSLTCMVTSFYPDYIAVEWQRNGQP
ESEDKYGTTPPQLDADSSYFLYSKLRVDRNSWQEGDTYTCVVMHEALHNHYTQKST
SKSAGK
<SEQ ID NOS: 77-86>
SEQ ID NOS: 77-86 show the nucleotide sequences of primers boIgG1 CH1 F, boIgG1 CH3
R, boFcγRI-His F, boFcγRI-His R, boFcγRII-His F, boFcγRII-His R, boFcγRIII-His F,
boFcγRIII-His R, boFcγ2R-His F and boFcγ2R-His R in this order.

1. An anti-PD-1 antibody comprising (a) a light chain comprising a light chain variable region containing CDR1 having the amino acid sequence of QSLEYSDGYTY (SEQ ID NO: 16), CDR2 having the amino acid sequence of GVS and CDR3 having the amino acid sequence of FQATHDPDT (SEQ ID NO: 17) and the light chain constant region of an antibody of an animal other than rat; and (b) a heavy chain comprising a heavy chain variable region containing CDR1 having the amino acid sequence of GFSLTSYY (SEQ ID NO: 18), CDR2 having the amino acid sequence of IRSGGST (SEQ ID NO: 19) and CDR3 having the amino acid sequence of ARTSSGYEGGFDY (SEQ II) NO: 20) and the heavy chain constant region of an antibody of an animal other than rat.

2. The antibody of claim 1, wherein the light chain variable region and the heavy chain variable region are derived from rat.

3. The antibody of claim 2, wherein the light chain variable region is the light chain variable region of a rat anti-bovine PD-1 antibody and the heavy chain variable region is the heavy chain variable region of a rat anti-bovine PD-1 antibody.

4. The antibody of claim 3, wherein the light chain variable region has the amino acid sequence as shown in SEQ ID NO. 1 and the heavy chain variable region has the amino acid sequence as shown in SEQ ID NO: 2.

5. The antibody of claim 1, wherein the light chain constant region of an antibody of an animal other than rat has the amino acid sequence of the constant region of lambda chain or kappa chain.

6. The antibody of claim 1, wherein the heavy chain constant region of an antibody of an animal other than rat has the amino acid sequence of the constant region of an immunoglobulin equivalent to human IgG4, or has mutations introduced thereinto that reduce ADCC activity and/or CDC activity.

7. The antibody of claim 6, wherein the animal other than rat is bovine; the light chain constant region of the bovine antibody has the amino acid sequence of the constant region of lambda chain; and the heavy chain constant region of the bovine antibody has mutations introduced thereinto that reduce ADCC activity and/or CDC activity.

8. The antibody of claim 7, wherein the light chain constant region of the bovine antibody has the amino acid sequence as shown in SEQ ID NO: 3 and the heavy chain constant region of the bovine antibody has the amino acid sequence as shown in SEQ ID NO: 4

9. The antibody of claim 1 which has a four-chain structure comprising two light chains and two heavy chains.

10. A pharmaceutical composition comprising the antibody of claim 1 as an active ingredient.

11. The pharmaceutical composition of claim 10 for prevention and/or treatment of cancers and/or inflammations.

12. The pharmaceutical composition of claim 11, wherein the cancers and/or inflammations are selected from the group consisting of neoplastic diseases, leukemia, Johne's disease, anaplasmosis, bacterial mastitis, mycotic mastitis, mycoplasma infections (such as mycoplasma mastitis, mycoplasma pneumonia or the like), tuberculosis, Theileria orientalis infection, cryptosporidiosis, coccidiosis, trypanosomiasis and leishmaniasis.

13. An artificial genetic DNA comprising (a′) a DNA encoding a light chain comprising a light chain variable region containing CDR1 having the amino acid sequence of QSLEYSDGYTY (SEQ ID NO: 16), CDR2 having the amino acid sequence of GVS and CDR3 having the amino acid sequence of FQATHDPDT (SEQ ID NO: 17) and the light chain constant region of an antibody of an animal other than rat and (b′) a DNA encoding a heavy chain comprising a heavy chain variable region containing CDR1 having the amino acid sequence of GFSLTSYY (SEQ ID NO: 18), CDR2 having the amino acid sequence of IRSGGST (SEQ ID NO: 19) and CDR3 having the amino acid sequence of ARTSSGYEGGFDY (SEQ ID NO: 20) and the heavy chain constant region of an antibody of an animal other than rat.

14. A vector comprising the artificial genetic DNA of claim 13.

15. A host cell transformed with the vector of claim 14.

16. A method of preparing an antibody, comprising culturing the host cell of claim 15 and collecting an anti-PD-1 antibody from the resultant culture.

17. A DNA encoding a light chain comprising a light chain variable region containing CDR1 having the amino acid sequence of QSLEYSDGYTY (SEQ ID NO: 16), CDR2 having the amino acid sequence of GVS and CDR3 having the amino acid sequence of FQATHDPDT (SEQ ID NO: 17) and the light chain constant region of an antibody of an animal other than rat.

18. A DNA encoding a heavy chain comprising a heavy chain variable region containing CDR1 having the amino acid sequence of GFSLTSYY (SEQ ID NO: 18), CDR2 having the amino acid sequence of IRSGGST (SEQ ID NO: 19) and CDR3 having the amino acid sequence of ARTSSGYEGGFDY (SEQ ID NO: 20) and the heavy chain constant region of an antibody of an animal other than rat.


SEQUENCE LISTING (86 sequences)

SEQ ID NO: 1 - protein, 131 aa, Rattus norvegicus
MKVPGRLLVLLFWIPASRSDVVLTQTPVSLSVTLGDQASISCRSSQSLEYSDGYTYLEWYLQKPGQSPQLLIYGVSNRFSGVPDRFIGSGSGTDFTLKISRVEPEDLGVYYCFQATHDPDTFGAGTKLELK

SEQ ID NO: 2 - protein, 138 aa, Rattus norvegicus
MAILVLLLCLVTIPHSVLSQVQLKETGPGLVQPTQTLSITCTVSGFSLTSYYIQWVRQTPGKGLEWMGFIRSGGSTEYNSEFKSRLSINRDTSKNQVFLKMNSLKTEDTGVYYCARTSSGYEGGFDYWGQGVMVTVSS

SEQ ID NO: 3 - protein, 105 aa, Bos taurus
QPKSPPSVTLFPPSTEELNGNKATLVCLISDFYPGSVTVVWKADGSTITRNVETTRASKQSNSKYAASSYLSLTSSDWKSKGSYSCEVTHEGSTVTKTVKPSECS

SEQ ID NO: 4 - protein, 328 aa, Bos taurus
ASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVDPTCKPSPCDCCPPPPVAGPSVFIFPPKPKDTLTISGTPEVTCVVVDVGHDDPEVKFSWFVDDVEVNTATTKPREEQFNSTYRVVSALRIQHQDWTGGKEFKCKVHNEGLPSSIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 5 - DNA, 393 bp, Rattus norvegicus
atgaaagtgcctggtaggctgctggtgctgttgttttggattccagcttccaggagtgatgttgtgttgacacaaactccagtttccctgtctgtcacacttggagatcaagcttctatatcttgcaggtctagtcagagcctggaatatagtgatggatacacttatttggaatggtacctacagaagccaggccagtctccacagctcctcatctatggagtttccaaccgattttctggggtcccagacaggttcattggcagtgggtcagggacagatttcaccctcaagatcagcagagtagagcctgaggacttgggagtttattactgcttccaagctacacatgatccggacacgtttggagctgggaccaagctggaactgaaa

SEQ ID NO: 6 - DNA, 414 bp, Rattus norvegicus
atggctatcctggtgctgcttctctgcctggtgaccattccacactctgtcttgtcccaggtgcagctgaaggagacaggacctggcctggtgcaaccaacacagaccctgtccatcacatgtactgtttctgggttctcattaaccagctattatatacagtgggttcgccagactccaggaaagggactagaatggatgggatttatacggagtggtggaagcacagagtataattcagagttcaaatcccgacttagcatcaacagggacacctccaagaaccaagttttcttaaaaatgaacagtctgaaaacagaggacacaggcgtgtactactgtgccagaacctcttcggggtacgaagggggttttgattactggggccaaggagtcatggtcacagtctcctca

SEQ ID NO: 7 - DNA, 318 bp, Bos taurus
cagcccaagtccccaccctcggtcaccctgttcccgccctccacggaggagctcaacggcaacaaggccaccctggtgtgtctcatcagcgacttctacccgggtagcgtgaccgtggtctggaaggcagacggcagcaccatcacccgcaacgtggagaccacccgggcctccaaacagagcaacagcaagtacgcggccagcagctacctgagcctgacgagcagcgactggaaatcgaaaggcagttacagctgcgaggtcacgcacgaggggagcaccgtgacgaagacagtgaagccctcagagtgttcttag

SEQ ID NO: 8 - DNA, 987 bp, Artificial Sequence
  codon-optimized sequence
gctagcaccacagcacctaaagtttaccctctgtcttcctgctgcggcgacaagtcttcatcaactgttactcttggatgcctggtctcaagttacatgcccgagcccgtgacagtgacctggaactcaggcgctctgaagtctggagtgcacacatttccagctgtgcttcagtctagcggcctgtattccctcagctctatggttactgtacctggtagcaccagcggacagactttcacctgtaatgttgcccatcccgcatcttctaccaaggtcgataaagccgttgaccccacttgcaaaccatccccttgtgattgttgtccaccccctccagtggctggcccttccgtcttcattttccctcctaaacctaaggatactctgaccatctcagggacacccgaggtcacctgtgtcgtcgtggacgtgggacatgacgacccagaagtcaagttctcatggttcgtggacgatgtggaggtgaacacagcaacaacaaagcccagagaagaacagtttaacagcacatatcgggtggtcagcgccttgcgtattcagcaccaggactggactggtggcaaggagtttaagtgcaaggtgcataacgaaggtctgccctcttctatagtgagaactatctcccgaactaagggccccgctcgggagccccaggtttacgtccttgctccccctcaggaggaactgagtaaatcaaccgtgagtctcacctgtatggttacctcattttacccagactacatcgccgtagagtggcagaggaatggacagccagagtctgaggacaaatacggcactactcctccccaactggatgccgactcttcctacttcctctactccaaattgcgagttgaccggaactcatggcaggagggggacacatacacatgcgtcgttatgcacgaggccctgcacaaccattacacccagaagtccacatctaaaagtgcaggtaagtaa

SEQ ID NO: 9 - protein, 236 aa, Artificial Sequence
  chimeric L chain
MKVPGRLLVLLFWIPASRSDVVLTQTPVSLSVTLGDQASISCRSSQSLEYSDGYTYLEWYLQKPGQSPQLLIYGVSNRFSGVPDRFIGSGSGTDFTLKISRVEPEDLGVYYCFQATHDPDTFGAGTKLELKQPKSPPSVTLFPPSTEELNGNKATLVCLISDFYPGSVTVVWKADGSTITRNVETTRASKQSNSKYAASSYLSLTSSDWKSKGSYSCEVTHEGSTVTKTVKPSECS

SEQ ID NO: 10 - protein, 466 aa, Artificial Sequence
  chimeric H chain
MAILVLLLCLVTIPHSVLSQVQLKETGPGLVQPTQTLSITCTVSGFSLTSYYIQWVRQTPGKGLEWMGFIRSGGSTEYNSEFKSRLSINRDTSKNQVFLKMNSLKTEDTGVYYCARTSSGYEGGFDYWGQGVMVTVSSASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVDPTCKPSPCDCCPPPPVAGPSVFIFPPKPKDTLTISGTPEVTCVVVDVGHDDPEVKFSWFVDDVEVNTATTKPREEQFNSTYRVVSALRIQHQDWTGGKEFKCKVHNEGLPSSIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 11 - DNA, 393 bp, Artificial Sequence
  codon-optimzed sequence
atgaaggtccctggtaggctgctggttctcttgttctggatccctgcttccagaagtgacgtggtgctgactcaaacaccagtgagtctcagtgtgaccctcggcgaccaggcctccatttcttgccgtagcagccagtccttggaatactctgatggttatacttatctggagtggtacctccagaagcccgggcagtcaccccagcttcttatctacggtgtgagcaacagattttctggggttcctgatcggtttattggatctggatccggtaccgacttcacattgaaaatttcacgcgtcgaacccgaggatctgggggtctactattgcttccaagccacccacgatcccgacaccttcggcgctggcactaagctggagctgaaa

SEQ ID NO: 12 - DNA, 414 bp, Artificial Sequence
  codon-optimized sequence
atggcaatcctcgtgttgcttctgtgcttggtgaccattccacactctgtgctttcccaggtgcagctcaaggaaacagggccaggactcgtccaacctacacaaaccctgtcaatcacctgtaccgtatccggttttagcctcaccagctattatatacaatgggtgaggcagacccccgggaaaggactggaatggatgggcttcattcgcagcggtgggagtaccgagtacaatagcgagtttaaaagtcgcttgagtatcaatagagatacttccaagaatcaggtgttcttgaagatgaactccctcaagaccgaagatacaggggtctattactgcgccaggacctccagtggatatgaaggaggctttgattattgggggcagggcgtcatggtaactgtgagctca

SEQ ID NO: 13 - DNA, 318 bp, Artificial Sequence
  codon-optimized sequence
caacccaagtcaccaccatccgtgactctgtttcctccatctacagaggagctgaacggaaacaaagctaccttggtgtgtctcatctctgacttttaccccggatctgtcactgtggtatggaaagcagatggcagcacaataaccaggaatgttgaaaccacacgagcctccaagcagtccaatagtaagtatgccgcatcttcatatctgtcccttacaagctcagactggaaatccaaaggcagctacagttgcgaggtcacacatgaaggcagcaccgtgacaaagaccgtaaagccatctgagtgtagctag

SEQ ID NO: 14 - DNA, 711 bp, Artificial Sequence
  codon-optimized sequence
atgaaggtccctggtaggctgctggttctcttgttctggatccctgcttccagaagtgacgtggtgctgactcaaacaccagtgagtctcagtgtgaccctcggcgaccaggcctccatttcttgccgtagcagccagtccttggaatactctgatggttatacttatctggagtggtacctccagaagcccgggcagtcaccccagcttcttatctacggtgtgagcaacagattttctggggttcctgatcggtttattggatctggatccggtaccgacttcacattgaaaatttcacgcgtcgaacccgaggatctgggggtctactattgcttccaagccacccacgatcccgacaccttcggcgctggcactaagctggagctgaaacaacccaagtcaccaccatccgtgactctgtttcctccatctacagaggagctgaacggaaacaaagctaccttggtgtgtctcatctctgacttttaccccggatctgtcactgtggtatggaaagcagatggcagcacaataaccaggaatgttgaaaccacacgagcctccaagcagtccaatagtaagtatgccgcatcttcatatctgtcccttacaagctcagactggaaatccaaaggcagctacagttgcgaggtcacacatgaaggcagcaccgtgacaaagaccgtaaagccatctgagtgtagctag

SEQ ID NO: 15 - DNA, 1401 bp, Artificial Sequence
  codon-optimized sequence
atggcaatcctcgtgttgcttctgtgcttggtgaccattccacactctgtgctttcccaggtgcagctcaaggaaacagggccaggactcgtccaacctacacaaaccctgtcaatcacctgtaccgtatccggttttagcctcaccagctattatatacaatgggtgaggcagacccccgggaaaggactggaatggatgggcttcattcgcagcggtgggagtaccgagtacaatagcgagtttaaaagtcgcttgagtatcaatagagatacttccaagaatcaggtgttcttgaagatgaactccctcaagaccgaagatacaggggtctattactgcgccaggacctccagtggatatgaaggaggctttgattattgggggcagggcgtcatggtaactgtgagctcagctagcaccacagcacctaaagtttaccctctgtcttcctgctgcggcgacaagtcttcatcaactgttactcttggatgcctggtctcaagttacatgcccgagcccgtgacagtgacctggaactcaggcgctctgaagtctggagtgcacacatttccagctgtgcttcagtctagcggcctgtattccctcagctctatggttactgtacctggtagcaccagcggacagactttcacctgtaatgttgcccatcccgcatcttctaccaaggtcgataaagccgttgaccccacttgcaaaccatccccttgtgattgttgtccaccccctccagtggctggcccttccgtcttcattttccctcctaaacctaaggatactctgaccatctcagggacacccgaggtcacctgtgtcgtcgtggacgtgggacatgacgacccagaagtcaagttctcatggttcgtggacgatgtggaggtgaacacagcaacaacaaagcccagagaagaacagtttaacagcacatatcgggtggtcagcgccttgcgtattcagcaccaggactggactggtggcaaggagtttaagtgcaaggtgcataacgaaggtctgccctcttctatagtgagaactatctcccgaactaagggccccgctcgggagccccaggtttacgtccttgctccccctcaggaggaactgagtaaatcaaccgtgagtctcacctgtatggttacctcattttacccagactacatcgccgtagagtggcagaggaatggacagccagagtctgaggacaaatacggcactactcctccccaactggatgccgactcttcctacttcctctactccaaattgcgagttgaccggaactcatggcaggagggggacacatacacatgcgtcgttatgcacgaggccctgcacaaccattacacccagaagtccacatctaaaagtgcaggtaagtaa

SEQ ID NO: 16 - protein, 11 aa, Rattus norvegicus
QSLEYSDGYTY

SEQ ID NO: 17 - protein, 9 aa, Rattus norvegicus
FQATHDPDT

SEQ ID NO: 18 - protein, 8 aa, Rattus norvegicus
GFSLTSYY

SEQ ID NO: 19 - protein, 7 aa, Rattus norvegicus
IRSGGST

SEQ ID NO: 20 - protein, 13 aa, Rattus norvegicus
ARTSSGYEGGFDY

SEQ ID NO: 21 - protein, 329 aa, Bos taurus
ASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVDPTCKPSPCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 22 - protein, 329 aa, Bos taurus; identical to SEQ ID NO: 21

SEQ ID NO: 23 - protein, 329 aa, Bos taurus
ASTTAPKVYPLSSCCGDKSSSTVTLGCLVSSYMPEPVTVTWNSGALKSGVHTFPAVLQSSGLYSLSSMVTVPGSTSGTQTFTCNVAHPASSTKVDKAVDPRCKTTCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADGSYFLYSRLRVDRNSWQEGDTYTCVVMHEALHNHYTQKSTSKSAGK

SEQ ID NO: 24 - protein, 326 aa, Bos taurus
ASTTAPKVYPLASSCGDTSSSTVTLGCLVSSYMPEPVTVTWNSGALKSGVHTFPAVLQSSGLYSLSSMVTVPASSSGQTFTCNVAHPASSTKVDKAVGVSIDCSKCHNQPCVREPSVFIFPPKPKDTLMITGTPEVTCVVVNVGHDNPEVQFSWFVDDVEVHTARSKPREEQFNSTYRVVSALPIQHQDWTGGKEFKCKVNNKGLSAPIVRIISRSKGPAREPQVYVLDPPKEELSKSTLSVTCMVTGFYPEDVAVEWQRNRQTESEDKYRTTPPQLDTDRSYFLYSKLRVDRNSWQEGDAYTCVVMHEALHNHYMQKSTSKSAGK

SEQ ID NO: 25 - protein, 326 aa, Bos taurus
ASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVGVSSDCSKPNNQHCVREPSVFIFPPKPKDTLMITGTPEVTCVVVNVGHDNPEVQFSWFVDDVEVHTARTKPREEQFNSTYRVVSALPIQHQDWTGGKEFKCKVNIKGLSASIVRIISRSKGPAREPQVYVLDPPKEELSKSTVSVTCMVIGFYPEDVDVEWQRDRQTESEDKYRTTPPQLDADRSYFLYSKLRVDRNSWQRGDTYTCVVMHEALHNHYMQKSTSKSAGK

SEQ ID NO: 26 - protein, 327 aa, Bos taurus
ASTTAPKVYPLSSCCGDKSSSGVTLGCLVSSYMPEPVTVTWNSGALKSGVHTFPAVLQSSGLYSLSSMVTVPASSSGTQTFTCNVAHPASSTKVDKAVGVSSDCSKPNNQHCVREPSVFIFPPKPKDTLMITGTPEVTCVVVNVGHDNPEVQFSWFVDDVEVHTARTKPREEQFNSTYRVVSALPIQHQDWTGGKEFKCKVNIKGLSASIVRIISRSKGPAREPQVYVLDPPKEELSKSTVSLTCMVIGFYPEDVDVEWQRDRQTESEDKYRTTPPQLDADRSYFLYSKLRVDRNSWQRGDTYTCVVMHEALHNHYMQKSTSKSAGK

SEQ ID NO: 27 - protein, 352 aa, Bos taurus
ASTTAPKVYPLASSCGDTSSSTVTLGCLVSSYMPEPVTVTWNSGALKSGVHTFPAVRQSSGLYSLSSMVTVPASSSETQTFTCNVAHPASSTKVDKAVTARRPVPTTPKTTIPPGKPTTPKSEVEKTPCQCSKCPEPLGGLSVFIFPPKPKDTLTISGTPEVTCVVVDVGQDDPEVQFSWFVDDVEVHTARTKPREEQFNSTYRVVSALRIQHQDWLQGKEFKCKVNNKGLPAPIVRTISRTKGQAREPQVYVLAPPREELSKSTLSLTCLITGFYPEEIDVEWQRNGQPESEDKYHTTAPQLDADGSYFLYSKLRVNKSSWQEGDHYTCAVMHEALRNHYKEKSISRSPGK

SEQ ID NO: 28 - protein, 352 aa, Bos taurus
ASTTAPKVYPLASRCGDTSSSTVTLGCLVSSYMPEPVTVTWNSGALKSGVHTFPAVLQSSGLYSLSSMVTVPASTSETQTFTCNVAHPASSTKVDKAVTARRPVPTTPKTTIPPGKPTTQESEVEKTPCQCSKCPEPLGGLSVFIFPPKPKDTLTISGTPEVTCVVVDVGQDDPEVQFSWFVDDVEVHTARTKPREEQFNSTYRVVSALRIQHQDWLQGKEFKCKVNNKGLPAPIVRTISRTKGQAREPQVYVLAPPREELSKSTLSLTCLITGFYPEEIDVEWQRNGQPESEDKYHTTAPQLDADGSYFLYSRLRVNKSSWQEGDHYTCAVMHEALRNHYKEKSISRSPGK

SEQ ID NO: 29 - DNA, 990 bp, Bos taurus
gcctccaccacagccccgaaagtctaccctctgagttcttgctgcggggacaagtccagctccaccgtgaccctgggctgcctggtctccagctacatgcccgagccggtgaccgtgacctggaactcgggtgccctgaagagcggcgtgcacaccttcccggctgtccttcagtcctccgggctgtactctctcagcagcatggtgaccgtgcccggcagcacctcaggacagaccttcacctgcaacgtagcccacccggccagcagcaccaaggtggacaaggctgttgatcccacatgcaaaccatcaccctgtgactgttgcccaccccctgagctccccggaggaccctctgtcttcatcttcccaccgaaacccaaggacaccctcacaatctcgggaacgcccgaggtcacgtgtgtggtggtggacgtgggccacgatgaccccgaggtgaagttctcctggttcgtggacgacgtggaggtaaacacagccacgacgaagccgagagaggagcagttcaacagcacctaccgcgtggtcagcgccctgcgcatccagcaccaggactggactggaggaaaggagttcaagtgcaaggtccacaacgaaggcctcccggcccccatcgtgaggaccatctccaggaccaaagggccggcccgggagccgcaggtgtatgtcctggccccaccccaggaagagctcagcaaaagcacggtcagcctcacctgcatggtcaccagcttctacccagactacatcgccgtggagtggcagagaaacgggcagcctgagtcggaggacaagtacggcacgaccccgccccagctggacgccgacagctcctacttcctgtacagcaagctcagggtggacaggaacagctggcaggaaggagacacctacacgtgtgtggtgatgcacgaggccctgcacaatcactacacgcagaagtccacctctaagtctgcgggtaaatga

SEQ ID NO: 30 - DNA, 990 bp, Bos taurus
gcctccaccacagccccgaaagtctaccctctgagttcttgctgcggggacaagtccagctccaccgtgaccctgggctgcctggtctccagctacatgcccgagccggtgaccgtgacctggaactcgggtgccctgaagagcggcgtgcacaccttcccggccgtccttcagtcctccgggctgtactctctcagcagcatggtgaccgtgcccggcagcacctcaggacagaccttcacctgcaacgtagcccacccggccagcagcaccaaggtggacaaggctgttgatcccacatgcaaaccatcaccctgtgactgttgcccaccccctgagctccccggaggaccctctgtcttcatcttcccaccgaaacccaaggacaccctcacaatctcgggaacgcccgaggtcacgtgtgtggtggtggacgtgggccacgatgaccccgaggtgaagttctcctggttcgtggacgacgtggaggtaaacacagccacgacgaagccgagagaggagcagttcaacagcacctaccgcgtggtcagcgccctgcgcatccagcaccaggactggactggaggaaaggagttcaagtgcaaggtccacaacgaaggcctcccggcccccatcgtgaggaccatctccaggaccaaagggccggcccgggagccgcaggtgtatgtcctggccccaccccaggaagagctcagcaaaagcacggtcagcctcacctgcatggtcaccagcttctacccagactacatcgccgtggagtggcagagaaacgggcagcctgagtcggaggacaagtacggcacgaccccgccccagctggacgccgacagctcctacttcctgtacagcaagctcagggtggacaggaacagctggcaggaaggagacacctacacgtgtgtggtgatgcacgaggccctgcacaatcactacacgcagaagtccacctctaagtctgcgggtaaatga

SEQ ID NO: 31 - DNA, 990 bp, Bos taurus
gcctccaccacagccccgaaagtctaccctctgagttcttgctgcggggacaagtccagctccaccgtgaccctgggctgcctggtctccagctacatgcccgagccggtgaccgtgacctggaactcgggtgccctgaagagcggcgtgcacaccttcccggccgtccttcagtcctccgggctctactctctcagcagcatggtgaccgtgcccggcagcacctcaggaacccagaccttcacctgcaacgtagcccacccggccagcagcaccaaggtggacaaggctgttgatcccagatgcaaaacaacctgtgactgttgcccaccgcctgagctccctggaggaccctctgtcttcatcttcccaccgaaacccaaggacaccctcacaatctcgggaacgcccgaggtcacgtgtgtggtggtggacgtgggccacgatgaccccgaggtgaagttctcctggttcgtggacgacgtggaggtaaacacagccacgacgaagccgagagaggagcagttcaacagcacctaccgcgtggtcagcgccctgcgcatccagcaccaggactggactggaggaaaggagttcaagtgcaaggtccacaacgaaggcctcccagcccccatcgtgaggaccatctccaggaccaaagggccggcccgggagccgcaggtgtatgtcctggccccaccccaggaagagctcagcaaaagcacggtcagcctcacctgcatggtcaccagcttctacccagactacatcgccgtggagtggcagagaaatgggcagcctgagtcagaggacaagtacggcacgacccctccccagctggacgccgacggctcctacttcctgtacagcaggctcagggtggacaggaacagctggcaggaaggagacacctacacgtgtgtggtgatgcacgaggccctgcacaatcactacacgcagaagtccacctctaagtctgcgggtaaatga

SEQ ID NO: 32 - DNA, 981 bp, Bos taurus
gcctccaccacagccccgaaagtctaccctctggcatccagctgcggagacacatccagctccaccgtgaccctgggctgcctggtgtccagctacatgcccgagccggtgaccgtgacctggaactcgggtgccctgaagagcggcgtgcacaccttcccggctgtccttcagtcctccgggctctactctctcagcagcatggtgaccgtgcccgccagcagctcaggacagaccttcacctgcaacgtagcccacccggccagcagcaccaaggtggacaaggctgttggggtctccattgactgctccaagtgtcataaccagccttgcgtgagggaaccatctgtcttcatcttcccaccgaaacccaaagacaccctgatgatcacaggaacgcccgaggtcacgtgtgtggtggtgaacgtgggccacgataaccccgaggtgcagttctcctggttcgtggatgacgtggaggtgcacacggccaggtcgaagccaagagaggagcagttcaacagcacgtaccgcgtggtcagcgccctgcccatccagcaccaggactggactggaggaaaggagttcaagtgcaaggtcaacaacaaaggcctctcggcccccatcgtgaggatcatctccaggagcaaagggccggcccgggagccgcaggtgtatgtcctggacccacccaaggaagagctcagcaaaagcacgctcagcgtcacctgcatggtcaccggcttctacccagaagatgtagccgtggagtggcagagaaaccggcagactgagtcggaggacaagtaccgcacgaccccgccccagctggacaccgaccgctcctacttcctgtacagcaagctcagggtggacaggaacagctggcaggaaggagacgcctacacgtgtgtggtgatgcacgaggccctgcacaatcactacatgcagaagtccacctctaagtctgcgggtaaatga

SEQ ID NO: 33 - DNA, 981 bp, Bos taurus
gcctccaccacagccccgaaagtctaccctctgagttcttgctgcggggacaagtccagctccaccgtgaccctgggctgcctggtgtccagctacatgcccgagccggtgaccgtgacctggaactcgggtgccctgaagagcggcgtgcacaccttcccggccgtccttcagtcctccgggctctactctctcagcagcatggtgaccgtgcccggcagcacctcaggacagaccttcacctgcaacgtagcccacccggccagcagcaccaaggtggacaaggctgttggggtctccagtgactgctccaagcctaataaccagcattgcgtgagggaaccatctgtcttcatcttcccaccgaaacccaaagacaccctgatgatcacaggaacgcccgaggtcacgtgtgtggtggtgaacgtgggccacgataaccccgaggtgcagttctcctggttcgtggacgacgtggaggtgcacacggccaggacgaagccgagagaggagcagttcaacagcacgtaccgcgtggtcagcgccctgcccatccagcaccaggactggactggaggaaaggagttcaagtgcaaggtcaacatcaaaggcctctcggcctccatcgtgaggatcatctccaggagcaaagggccggcccgggagccgcaggtgtatgtcctggacccacccaaggaagagctcagcaaaagcacggtcagcgtcacctgcatggtcatcggcttctacccagaagatgtagacgtggagtggcagagagaccggcagactgagtcggaggacaagtaccgcacgaccccgccccagctggacgccgaccgctcctacttcctgtacagcaagctcagggtggacaggaacagctggcagagaggagacacctacacgtgtgtggtgatgcacgaggccctgcacaatcactacatgcagaagtccacctctaagtctgcgggtaaatga

SEQ ID NO: 34 - DNA, 984 bp, Bos taurus
gcctccaccacagccccgaaagtctaccctctgagttcttgctgcggggacaagtccagctcgggggtgaccctgggctgcctggtctccagctacatgcccgagccggtgaccgtgacctggaactcgggtgccctgaagagcggcgtgcacaccttcccggccgtccttcagtcctccgggctctactctctcagcagcatggtgaccgtgcccgccagcagctcaggaacccagaccttcacctgcaacgtagcccacccggccagcagcaccaaggtggacaaggctgttggggtctccagtgactgctccaagcctaataaccagcattgcgtgagggaaccatctgtcttcatcttcccaccgaaacccaaagacaccctgatgatcacaggaacgcccgaggtcacgtgtgtggtggtgaacgtgggccacgataaccccgaggtgcagttctcctggttcgtggacgacgtggaggtgcacacggccaggacgaagccgagagaggagcagttcaacagcacgtaccgcgtggtcagcgccctgcccatccagcaccaggactggactggaggaaaggagttcaagtgcaaggtcaacatcaaaggcctctcggcctccatcgtgaggatcatctccaggagcaaagggccggcccgggagccgcaggtgtatgtcctggacccacccaaggaagagctcagcaaaagcacggtcagcctcacctgcatggtcatcggcttctacccagaagatgtagacgtggagtggcagagagaccggcagactgagtcggaggacaagtaccgcacgaccccgccccagctggacgccgaccgctcctacttcctgtacagcaagctcagggtggacaggaacagctggcagagaggagacacctacacgtgtgtggtgatgcacgaggccctgcacaatcactacatgcagaagtccacctctaagtctgcgggtaaatga

SEQ ID NO: 35 - DNA, 1059 bp, Bos taurus
gcctccaccacagccccgaaagtctaccctctggcatccagctgcggagacacatccagctccaccgtgaccctgggctgcctggtctccagctacatgcccgagccggtgaccgtgacctggaactcgggtgccctgaagagcggcgtgcacaccttcccggccgtccggcagtcctctgggctgtactctctcagcagcatggtgactgtgcccgccagcagctcagaaacccagaccttcacctgcaacgtagcccacccggccagcagcaccaaggtggacaaggctgtcactgcaaggcgtccagtcccgacgacgccaaagacaactatccctcctggaaaacccacaaccccaaagtctgaagttgaaaagacaccctgccagtgttccaaatgcccagaacctctgggaggactgtctgtcttcatcttcccaccgaaacccaaggacaccctcacaatctcgggaacgcccgaggtcacgtgtgtggtggtggacgtgggccaggatgaccccgaggtgcagttctcctggttcgtggacgacgtggaggtgcacacggccaggacgaagccgagagaggagcagttcaacagcacctaccgcgtggtcagcgccctgcgcatccagcaccaggactggctgcagggaaaggagttcaagtgcaaggtcaacaacaaaggcctcccggcccccattgtgaggaccatctccaggaccaaagggcaggcccgggagccgcaggtgtatgtcctggccccaccccgggaagagctcagcaaaagcacgctcagcctcacctgcctgatcaccggtttctacccagaagagatagacgtggagtggcagagaaatgggcagcctgagtcggaggacaagtaccacacgaccgcaccccagctggatgctgacggctcctacttcctgtacagcaagctcagggtgaacaagagcagctggcaggaaggagaccactacacgtgtgcagtgatgcacgaagctttacggaatcactacaaagagaagtccatctcgaggtctccgggtaaatga

SEQ ID NO: 36 - DNA, 1059 bp, Bos taurus
gcctccaccacagccccgaaagtctaccctctggcatcccgctgcggagacacatccagctccaccgtgaccctgggctgcctggtctccagctacatgcccgagccggtgaccgtgacctggaactcgggtgccctgaagagtggcgtgcacaccttcccggccgtccttcagtcctccgggctgtactctctcagcagcatggtgaccgtgcccgccagcacctcagaaacccagaccttcacctgcaacgtagcccacccggccagcagcaccaaggtggacaaggctgtcactgcaaggcgtccagtcccgacgacgccaaagacaaccatccctcctggaaaacccacaacccaggagtctgaagttgaaaagacaccctgccagtgttccaaatgcccagaacctctgggaggactgtctgtcttcatcttcccaccgaaacccaaggacaccctcacaatctcgggaacgcccgaggtcacgtgtgtggtggtggacgtgggccaggatgaccccgaggtgcagttctcctggttcgtggacgacgtggaggtgcacacggccaggacgaagccgagagaggagcagttcaacagcacctaccgcgtggtcagcgccctgcgcatccagcaccaggactggctgcagggaaaggagttcaagtgcaaggtcaacaacaaaggcctcccggcccccattgtgaggaccatctccaggaccaaagggcaggcccgggagccgcaggtgtatgtcctggccccaccccgggaagagctcagcaaaagcacgctcagcctcacctgcctgatcaccggtttctacccagaagagatagacgtggagtggcagagaaatgggcagcctgagtcggaggacaagtaccacacgaccgcaccccagctggatgctgacggctcctacttcctgtacagcaggctcagggtgaacaagagcagctggcaggaaggagaccactacacgtgtgcagtgatgcatgaagctttacggaatcactacaaagagaagtccatctcgaggtctccgggtaaatga

SEQ ID NO: 37 - protein, 331 aa, Ovis aries
ASTTPPKVYPLTSCCGDTSSSIVTLGCLVSSYMPEPVTVTWNSGALTSGVHTFPAILQSSGLYSLSSVVTVPASTSGAQTFICNVAHPASSTKVDKRVEPGCPDPCKHCRCPPPELPGGPSVFIFPPKPKDTLTISGTPEVTCVVVDVGQDDPEVQFSWFVDNVEVRTARTKPREEQFNSTFRVVSALPIQHQDWTGGKEFKCKVHNEALPAPIVRTISRTKGQAREPQVYVLAPPQEELSKSTLSVTCLVTGFYPDYIAVEWQKNGQPESEDKYGTTTSQLDADGSYFLYSRLRVDKNSWQEGDTYACVVMHEALHNHYTQKSISKPPGK

SEQ ID NO: 38 - DNA, 996 bp, Ovis aries
gcctcaacaacacccccgaaagtctaccctctgacttcttgctgcggggacacgtccagctccatcgtgaccctgggctgcctggtctccagctatatgcccgagccggtgaccgtgacctggaactctggtgccctgaccagcggcgtgcacaccttcccggccatcctgcagtcctccgggctctactctctcagcagcgtggtgaccgtgccggccagcacctcaggagcccagaccttcatctgcaacgtagcccacccggccagcagcaccaaggtggacaagcgtgttgagcccggatgcccggacccatgcaaacattgccgatgcccaccccctgagctccccggaggaccgtctgtcttcatcttcccaccgaaacccaaggacacccttacaatctctggaacgcccgaggtcacgtgtgtggtggtggacgtgggccaggatgaccccgaggtgcagttctcctggttcgtggacaacgtggaggtgcgcacggccaggacaaagccgagagaggagcagttcaacagcaccttccgcgtggtcagcgccctgcccatccagcaccaagactggactggaggaaaggagttcaagtgcaaggtccacaacgaagccctcccggcccccatcgtgaggaccatctccaggaccaaagggcaggcccgggagccgcaggtgtacgtcctggccccaccccaggaagagctcagcaaaagcacgctcagcgtcacctgcctggtcaccggcttctacccagactacatcgccgtggagtggcagaaaaatgggcagcctgagtcggaggacaagtacggcacgaccacatcccagctggacgccgacggctcctacttcctgtacagcaggctcagggtggacaagaacagctggcaagaaggagacacctacgcgtgtgtggtgatgcacgaggctctgcacaaccactacacacagaagtcgatctctaagcctccgggtaaatga

SEQ ID NO: 39 - protein, 329 aa, Ovis aries
ASTTAPKVYPLTSCCGDTSSSSSIVTLGCLVSSYMPEPVTVTWNSGALTSGVHTFPAILQSSGLYSLSSVVTVPASTSGAQTFICNVAHPASSAKVDKRVGISSDYSKCSKPPCVSRPSVFIFPPKPKDSLMITGTPEVTCVVVDVGQGDPEVQFSWFVDNVEVRTARTKPREEQFNSTFRVVSALPIQHDHWTGGKEFKCKVHSKGLPAPIVRTISRAKGQAREPQVYVLAPPQEELSKSTLSVTCLVTGFYPDYIAVEWQRARQPESEDKYGTTTSQLDADGSYFLYSRLRVDKSSWQRGDTYACVVMHEALHNHYTQKSISKPPGK

SEQ ID NO: 40 - DNA, 990 bp, Ovis aries
gcctccaccacagccccgaaagtctaccctctgacttcttgctgcggggacacgtccagctccagctccatcgtgaccctgggctgcctggtctccagctatatgcccgagccggtgaccgtgacctggaactctggtgccctgaccagcggcgtgcacaccttcccggccatcctgcagtcctccgggctctactctctcagcagcgtggtgaccgtgccggccagcacctcaggagcccagaccttcatctgcaacgtagcccacccggccagcagcgccaaggtggacaagcgtgttgggatctccagtgactactccaagtgttctaaaccgccttgcgtgagccgaccgtctgtcttcatcttccccccgaaacccaaggacagcctcatgatcacaggaacgcccgaggtcacgtgtgtggtggtggacgtgggccagggtgaccccgaggtgcagttctcctggttcgtggacaacgtggaggtgcgcacggccaggacaaagccgagagaggagcagttcaacagcaccttccgcgtggtcagcgccctgcccatccagcacgaccactggactggaggaaaggagttcaagtgcaaggtccacagcaaaggcctcccggcccccatcgtgaggaccatctccagggccaaagggcaggcccgggagccgcaggtgtacgtcctggccccaccccaggaagagctcagcaaaagcacgctcagcgtcacctgcctggtcaccggcttctacccagactacatcgccgtggagtggcagagagcgcggcagcctgagtcggaggacaagtacggcacgaccacatcccagctggacgccgacggctcctacttcctgtacagcaggctcagggtggacaagagcagctggcaaagaggagacacctacgcgtgtgtggtgatgcacgaggctctgcacaaccactacacacagaagtcgatctctaagcctccgggtaaatga

SEQ ID NO: 41 - protein, 102 aa, Ovis aries
PSVFLFKPSEEQLRTGTVSVVCLVNDFYPKDINVKVKVDGVTQNSNFQNSFTDQDSKKSTYSLSSTLTLSSSEYQSHNAYACEVSHKSLPTALVKSFNKNEC

SEQ ID NO: 42 - DNA, 309 bp, Ovis aries
ccatccgtcttcctcttcaaaccatctgaggaacagctgaggaccggaactgtctctgtcgtgtgcttggtgaatgatttctaccccaaagatatcaatgtcaaggtgaaagtggatggggttacccagaacagcaacttccagaacagcttcacagaccaggacagcaagaaaagcacctacagcctcagcagcaccctgacactgtccagctcagagtaccagagccataacgcctatgcgtgtgaggtcagccacaagagcctgcccaccgccctcgtcaagagcttcaataagaatgaatgttag

SEQ ID NO: 43 - protein, 106 aa, Ovis aries
GQPKSAPSVTLFPPSTEELSTNKATVVCLINDFYPGSVNVVWKADGSTINQNVKTTQASKQSNSKYAASSYLTLTGSEWKSKSSYTCEVTHEGSTVTKTVKPSECS

SEQ ID NO: 44 - DNA, 321 bp, Ovis aries
ggtcagcccaagtccgcaccctcggtcaccctgttcccgccttccacggaggagctcagtaccaacaaggccaccgtggtgtgtctcatcaacgacttctacccgggtagcgtgaacgtggtctggaaggcagatggcagcaccatcaatcagaacgtgaagaccacccaggcctccaaacagagcaacagcaagtacgcggccagcagctacctgaccctgacgggcagcgagtggaagtctaagagcagttacacctgcgaggtcacgcacgaggggagcaccgtgacgaagacagtgaagccctcagagtgttcttag

SEQ ID NO: 45 - protein, 266 aa, Bubalus bubalis
SGVHTFPAVLQSSGLYSLSSTVTAPASATKSQTFTCNVAHPASSTKVDKAVVPPCRPKPCDCCPPPELPGGPSVFIFPPKPKDTLTISGTPEVTCVVVDVGHDDPEVKFSWFVDDVEVNTARTKPREEQFNSTYRVVSALPIQHNDWTGGKEFKCKVYNEGLPAPIVRTISRTKGQAREPQVYVLAPPQDELSKSTVSITCMVTGFYPDYIAVEWQKDGQPESEDKYGTTPPQLDSDGSYFLYSRLRVNKNSWQEGGAYTCVVMHE

SEQ ID NO: 46 - DNA, 801 bp, Bubalus bubalis
gagcggcgtgcacaccttcccggccgtccttcagtcctccgggctctactctctcagcagcacggtgaccgcgcccgccagcgccacaaaaagccagaccttcacctgcaacgtagcccacccggccagcagcaccaaggtggacaaggctgttgttcccccatgcagaccgaaaccctgtgattgctgcccaccccctgagctccccggaggaccctctgtcttcatcttcccaccaaaacccaaggacaccctcacaatctctggaactcctgaggtcacgtgtgtggtggtggacgtgggccacgatgaccccgaggtgaagttctcctggttcgtggacgatgtggaggtaaacacagccaggacgaagccaagagaggagcagttcaacagcacctaccgcgtggtcagcgccctgcccatccagcacaacgactggactggaggaaaggagttcaagtgcaaggtctacaatgaaggcctcccagcccccatcgtgaggaccatctccaggaccaaagggcaggcccgggagccgcaggtgtacgtcctggccccaccccaggacgagctcagcaaaagcacggtcagcatcacttgcatggtcactggcttctacccagactacatcgccgtagagtggcagaaagatgggcagcctgagtcagaggacaaatatggcacgaccccgccccagctggacagcgatggctcctacttcctgtacagcaggctcagggtgaacaagaacagctggcaagaaggaggcgcctacacgtgtgtagtgatgcatgaggc

SEQ ID NO: 47 - protein, 309 aa, Bubalus bubalis
ASITAPKVYPLTSCRGETSSSTVTLGCLVSSYMPEPVTVTWNSGALKSGVHTFPAVLQSSGLYSLSSTVTAPASATKSQTFTCNVAHPASSTKVDTAVGFSSDCCKFPKPCVRGPSVFIFPPKPKDTLMITGNPEVTCVVVDVGRDNPEVQFSWFVGDVEVHTGRSKPREEQFNSTYRVVSTLPIQHNDWTGGKEFKCKVNNKGLPAPIVRTISRTKGQAREPQVYVLAPPQEELSKSTVSVTCMVTGFYPDYIAVEWHRDRQAESEDKYRTTPPQLDSDGSYFLYSRLKVNKNSWQEGGAYTCVVMHE

SEQ ID NO: 48 - DNA, 929 bp, Bubalus bubalis
gcctccatcacagccccgaaagtctaccctctgacttcttgccgcggggaaacgtccagctccaccgtgaccctgggctgcctggtctccagctacatgcccgagccggtgaccgtgacctggaactcgggtgccctgaagagcggcgtgcacaccttcccggccgtccttcagtcctctgggctctactctctcagcagcacggtgaccgcgcccgccagcgccacaaaaagccagaccttcacctgcaacgtagcccacccggccagcagcaccaaggtggacacggctgttgggttctccagtgactgctgcaagtttcctaagccttgtgtgaggggaccatctgtcttcatcttcccgccgaaacccaaagacaccctgatgatcacaggaaatcccgaggtcacatgtgtggtggtggacgtgggccgggataaccccgaggtgcagttctcctggttcgtgggtgatgtggaggtgcacacgggcaggtcgaagccgagagaggagcagttcaacagcacctaccgcgtggtcagcaccctgcccatccagcacaatgactggactggaggaaaggagttcaagtgcaaggtcaacaacaaaggcctcccagcccccatcgtgaggaccatctccaggaccaaagggcaggcccgggagccgcaggtgtacgtcctggccccaccccaggaagagctcagcaaaagcacggtcagcgtcacttgcatggtcactggcttctacccagactacatcgccgtagagtggcatagagaccggcaggctgagtcggaggacaagtaccgcacgaccccgccccagctggacagcgatggctcctacttcctgtacagcaggctcaaggtgaacaagaacagctggcaagaaggaggcgcctacacgtgtgtagtgatgcatgaggc

SEQ ID NO: 49 - protein, 352 aa, Bubalus bubalis
ASTTAPKVYPLASSCGDTSSSTVTLGCLVSSYMPEPVTVTWNSGALKNGVHTFPAVRQSSGLYSLSSMVTMPTSTAGTQTFTCNVAHPASSTKVDTAVTARHPVPKTPETPIHPVKPPTQEPRDEKTPCQCPKCPEPLGGLSVFIFPPKPKDTLTISGTPEVTCVVVDVGQDDPEVQFSWFVDDVEVHTARMKPREEQFNSTYRVVSALPIQHQDWLREKEFKCKVNNKGLPAPIVRTISRTKGQAREPQVYVLAPPREELSKSTLSLTCLITGFYPEEVDVEWQRNGQPESEDKYHTTPPQLDADGSYFLYSRLRVNRSSWQEGDHYTCAVMHEALRNHYKEKPISRSPGK

SEQ ID NO: 50 - DNA, 1059 bp, Bubalus bubalis
gcctccaccacagccccgaaagtctaccctctggcatccagctgcggggacacgtccagctccaccgtgaccctgggctgcctggtctccagctacatgcccgagccggtgaccgtgacctggaactcgggtgccctgaagaacggcgtgcacaccttcccggccgtccggcagtcctccgggctctactctctcagcagcatggtgaccatgcccaccagcaccgcaggaacccagaccttcacctgcaacgtagcccacccggccagcagcaccaaggtggacacggctgtcactgcaaggcatccggtcccgaagacaccagagacacctatccatcctgtaaaacccccaacccaggagcccagagatgaaaagacaccctgccagtgtcccaaatgcccagaacctctgggaggactgtctgtcttcatcttcccaccgaaacccaaggacaccctcacaatctctggaacgcccgaggtcacgtgtgtggtggtggacgtgggccaggatgaccccgaagtgcagttctcctggttcgtggatgacgtggaggtgcacacagccaggatgaagccaagagaggagcagttcaacagcacctaccgcgtggtcagcgccctgcccatccagcaccaggactggctgcgggaaaaggagttcaagtgcaaggtcaacaacaaaggcctcccggcccccatcgtgaggaccatctccaggaccaaagggcaggcccgggagccacaggtgtatgtcctggccccaccccgggaagagctcagcaaaagcacgctcagcctcacctgcctaatcaccggcttctacccagaagaggtagacgtggagtggcagagaaatgggcagcctgagtcagaggacaagtaccacacgaccccaccccagctggacgctgacggctcctacttcctgtacagcaggctcagggtgaacaggagcagctggcaggaaggagaccactacacgtgtgcagtgatgcatgaagctttacggaatcactacaaagagaagcccatctcgaggtctccgggtaaatga

SEQ ID NO: 51 - protein, 105 aa, Bubalus bubalis
QPKSAPSVTLFPPSTEELSANKATLVCLISDFYPGSMTVARKADGSTITRNVETTRASKQSNSKYAASSYLSLTGSEWKSKGSYSCEVTHEGSTVTKTVKPSECS

SEQ ID NO: 52 - DNA, 318 bp, Bubalus bubalis
cagcccaagtccgcaccctcagtcaccctgttcccaccctccacggaggagctcagcgccaacaaggccaccctggtgtgtctcatcagcgacttctacccgggtagcatgaccgtggccaggaaggcagacggcagcaccatcacccggaacgtggagaccacccgggcctccaaacagagcaacagcaagtacgcggccagcagctacctgagcctgacgggcagcgagtggaaatcgaaaggcagttacagctgcgaggtcacgcacgaggggagcaccgtgacaaagacagtgaagccctcagagtgttcttag

SEQ ID NO: 53 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - DNA, 690 bp, Homo sapiens
gagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 55 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 56 - DNA, 690 bp, Homo sapiens
gagtccaaatatggtcccccgtgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcgtgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacgcagaagagcctctccctgtctctgggtaaatga

SEQ ID NO: 57 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 58 - DNA, 654 bp, Homo sapiens
gcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctcaccgtggacaagagcaggtggcaggaggggaacgtcttctcatgctccgtgatgcatgaggctctgcacaaccactacacgcagaagagcctctccctgtctctgggtaaatga

SEQ ID NO: 59 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - DNA, 321 bp, Homo sapiens
actgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 61 - DNA, 32 bp, Artificial Sequence
  primer
atatgcggccgcatggggaccccgcgggcgct

SEQ ID NO: 62 - DNA, 30 bp, Artificial Sequence
  primer
gcgcaagctttcagaggggccaggagcagt

SEQ ID NO: 63 - DNA, 35 bp, Artificial Sequence
  primer
ctagctagcaccatgaggatatatagtgtcttaac

SEQ ID NO: 64 - DNA, 31 bp, Artificial Sequence
  primer
caatctcgagttacagacagaagatgactgc

SEQ ID NO: 65 - DNA, 40 bp, Artificial Sequence
  primer
ataagaatgcggccgccaccatggggaccccgcgggcgct

SEQ ID NO: 66 - DNA, 50 bp, Artificial Sequence
  primer
gccctcgagttaatggtgatggtgatggtggatgaccaggctctgcatct

SEQ ID NO: 67 - DNA, 20 bp, Artificial Sequence
  primer
atggggaccccgcgggcgcc

SEQ ID NO: 68 - DNA, 25 bp, Artificial Sequence
  primer
tcagaggggccaggagcagtgtcca

SEQ ID NO: 69 - DNA, 20 bp, Artificial Sequence
  primer
atggggaccccgcgggcgct

SEQ ID NO: 70 - DNA, 20 bp, Artificial Sequence
  primer
gatgaccaggctctgcatct

SEQ ID NO: 71 - DNA, 20 bp, Artificial Sequence
  primer
aatgacagcggcgtctactt

SEQ ID NO: 72 - DNA, 20 bp, Artificial Sequence
  primer
tcagaggggccaggagcagt

SEQ ID NO: 73 - DNA, 29 bp, Artificial Sequence
  primer
gaagatctatggggaccccgcgggcgccg

SEQ ID NO: 74 - DNA, 29 bp, Artificial Sequence
  primer
gacccggggaggggccaggagcagtgtcc

SEQ ID NO: 75 - DNA, 1404 bp, Artificial Sequence
  DNA encoding chimeric H chain
atggcaatcctcgtgttgcttctgtgcttggtgaccattccacactctgtgctttcccaggtgcagctcaaggaaacagggccaggactcgtccaacctacacaaaccctgtcaatcacctgtaccgtatccggttttagcctcaccagctattatatacaatgggtgaggcagacccccgggaaaggactggaatggatgggcttcattcgcagcggtgggagtaccgagtacaatagcgagtttaaaagtcgcttgagtatcaatagagatacttccaagaatcaggtgttcttgaagatgaactccctcaagaccgaagatacaggggtctattactgcgccaggacctccagtggatatgaaggaggctttgattattgggggcagggcgtcatggtaactgtgagctcagcctccaccacagccccgaaagtctaccctctgagttcttgctgcggggacaagtccagctccaccgtgaccctgggctgcctggtctccagctacatgcccgagccggtgaccgtgacctggaactcgggtgccctgaagagcggcgtgcacaccttcccggctgtccttcagtcctccgggctgtactctctcagcagcatggtgaccgtgcccggcagcacctcaggacagaccttcacctgcaacgtagcccacccggccagcagcaccaaggtggacaaggctgttgatcccacatgcaaaccatcaccctgtgactgttgcccaccccctgagctccccggaggaccctctgtcttcatcttcccaccgaaacccaaggacaccctcacaatctcgggaacgcccgaggtcacgtgtgtggtggtggacgtgggccacgatgaccccgaggtgaagttctcctggttcgtggacgacgtggaggtaaacacagccacgacgaagccgagagaggagcagttcaacagcacctaccgcgtggtcagcgccctgcgcatccagcaccaggactggactggaggaaaggagttcaagtgcaaggtccacaacgaaggcctcccggcccccatcgtgaggaccatctccaggaccaaagggccggcccgggagccgcaggtgtatgtcctggccccaccccaggaagagctcagcaaaagcacggtcagcctcacctgcatggtcaccagcttctacccagactacatcgccgtggagtggcagagaaacgggcagcctgagtcggaggacaagtacggcacgaccccgccccagctggacgccgacagctcctacttcctgtacagcaagctcagggtggacaggaacagctggcaggaaggagacacctacacgtgtgtggtgatgcacgaggccctgcacaatcactacacgcagaagtccacctctaagtctgcgggtaaataa

SEQ ID NO: 76 - protein, 467 aa, Artificial Sequence
  chimeric H chain
MAILVLLLCLVTIPHSVLSQVQLKETGPGLVQPTQTLSITCTVSGFSLTSYYIQWVRQTPGKGLEWMGFIRSGGSTEYNSEFKSRLSINRDTSKNQVFLKMNSLKTEDTGVYYCARTSSGYEGGFDYWGQGVMVTVSSASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVDPTCKPSPCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 77 - DNA, 28 bp, Artificial Sequence
  primer
ctagctagcaccacagccccgaaagtct

SEQ ID NO: 78 - DNA, 30 bp, Artificial Sequence
  primer
tgctctagattatttacccgcagacttaga

SEQ ID NO: 79 - DNA, 40 bp, Artificial Sequence
  primer
ataagaatgcggccgccaccatgtggctcataatagctct

SEQ ID NO: 80 - DNA, 50 bp, Artificial Sequence
  primer
gccctcgagttaatggtgatggtgatggtgaggagttgttgactggaggc

SEQ ID NO: 81 - DNA, 38 bp, Artificial Sequence
  primer
ataagaatgctagccaccatggggatcccctcattcct

SEQ ID NO: 82 - DNA, 50 bp, Artificial Sequence
  primer
gccgatatcttaatggtgatggtgatggtgcgatgaggggccgctcgagc

SEQ ID NO: 83 - DNA, 40 bp, Artificial Sequence
  primer
ataagaatgcggccgccaccatgtggcaactgctaccacc

SEQ ID NO: 84 - DNA, 50 bp, Artificial Sequence
  primer
gccctcgagttaatggtgatggtgatggtggtgccaaggtagaaagaatg

SEQ ID NO: 85 - DNA, 48 bp, Artificial Sequence
  primer
ataagaatgcggccgccaccatggcccccaccctccctgccttgctct

SEQ ID NO: 86 - DNA, 50 bp, Artificial Sequence
  primer
gccctcgagttaatggtgatggtgatggtgattctgcatcgtgtagtctg